SEQUENCE LISTING (64 sequences)

SEQ ID NO: 1 - protein, 15 aa, Artificial Sequence
  Description of Artificial Sequence Synthetic peptide
GGGGSGGGGSGGGGS

SEQ ID NO: 2 - DNA, 45 bp, Artificial Sequence
  Description of Artificial Sequence Synthetic oligonucleotide
ggcggcggcggatctggaggtggtggctcaggtggcggaggctcc

SEQ ID NO: 3 - protein, 21 aa, Homo sapiens
MALPVTALLLPLALLLHAARP

SEQ ID NO: 4 - DNA, 63 bp, Homo sapiens
atggccctgccagtaacggctctgctgctgccacttgctctgctcctccatgcagccaggcct

SEQ ID NO: 5 - protein, 18 aa, Homo sapiens
MALPVTALLLPLALLLHA

SEQ ID NO: 6 - DNA, 54 bp, Homo sapiens
atggctctcccagtgactgccctactgcttcccctagcgcttctcctgcatgca

SEQ ID NO: 7 - protein, 21 aa, Mus musculus
MASPLTRFLSLNLLLLGESII

SEQ ID NO: 8 - DNA, 63 bp, Mus musculus
atggccagccccctgaccaggttcctgagcctgaacctgctgctgctgggcgagagcatcatc

SEQ ID NO: 9 - protein, 18 aa, Mus musculus
MASPLTRFLSLNLLLLGE

SEQ ID NO: 10 - DNA, 54 bp, Mus musculus
atggccagccccctgaccaggttcctgagcctgaacctgctgctgctgggcgag

SEQ ID NO: 11 - protein, 214 aa, Homo sapiens
MLRLLLALNLFPSIQVTGNKILVKQSPMLVAYDNALSCKYSYNLFSREFRASLHKGLDSAVEVCWYGNYSQQLQVYSKTGFNCDGKLGNESVTFYLQNLYQTDIYFCKIEVMYPPPYLDNEKSNGTIIHVKGKHLCPSPLFPGPSKPFWVLVWGGVLACYSLLVTVAFIIFWVRSKRSRLLHSDYMNMTPRRPGPTRKHYQPYAPPRDFAAYRS

SEQ ID NO: 12 - DNA, 321 bp, Homo sapiens
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 13 - protein, 234 aa, Unknown
  Description of Unknown CD8 sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPWKSGDKPSLSARYV

SEQ ID NO: 14 - protein, 71 aa, Unknown
  Description of Unknown CD8 sequence
PTTTPAPRPPTPAPTIASQPLSLRPEACRPAAGGAVHTRGLDFACDIYIWAPLAGTCGVLLLSLVITLYCN

SEQ ID NO: 15 - DNA, 213 bp, Unknown
  Description of Unknown CD8 sequence
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 16 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - protein, 164 aa, Unknown; identical to SEQ ID NO: 16
  Description of Unknown CD3-zeta sequence

SEQ ID NO: 18 - protein, 112 aa, Unknown
  Description of Unknown CD3-zeta sequence
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 112 aa, Unknown
  Description of Unknown CD3-zeta sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - DNA, 337 bp, Unknown
  Description of Unknown CD3-zeta sequence
agagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcg

SEQ ID NO: 21 - DNA, 339 bp, Unknown
  Description of Unknown CD3-zeta sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 22 - protein, 251 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLGTKERDWCGPSPADLSPGASSVTPPAPAREPGHSPQIISFFLALTSTALLFLLFFLTLRFSWKRGRKKLLYIFKQPFMRPVQTTQEEDGCSCRFPEEEEGGCEL

SEQ ID NO: 23 - protein, 42 aa, Unknown
  Description of Unknown 4-1BB sequence
KRGRKKLLYIFKQPFMRPVQTTQEEDGCSCRFPEEEEGGCEL

SEQ ID NO: 24 - DNA, 126 bp, Unknown
  Description of Unknown 4-1BB sequence
aaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 25 - protein, 276 aa, Homo sapiens
MCVGARRLGRGPCAALLLLGLGLSTVTGLHCVGDTYPSNDRCCHECRPGNGMVSRCSRSQNTVCRPCGPGFYNDWSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 26 - protein, 196 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVWCILGCILICWLTKKKYSSSVHDPNGEYMFMRATAKKSRLTDVTL

SEQ ID NO: 27 - protein, 220 aa, Homo sapiens
MACLGFQRHKAQLNLATRTWPCTLLFFLLFIPVFCKAMHVAQPAWLASSRGIASFVCEYASPGKATEVRVTVLRQADSQVTEVCAATYMMGNELTFLDDSICTGTSSGNQLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 28 - protein, 286 aa, Homo sapiens
MQIPQAPWPVVWAVLQLGWRPGWFLDSPDRPWNPPTFSPALLWTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGWGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 29 - protein, 524 aa, Homo sapiens
MWEAQFLGLLFLQPLWVAPVKPLQPGAEVPWWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 30 - protein, 368 aa, Homo sapiens
MLGQWTLILLLLLKVYQGKGCQGSADHWSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 31 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 32 - protein, 7 aa, Artificial Sequence
  Description of Artificial Sequence Synthetic peptide
GFTFSNY

SEQ ID NO: 33 - protein, 6 aa, Artificial Sequence
  Description of Artificial Sequence Synthetic peptide
STGGGN

SEQ ID NO: 34 - protein, 11 aa, Artificial Sequence
  Description of Artificial Sequence Synthetic peptide
QGGGYSDSFDY

SEQ ID NO: 35 - protein, 9 aa, Artificial Sequence
  Description of Artificial Sequence Synthetic peptide
GFSLSTSGM

SEQ ID NO: 36 - protein, 5 aa, Artificial Sequence
  Description of Artificial Sequence Synthetic peptide
WWDDD

SEQ ID NO: 37 - protein, 10 aa, Artificial Sequence
  Description of Artificial Sequence Synthetic peptide
IGGSSGYMDY

SEQ ID NO: 38 - protein, 11 aa, Artificial Sequence
  Description of Artificial Sequence Synthetic peptide
KASKSISKYLA

SEQ ID NO: 39 - protein, 7 aa, Artificial Sequence
  Description of Artificial Sequence Synthetic peptide
SGSTLQS

SEQ ID NO: 40 - protein, 9 aa, Artificial Sequence
  Description of Artificial Sequence Synthetic peptide
QQHNEYPLT

SEQ ID NO: 41 - protein, 15 aa, Artificial Sequence
  Description of Artificial Sequence Synthetic peptide
RASESVDSYGNSFMH

SEQ ID NO: 42 - protein, 7 aa, Artificial Sequence
  Description of Artificial Sequence Synthetic peptide
RASNLKS

SEQ ID NO: 43 - protein, 9 aa, Artificial Sequence
  Description of Artificial Sequence Synthetic peptide
QQSNEDPWT

SEQ ID NO: 44 - protein, 11 aa, Artificial Sequence
  Description of Artificial Sequence Synthetic peptide
KASENVVTYVS

SEQ ID NO: 45 - protein, 7 aa, Artificial Sequence
  Description of Artificial Sequence Synthetic peptide
GASNRYT

SEQ ID NO: 46 - protein, 9 aa, Artificial Sequence
  Description of Artificial Sequence Synthetic peptide
GQGYSYPYT

SEQ ID NO: 47 - protein, 120 aa, Artificial Sequence
  Description of Artificial Sequence Synthetic polypeptide
EVQLVESGGGLVQPGRSLKLSCAASGFTFSNYAMAWVRQAPTKGLEWVASISTGGGNTYYRDSVKGRFTISRDNAKNTLYLQMDSLRSEDTATYYCARQGGGYSDSFDYWGQGVMVTVSS

SEQ ID NO: 48 - protein, 120 aa, Artificial Sequence
  Description of Artificial Sequence Synthetic polypeptide
QVTLKESGPGILQPSQTLSLTCSFSGFSLSTSGMGVGWIRQPSGKGLEWLAHIWWDDDKRYNPALKSRLTISKDPSSNQVFLKIASVDTADIATYYCVRIGGSSGYMDYWGQGTSVTVSS

SEQ ID NO: 49 - protein, 108 aa, Artificial Sequence
  Description of Artificial Sequence Synthetic polypeptide
DVQMTQSPSNLAASPGESVSINCKASKSISKYLAWYQQKPGKANKLLIYSGSTLQSGTPSRFSGSGSGTDFTLTIRNLEPEDFGLYYCQQHNEYPLTFGSGTKLEIKR

SEQ ID NO: 50 - protein, 112 aa, Artificial Sequence
  Description of Artificial Sequence Synthetic polypeptide
DIVLTQSPASLAVSLGQRATISCRASESVDSYGNSFMHWYQQKPGQPPKLLIYRASNLKSGIPARFSGSGSGTDFTLTINPVEADDVATYCCQQSNEDPWTFGGGTKLEIKR

SEQ ID NO: 51 - protein, 108 aa, Artificial Sequence
  Description of Artificial Sequence Synthetic polypeptide
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKR

SEQ ID NO: 52 - protein, 243 aa, Artificial Sequence
  Description of Artificial Sequence Synthetic polypeptide
EVQLVESGGGLVQPGRSLKLSCAASGFTFSNYAMAWVRQAPTKGLEWVASISTGGGNTYYRDSVKGRFTISRDNAKNTLYLQMDSLRSEDTATYYCARQGGGYSDSFDYWGQGVMVTVSSGGGGSGGGGSGGGGSDVQMTQSPSNLAASPGESVSINCKASKSISKYLAWYQQKPGKANKLLIYSGSTLQSGTPSRFSGSGSGTDFTLTIRNLEPEDFGLYYCQQHNEYPLTFGSGTKLEIKR

SEQ ID NO: 53 - protein, 247 aa, Artificial Sequence
  Description of Artificial Sequence Synthetic polypeptide
QVTLKESGPGILQPSQTLSLTCSFSGFSLSTSGMGVGWIRQPSGKGLEWLAHIWWDDDKRYNPALKSRLTISKDPSSNQVFLKIASVDTADIATYYCVRIGGSSGYMDYWGQGTSVTVSSGGGGSGGGGSGGGGSDIVLTQSPASLAVSLGQRATISCRASESVDSYGNSFMHWYQQKPGQPPKLLIYRASNLKSGIPARFSGSGSGTDFTLTINPVEADDVATYCCQQSNEDPWTFGGGTKLEIKR

SEQ ID NO: 54 - protein, 243 aa, Artificial Sequence
  Description of Artificial Sequence Synthetic polypeptide
QVTLKESGPGILQPSQTLSLTCSFSGFSLSTSGMGVGWIRQPSGKGLEWLAHIWWDDDKRYNPALKSRLTISKDPSSNQVFLKIASVDTADIATYYCVRIGGSSGYMDYWGQGTSVTVSSGGGGSGGGGSGGGGSNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKR

SEQ ID NO: 55 - DNA, 735 bp, Artificial Sequence
  Description of Artificial Sequence Synthetic polynucleotide
gaagtccaactcgttgaaagcggcggtggtcttgtccagccaggcagatcactgaaactgtcatgcgccgccagtggcttcactttctccaattacgcaatggcgtgggttagacaggcccccacgaaaggcttggagtgggtcgcatcaatcagtacaggaggtggaaacacttactatcgcgatagtgttaaggggagattcacgattagccgggacaacgcgaaaaacacgttgtatctgcagatggactcacttagatccgaggacacagcgacttactactgtgcgaggcagggcggagggtatagtgatagctttgattactggggccagggcgtaatggtaactgttagttctggtggaggtggatcaggtggaggtggatctggtggaggtggatctgatgtgcagatgacacagagtccttcaaatttggccgcttcacccggagaatcagtaagtatcaactgtaaagcgtccaagtccatttcaaagtatttggcatggtatcaacagaagccgggaaaggcgaacaaactcctgatttatagcgggagtaccttgcagtccggcacgcctagtagattttcaggctccggttctgggaccgacttcactttgacgattcgcaatttggaaccagaggattttgggctgtactattgtcagcagcacaacgaatacccgttgacttttggtagtggtacaaagctggaaatcaagagagcggcc

SEQ ID NO: 56 - DNA, 741 bp, Artificial Sequence
  Description of Artificial Sequence Synthetic polynucleotide
caggtgaccctgaaggagtccggccccggcatcctgcagcccagccagaccctgagcctgacctgctccttcagcggcttctccctgtccacctccggcatgggcgtgggctggatcagacagcccagcggcaagggcctggagtggctggcccacatctggtgggacgatgacaagagatacaaccccgctctgaagagccggctgacaatcagcaaggaccctagcagtaaccaggtgttcctgaagatcgcttccgtggacacagcagacatcgcaacatactattgcgtgcggatcggcggaagcagtggatacatggactactggggacagggaaccagcgtgaccgtgagcagtggtggaggtggatcaggtggaggtggatctggtggaggtggatctgacatcgtgctgacccagagcccagctagcttggcagtgagcctgggacagagggctaccatcagctgcagagcttcagagagcgtggacagctacggaaacagcttcatgcactggtaccagcagaagccaggacagccacctaagctgctgatctaccgggctagcaacctgaagtccggaatccctgctcggtttagcggaagcggtagcggcaccgacttcaccctgacaatcaacccagtggaggccgacgatgtggcaacctactgctgtcagcagagcaacgaggacccatggaccttcggcggtggaaccaaactggagatcaagaga

SEQ ID NO: 57 - DNA, 729 bp, Artificial Sequence
  Description of Artificial Sequence Synthetic polynucleotide
caggtgaccctgaaggagtccggccccggcatcctgcagcccagccagaccctgagcctgacctgctccttcagcggcttctccctgtccacctccggcatgggcgtgggctggatcagacagcccagcggcaagggcctggagtggctggcccacatctggtgggacgatgacaagagatacaaccccgctctgaagagccggctgacaatcagcaaggaccctagcagtaaccaggtgttcctgaagatcgcttccgtggacacagcagacatcgcaacatactattgcgtgcggatcggcggaagcagtggatacatggactactggggacagggaaccagcgtgaccgtgagcagtggtggaggtggatcaggtggaggtggatctggtggaggtggatctaacatcgtgatgacccagtcccctaagagcatgagcatgagcgtgggcgagagagtgaccctgacctgcaaagcctccgagaacgtggtgacctacgtgagctggtaccagcagaagcctgagcagagccctaagctgctgatctacggcgcttccaacagatacaccggagtgcctgacagattcaccggcagcggaagcgcaaccgacttcaccttgaccatcagcagcgtgcaggctgaggacctggccgactaccactgcggccagggctacagctacccttacaccttcggtggaggcaccaagctggagatcaagcgg

SEQ ID NO: 58 - protein, 10 aa, Artificial Sequence
  Description of Artificial Sequence Synthetic peptide
EQKLISEEDL

SEQ ID NO: 59 - protein, 157 aa, Artificial Sequence
  Description of Artificial Sequence Synthetic polypeptide
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKP

SEQ ID NO: 60 - protein, 132 aa, Artificial Sequence
  Description of Artificial Sequence Synthetic polypeptide
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPW

SEQ ID NO: 61 - DNA, 471 bp, Artificial Sequence
  Description of Artificial Sequence Synthetic polynucleotide
atgagagccctgctggcgcgcctgcttctctgcgtcctggtcgtgagcgactccaaaggcagcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcgcagatggaaaaaagccc

SEQ ID NO: 62 - DNA, 396 bp, Artificial Sequence
  Description of Artificial Sequence Synthetic polynucleotide
atgagagccctgctggcgcgcctgcttctctgcgtcctggtcgtgagcgactccaaaggcagcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctgg

SEQ ID NO: 63 - protein, 4 aa, Homo sapiens
YVKM

SEQ ID NO: 64 - protein, 6 aa, Artificial Sequence
  Description of Artificial Sequence Synthetic 6xHis tag
HHHHHH

(19) United States
(12) Patent Application Publication
(10) Pub. No.: US 2022/0098320 A1
(43) Pub. Date: Mar. 31, 2022

(54) SENOLYTIC CAR-T CELLS TARGETING UPAR, A CELL SURFACE AND SECRETED SENESCENCE BIOMARKER

(71) Applicant: MEMORIAL SLOAN KETTERING CANCER CENTER, New York, NY (US)

(72) Inventors: Michel SADELAIN, New York, NY (US); Scott LOWE, New York, NY (US); Josef LEIBOLD, New York, NY (US); Corina AMOR, New York, NY (US); Judith FEUCHT, New York, NY (US)

(21) Appl. No.: 17/426,728

(22) PCT Filed: Jan. 31, 2020
(86) PCT No.: PCT/US2020/016290

§ 371 Date: Jul. 29, 2021

Related U.S. Application Data
(60) Provisional application No. 62/800,188, filed on Feb. 1, 2019.

(51) Int. Cl. C07K 16/28; A61K 35/17; A61P 35/02; A61P 35/00; A61P 1/16; G01N 33/574; G01N 33/68; A61K 45/06; A61K 39/395; C07K 14/705; C07K 14/725; A61K 38/17

(52) U.S. Cl. CPC C07K 16/2896; A61K 2039/505; A61P 35/02; A61P 35/00; A61P 1/16; G01N 33/57492; G01N 33/6896; G01N 33/6893; A61K 45/06; A61K 39/3955; C07K 14/70521; C07K 14/7051; A61K 38/1774; C07K 2317/565; A61K 35/17

(57) ABSTRACT

Provided herein are compositions and methods for adoptive cell therapy comprising engineered immune cells that express a uPAR-specific chimeric antigen receptor. Also disclosed herein are methods for using the engineered immune cells of the present technology to treat or ameliorate the effects of cancer and senescence-associated pathologies (e.g., lung fibrosis, atherosclerosis, Alzheimer's disease, diabetes, liver fibrosis, chronic kidney disease, aging, or osteoarthritis) by selectively targeting senescent cells. Also provided herein are methods for detecting the senescent cell burden in a patient.

### CROSS-REFERENCE TO RELATED APPLICATIONS

This application is a U.S. National Stage Application under 35 U.S.C § 371 of International Patent Application No. PCT/US20/016290, filed on Jan. 31, 2020, which claims the benefit of and priority to U.S. Provisional Application No. 62/800,188, filed Feb. 1, 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 28, 2020, is named 115872-0491_SL.txt and is 60,393 bytes in size.

### TECHNICAL FIELD

The present technology relates generally to compositions including engineered immune cells that express a uPAR-specific chimeric antigen receptor, and uses thereof.

### BACKGROUND

The following description of the background of the present technology is provided simply as an aid in understanding the present technology and is not admitted to describe or constitute prior art to the present technology.

Senescence is a stress response that limits tumor development and is lost during progression. At the same time, the aberrant accumulation of senescent cells has been linked to a variety of pathologies associated with chronic tissue damage or age, including fibrosis, atherosclerosis, and Alzheimer's disease, and experimental or pharmacological elimination of these cells has shown the ability to ameliorate some of these pathologies and extend lifespan in mice (He, S. & N. E. Sharpless, Cell 169(6): p. 1000-1011 (2017); Xu, M., et al., Nat Med 24(8): p. 1246-1256 (2018); Baar, M. P., et al., Cell, 169(1): p. 132-147 e16 (2017); Baker, D. J., et al., Nature, 479(7372): p. 232-6 (2011); Childs, B. G., et al., Science 354(6311): p. 472-477 (2016); Childs, B. G. et al., Nat Med, 21(12): p. 1424-35 (2015); Lasry, A. & Y. Ben-Neriah, Trends Immunol, 36(4): p. 217-28 (2015); Munoz-Espin, D., et al., EMBO Mol Med, 10(9) (2018)). In addition to the stable cell cycle arrest, senescent cells secrete an array of immune-cell attracting cytokines, chemokines, adhesion molecules, and metalloproteases collectively termed the “SASP” (Senescence-Associated Secretory Phenotype). The composition of the SASP as well as the surface proteins specifically upregulated in the membrane of senescent cells is heterogeneous and dependent on cell type as well as on the nature of the senescence trigger. See Lasry & Ben-Neriah, Trends Immunol. 36; 217-228 (2015); Kim et al., Genes and Dev. 31; 1529-1534 (2017); see also Table A.

TABLE A

| | OIS in vitro | OIS in vivo | DIS |
|---|---|---|---|
| AREG(Amphiregulin) | 8.94 | 4.24 | 0.00 |
| AXL | 0.00 | 0.00 | 0.00 |
| CD9 | 2.26 | 1.75 | 0.00 |
| CSF2 | 0.00 | 0.00 | 0.00 |
| DPP4 | 1.76 | 0.00 | 0.00 |
| Epiregulin (EREG) | 6.34 | 4.74 | 0.00 |
| ETS2 | 1.72 | 0.00 | 0.00 |
| FAS | 0.00 | 0.00 | 3.08 |
| GEM | 0.00 | 0.00 | 0.00 |
| ICAM-1 | 3.89 | 0.00 | 3.03 |
| IGF-1 | 0.00 | 0.00 | 0.00 |

OIS = oncogene-induced senescence;
DIS = drug-induced senescence

Thus, to date, a common cell surface marker of senescence has not been identified. Accordingly, the identification of reliable and universal senescence-specific molecular biomarkers, regardless of cell type and senescence triggers, is critical for the detection of senescent cells in tissues, and the development of effective therapeutics towards these pathologies.

### SUMMARY OF THE PRESENT TECHNOLOGY

Provided herein, in certain embodiments, are compositions and methods for adoptive cell therapy comprising engineered immune cells that express a receptor that binds to a uPAR antigen.

In one aspect, the present disclosure provides an engineered immune cell including a receptor that comprises a uPAR antigen binding fragment comprising: a V_{H}CDR1 sequence, a V_{H}CDR2 sequence, and a V_{H}CDR3 sequence of GFSLSTSGM (SEQ ID NO: 35), WWDDD (SEQ ID NO: 36), and IGGSSGYMDY (SEQ ID NO: 37), respectively; and/or a V_{L}CDR1 sequence, a V_{L}CDR2 sequence, and a V_{L}CDR3 sequence of: RASESVDSYGNSFMH (SEQ ID NO: 41), RASNLKS (SEQ ID NO: 42), and QQSNEDPWT (SEQ ID NO: 43) respectively; or KASENVVTYVS (SEQ ID NO: 44), GASNRYT (SEQ ID NO: 45), and GQGYSYPYT (SEQ ID NO: 46), respectively, and/or a nucleic acid encoding the receptor. The uPAR antigen binding fragment may comprise a V_{H} amino acid sequence of SEQ ID NO: 48 and/or a V_{L} amino acid sequence of SEQ ID NO: 50 or SEQ ID NO: 51.

In another aspect, the present disclosure provides an engineered immune cell including a receptor that comprises a uPAR antigen binding fragment comprising an amino acid sequence selected from the group consisting of: SEQ ID NO: 52, SEQ ID NO: 53, and SEQ ID NO: 54; and/or a nucleic acid encoding the receptor (e.g., SEQ ID NO: 55, SEQ ID NO: 56, and SEQ ID NO: 57).

In one aspect, the present disclosure provides an engineered immune cell including a chimeric antigen receptor that comprises a uPAR antigen binding fragment comprising: a V_{H}CDR1 sequence, a V_{H}CDR2 sequence, and a V_{H}CDR3 sequence of GFTFSNY (SEQ ID NO: 32), STGGGN (SEQ ID NO: 33), and QGGGYSDSFDY (SEQ ID NO:34), respectively, and a V_{L}CDR1 sequence, a V_{L}CDR2 sequence, and a V_{L}CDR3 sequence of KASKSISKYLA (SEQ ID NO: 38), SGSTLQS (SEQ ID NO: 39), and QQHNEYPLT (SEQ ID NO: 40), respectively, and/or a nucleic acid encoding the receptor. The uPAR antigen binding fragment may comprise a V_{H} amino acid sequence of SEQ ID NO: 47 and/or a V_{L} amino acid sequence of SEQ ID NO: 49.

In any of the embodiments of the engineered immune cells disclosed herein, the receptor is a T cell receptor. The receptor may be a non-native receptor (e.g., a non-native T cell receptor), for example, an engineered receptor, such as a chimeric antigen receptor (CAR). Additionally or alternatively, in some embodiments of the engineered immune cells of the present technology, the anti-uPAR antigen binding fragment is an scFv, a Fab, or a (Fab)₂. Additionally or alternatively, in some embodiments of the engineered immune cells of the present technology, the receptor may be linked to a reporter or a selection marker (e.g., GFP or LNGFR). In certain embodiments, the receptor is linked to the reporter or selection marker via a self-cleaving linker. In some embodiments, the self-cleaving peptide is a P2A self-cleaving peptide.

Additionally or alternatively, in some embodiments, the engineered immune cell is a lymphocyte, such as a T-cell, a B cell or a natural killer (NK) cell, or a tumor infiltrating lymphocyte. In some embodiments, the T cell is a CD4⁺ T cell or a CD8⁺ T cell. In some embodiments, the engineered immune cell is derived from an autologous donor or an allogenic donor.

Additionally or alternatively, in some embodiments, the engineered immune cells comprise a chimeric antigen receptor and/or nucleic acid encoding the chimeric antigen receptor, wherein the chimeric antigen receptor comprises (i) an extracellular antigen binding domain; (ii) a transmembrane domain; and (iii) an intracellular domain. In some embodiments, the extracellular antigen binding domain binds to a uPAR antigen.

Additionally or alternatively, in some embodiments, the extracellular antigen binding domain of the chimeric antigen receptor comprises a single chain variable fragment (scFv). In some embodiments, the extracellular antigen binding domain of the chimeric antigen receptor comprises a human scFv. Additionally or alternatively, in some embodiments, the extracellular antigen binding domain of the chimeric antigen receptor comprises a uPAR antigen binding fragment (e.g., an scFv) comprising an amino acid sequence selected from the group consisting of: SEQ ID NO: 52, SEQ ID NO: 53, and SEQ ID NO: 54. Additionally or alternatively, in some embodiments, the extracellular antigen binding domain of the chimeric antigen receptor comprises a uPAR antigen binding fragment (e.g., an scFv) having at least 80%, 85%, 90%, 95%, 96%, 97%, 98%, or 99% sequence identity to any one of SEQ ID NOs: 52-54.

Additionally or alternatively, in some embodiments, the extracellular antigen binding domain of the chimeric antigen receptor comprises a signal peptide (e.g., a CD8 signal peptide) that is covalently joined to the N-terminus of the extracellular antigen binding domain. Additionally or alternatively, in some embodiments, the transmembrane domain of the chimeric antigen receptor comprises a CD8 transmembrane domain or a CD28 transmembrane domain. Additionally or alternatively, in some embodiments, the intracellular domain of the chimeric antigen receptor comprises one or more costimulatory domains. The one or more costimulatory domains may be selected from among a CD28 costimulatory domain, a 4-1BB costimulatory domain, an OX40 costimulatory domain, an ICOS costimulatory domain, a DAP-10 costimulatory domain, a PD-1 costimulatory domain, a CTLA-4 costimulatory domain, a LAG-3 costimulatory domain, a 2B4 costimulatory domain, a BTLA costimulatory domain, a CD3ζ-chain, or any combination thereof.

Additionally or alternatively, in some embodiments, the nucleic acid encoding the receptor is operably linked to a promoter. The promoter may be a constitutive promoter or a conditional promoter. In some embodiments, the conditional promoter is inducible by binding of the receptor (e.g., a CAR) to a uPAR antigen.

Also disclosed herein are polypeptides comprising a uPAR-specific chimeric antigen receptor comprising an amino acid sequence of any one of SEQ ID NOs: 47, 48, 49, and 50-54, and optionally a reporter or a selection marker (e.g., GFP, LNGFR). In some embodiments, the polypeptides further comprise a self-cleaving peptide located between the uPAR-specific chimeric antigen receptor and the reporter or selection marker (e.g., GFP, LNGFR). In certain embodiments, the self-cleaving peptide is a P2A self-cleaving peptide. Additionally or alternatively, in some embodiments, the uPAR-specific chimeric antigen receptor further comprises a leader sequence. The leader sequence may comprise an amino acid sequence of SEQ ID NO: 3, SEQ ID NO: 5, SEQ ID NO: 7 or SEQ ID NO: 9. Additionally or alternatively, in some embodiments, the uPAR-specific chimeric antigen receptor comprises (i) an extracellular antigen binding domain; (ii) a transmembrane domain; and (iii) an intracellular domain. In some embodiments, the extracellular antigen binding domain of the chimeric antigen receptor binds to a uPAR antigen. Additionally or alternatively, in some embodiments, the extracellular antigen binding domain of the chimeric antigen receptor comprises a single chain variable fragment (scFv). In some embodiments, the extracellular antigen binding domain of the chimeric antigen receptor comprises a uPAR scFv of any one of SEQ ID NOs: 52-54. In other embodiments, the extracellular antigen binding domain of the chimeric antigen receptor comprises a uPAR scFv having at least 80%, 85%, 90%, 95%, 96%, 97%, 98%, or 99% sequence identity to any one of SEQ ID NOs: 52-54.

Additionally or alternatively, in some embodiments of the polypeptides disclosed herein, the transmembrane domain of the chimeric antigen receptor comprises a CD8 transmembrane domain or a CD28 transmembrane domain. Additionally or alternatively, in some embodiments of the polypeptides disclosed herein, the intracellular domain of the chimeric antigen receptor comprises one or more costimulatory domains. The one or more costimulatory domains may be selected from among a CD28 costimulatory domain, a 4-1BB costimulatory domain, an OX40 costimulatory domain, an ICOS costimulatory domain, a DAP-10 costimulatory domain, a PD-1 costimulatory domain, a CTLA-4 costimulatory domain, a LAG-3 costimulatory domain, a 2B4 costimulatory domain, a BTLA costimulatory domain, a CD3ζ-chain, or any combination thereof.

Also provided herein are nucleic acids encoding any of the polypeptides disclosed herein. In some embodiments, the nucleic acid encoding the polypeptide is operably linked to a promoter. The promoter may be a constitutive promoter or a conditional promoter. In some embodiments, the conditional promoter is inducible by the chimeric antigen receptor binding to a uPAR antigen. Also provided herein are vectors comprising any of the nucleic acids disclosed herein. In some embodiments, the vector is a viral vector or a plasmid. In some embodiments, the vector is a retroviral vector.

Also disclosed herein are host cells comprising a polypeptide, a nucleic acid, or a vector disclosed herein.

Also provided are methods for treating cancer in a subject in need thereof comprising administering to the subject an effective amount of any of the engineered immune cells provided herein, wherein the subject is receiving/has received senescence-inducing therapies (e.g., chemotherapeutic agents). In some embodiments, the methods further comprise administering to the subject a tumor specific monoclonal antibody. In some embodiments, the tumor specific monoclonal antibody is administered subsequent to administration of the engineered immune cells. Also provided herein are methods for inhibiting tumor growth or metastasis in a subject in need thereof comprising contacting a tumor cell with an effective amount of any of the engineered immune cells provided herein. In some embodiments, the methods further comprise administering to the subject a tumor specific monoclonal antibody. In some embodiments, the tumor specific monoclonal antibody is administered subsequent to administration of the engineered immune cells.

Additionally or alternatively, in some embodiments of the methods disclosed herein, the engineered immune cell(s) are administered are administered intravenously, intratumorally, intraperitoneally, subcutaneously, intramuscularly, or intratumorally. In some embodiments, the cancer or tumor is selected from among breast cancer, endometrial cancer, ovarian cancer, colon cancer, lung cancer, stomach cancer, prostate cancer, renal cancer, pancreatic cancer, acute lymphoblastic leukemia (ALL), chronic lymphocytic leukemia (CLL), acute myeloid leukemia (AMIL), and metastases thereof.

Additionally or alternatively, in some embodiments, the methods of the present technology further comprise administering to the subject an additional cancer therapy. In some embodiments, the additional cancer therapy is selected from among chemotherapy, radiation therapy, immunotherapy, monoclonal antibodies, anti-cancer nucleic acids or proteins, anti-cancer viruses or microorganisms, and any combinations thereof. In some embodiments, the methods further comprise administering a cytokine to the subject. In some embodiments, the cytokine is administered prior to, during, or subsequent to administration of the one or more engineered immune cells. In some embodiments, the cytokine is selected from the group consisting of interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

The methods for treating cancer may further comprise sequentially, separately, or simultaneously administering to the subject at least one chemotherapeutic agent selected from the group consisting of nitrogen mustards, ethylenimine derivatives, alkyl sulfonates, nitrosoureas, gemcitabine, triazenes, folic acid analogs, anthracyclines, taxanes, COX-2 inhibitors, pyrimidine analogs, purine analogs, antibiotics, enzyme inhibitors, epipodophyllotoxins, platinum coordination complexes, vinca alkaloids, substituted ureas, methyl hydrazine derivatives, adrenocortical suppressants, hormone antagonists, endostatin, taxols, camptothecins, SN-38, doxorubicin, doxorubicin analogs, antimetabolites, alkylating agents, antimitotics, anti-angiogenic agents, tyrosine kinase inhibitors, mTOR inhibitors, heat shock protein (HSP90) inhibitors, proteosome inhibitors, HDAC inhibitors, pro-apoptotic agents, methotrexate and CPT-11. In some embodiments, the subject is human.

Also provided are methods for preparing immune cells for therapy, comprising isolating immune cells from a donor subject, transducing the immune cells (e.g., T cells) with (a) a nucleic acid provided herein, or (b) a vector provided herein. In some embodiments, the immune cells isolated from the donor subject comprise one or more lymphocytes. In some embodiments, the lymphocytes comprise a T-cell, a B cell, and/or a natural killer (NK) cell. In some embodiments, the T cell is a CD4⁺ T cell or a CD8⁺ T cell. In some embodiments, the immune cells isolated from the donor subject comprise tumor infiltrating lymphocytes (TILs).

Also provided are methods for treatment comprising isolating immune cells from a donor subject, transducing the immune cells with (a) a nucleic acid provided herein, or (b) a vector provided herein, and administering the transduced immune cells to a recipient subject. In some embodiments, the donor subject and the recipient subject are the same (i.e., autologous). In some embodiments, the donor subject and the recipient subject are different (i.e., allogenic). In some embodiments, the immune cells isolated from the donor subject comprise one or more lymphocytes. In some embodiments, the lymphocytes comprise a T-cell, a B cell, and/or a natural killer (NK) cell. In some embodiments, the T cell is a CD4⁺ T cell or a CD8⁺ T cell. In some embodiments, the immune cells isolated from the donor subject comprise tumor infiltrating lymphocytes (TILs).

Also disclosed herein are kits comprising at least one engineered immune cell of the present technology, and instructions for use. In another aspect, the present disclosure provides kits comprising reagents for detecting uPAR/suPAR expression levels in a biological sample obtained from a subject, and instructions for detecting the presence of senescent cells (e.g., SASP) in the sample.

In another aspect, the present disclosure provides methods for treating or ameliorating the effects of a senescence-associated pathology in a subject in need thereof comprising administering to the subject an effective amount of any of the engineered immune cells described herein, wherein the subject exhibits an increased accumulation of senescent cells compared to that observed in a healthy control subject. In some embodiments, the senescence-associated pathology is lung fibrosis, atherosclerosis, Alzheimer's disease, diabetes, liver fibrosis, chronic kidney disease, aging, or osteoarthritis. Additionally or alternatively, in certain embodiments, the senescent cells exhibit a Senescence-Associated Secretory Phenotype (SASP). The Senescence-Associated Secretory Phenotype may be induced by an oncogene (e.g., HRAS^{G12D}, NRAS^{G12D}, NRAS^{G12D; D38A} etc.) or a drug (e.g., Cdk4/6 inhibitors (e.g., palbociclib), MEK inhibitors (e.g., trametinib), doxorubicin). Additionally or alternatively, in some embodiments, the methods further comprise sequentially, separately, or simultaneously administering to the subject at least one additional agent selected from the group consisting of statins (e.g., Atorvastatin, Fluvastatin, Lovastatin, Pitavastatin, Pravastatin, Rosuvastatin calcium, Simvastatin), fibrates (e.g., Gemfibrozil, Fenofibrate), niacin, ezetimibe, bile acid sequestrants (e.g., cholestyramine, colestipol, colesevelam), proprotein convertase subtilisin kexin type 9 (PCSK9) inhibitors, anti-platelet medications (e.g., aspirin, Clopidogrel, Ticagrelor, warfarin, prasugral), beta blockers, Angiotensin-converting enzyme (ACE) inhibitors (e.g., benazepril, Lisinopril, Ramipril), calcium channel blockers, diuretics, donepezil, galantamine, memantine, rivastigmine, memantine extended-release and donepezil (Namzaric), aducanumab, solanezumab, insulin, verubecestat, AADvac1, CSP-1103, intepirdine, insulin, metformin, amylin analogs, glucagon, sulfonylureas (e.g., glimepiride, glipizide, glyburide, chlorpropamide, tolazamide, tolbutamide), meglitinides (e.g., nateglinide, repaglinide), thiazolidinediones (e.g., pioglitazone, rosiglitazone), alpha-glucosidase inhibitors (e.g., acarbose, miglitol), dipeptidyl peptidase (DPP-4) inhibitors (e.g., alogliptin, linagliptin, sitagliptin, saxagliptin), sodium-glucose co-transporter 2 (SGLT2) inhibitors (e.g., canagliflozin, dapagliflozin, empagliflozin, ertugliflozin), incretin mimetics (e.g., exenatide, liraglutide, dulaglutide, lixisenatide, semaglutide), analgesics (e.g., acetaminophen, tramadol, oxycodone, hydrocodone), nonsteroidal anti-inflammatory drugs (e.g., aspirin, ibuprofen, naproxen, celecoxib), cyclooxygenase-2 inhibitors, corticosteroids, hyaluronic acid, α-Tocopherol, interferon-α, PPAR-antagonists, colchicine, endothelin inhibitors, interleukin-10, pentoxifylline, phosphatidylcholine, S-adenosyl-methionine, TGF-β1 inhibitors, furosemide, erythropoietin, phosphate binders (e.g., calcium acetate, calcium carbonate), colecalciferol, ergocalciferol, and cyclophosphamide.

In one aspect, the present disclosure provides a method for detecting senescent cells in a biological sample obtained from a patient comprising: detecting the presence of senescent cells in the biological sample by detecting uPAR and/or suPAR polypeptide levels in the biological sample that are increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 100% compared to that observed in a reference sample. Alternatively, the present disclosure provides a method for detecting senescent cells in a biological sample obtained from a patient comprising: detecting the presence of senescent cells in the biological sample by detecting uPAR and/or suPAR polypeptide levels in the biological sample that are increased by at least 0.5-fold, at least 1.0 fold, at least 1.5-fold, at least 2.0 fold, at least 2.5-fold, at least 3.0 fold, at least 3.5-fold, at least 4.0 fold, at least 4.5-fold, at least 5.0 fold, at least 5.5-fold, at least 6.0 fold, at least 6.5-fold, at least 7.0 fold, at least 7.5-fold, at least 8.0 fold, at least 8.5-fold, at least 9.0 fold, at least 9.5-fold, or at least 10.0 fold compared to that observed in a reference sample. The reference sample may be obtained from a healthy control subject or may contain a predetermined level of the uPAR and/or suPAR polypeptide. The biological sample may be mucus, saliva, bronchial alveolar lavage (BAL), bronchial wash (BW), whole blood, cerebrospinal fluid (CSF), urine, plasma, serum, lymph, semen, synovial fluid, tears, amniotic fluid, bile, aqueous humor, or a bodily fluid. Additionally or alternatively, in some embodiments, the uPAR and/or suPAR polypeptide levels are detected via Western Blotting, flow cytometry, Enzyme-linked immunosorbent assay (ELISA), immunoprecipitation, immunoelectrophoresis, immunostaining, isoelectric focusing, High-performance liquid chromatography (HPLC), or mass-spectrometry.

In one aspect, the present disclosure provides a method for determining the efficacy of a senescence-inducing therapy in a patient in need thereof comprising: detecting uPAR and/or soluble uPAR (suPAR) polypeptide levels in a test biological sample obtained from the patient after administration of the senescence-inducing therapy, wherein the senescence-inducing therapy is effective when the uPAR and/or suPAR polypeptide levels in the test biological sample are elevated compared to that observed in a control biological sample obtained from the patient prior to administration of the senescence-inducing therapy. In some embodiments, the patient is suffering from or has been diagnosed with a senescence-associated pathology such as cancer, lung fibrosis, atherosclerosis, Alzheimer's disease, diabetes, osteoarthritis, liver fibrosis, or chronic kidney disease. Additionally or alternatively, in some embodiments, the senescence-inducing therapy includes the use of a chemotherapeutic agent and/or a targeted immunotherapy. Additionally or alternatively, in some embodiments, the method further comprises selecting the patient for treatment with an engineered immune cell that specifically targets uPAR (e.g., CAR T cells of the present technology) when the uPAR and/or suPAR polypeptide levels in the test biological sample are elevated compared to that observed in the control biological sample.

In another aspect, the present disclosure provides a method for determining the efficacy of a senolytic CAR T cell therapy in a patient in need thereof comprising: detecting uPAR and/or soluble uPAR (suPAR) polypeptide levels in a test biological sample obtained from the patient after administration of the senolytic CAR T cell therapy, wherein the senolytic CAR T cell therapy is effective when the uPAR and/or suPAR polypeptide levels in the test biological sample are reduced compared to that observed in a control biological sample obtained from the patient prior to administration of the senolytic CAR T cell therapy. In some embodiments, the patient is suffering from or has been diagnosed with a senescence-associated pathology such as cancer, lung fibrosis, atherosclerosis, Alzheimer's disease, diabetes, osteoarthritis, liver fibrosis, or chronic kidney disease.

In yet another aspect, the present disclosure provides a method for selecting patients affected by a senescence-associated pathology for treatment with senolytic CAR T cell therapy comprising: (a) detecting uPAR and/or soluble uPAR (suPAR) polypeptide levels in biological samples obtained from the patients; (b) identifying patients that exhibit uPAR and/or soluble uPAR (suPAR) polypeptide levels that are elevated by at least 5% compared to a predetermined threshold; and (c) administering an engineered immune cell that specifically targets uPAR to the patients of step (b). The senescence-associated pathology may be cancer, lung fibrosis, atherosclerosis, Alzheimer's disease, diabetes, osteoarthritis, liver fibrosis, or chronic kidney disease. In some embodiments, the engineered immune cell that specifically targets uPAR is any engineered immune cell disclosed herein. Additionally or alternatively, in some embodiments, the uPAR and/or suPAR polypeptide levels are detected via Western Blotting, flow cytometry, Enzyme-linked immunosorbent assay (ELISA), immunoprecipitation, immunoelectrophoresis, immunostaining, isoelectric focusing, High-performance liquid chromatography (HPLC), or mass-spectrometry. In any of the preceding embodiments of the methods disclosed herein, the biological samples comprise mucus, saliva, bronchial alveolar lavage (BAL), bronchial wash (BW), whole blood, cerebrospinal fluid (CSF), urine, plasma, serum, lymph, semen, synovial fluid, tears, amniotic fluid, bile, aqueous humor, or bodily fluids.

Also disclosed herein are methods for treating or ameliorating the effects of a senescence-associated pathology in a subject in need thereof comprising administering to the subject an effective amount of an engineered immune cell, wherein the engineered immune cell includes a receptor that comprises the amino acid of SEQ ID NO: 59 or SEQ ID NO: 60, and/or a nucleic acid encoding the receptor (e.g., SEQ ID NO: 61 or SEQ ID NO: 62), wherein the subject exhibits an increased accumulation of senescent cells compared to that observed in a healthy control subject. The senescence-associated pathology may be lung fibrosis, atherosclerosis, Alzheimer's disease, diabetes, osteoarthritis, liver fibrosis, chronic kidney disease, breast cancer, endometrial cancer, colon cancer, lung cancer, stomach cancer, prostate cancer, renal cancer, pancreatic cancer, acute lymphoblastic leukemia (ALL), chronic lymphocytic leukemia (CLL), acute myeloid leukemia (AML), and metastases thereof. In any of the embodiments of the methods disclosed herein, the receptor is a T cell receptor. The receptor may be a non-native receptor (e.g., a non-native T cell receptor), for example, an engineered receptor, such as a chimeric antigen receptor (CAR). Additionally or alternatively, in some embodiments of the methods of the present technology, the receptor may be linked to a reporter or a selection marker (e.g., GFP or LNGFR). In certain embodiments, the receptor is linked to the reporter or selection marker via a self-cleaving linker. In some embodiments, the self-cleaving peptide is a P2A self-cleaving peptide.

Additionally or alternatively, in some embodiments, the engineered immune cell is a lymphocyte, such as a T-cell, a B cell or a natural killer (NK) cell, or a tumor infiltrating lymphocyte. In some embodiments, the T cell is a CD4⁺ T cell or a CD8⁺ T cell. In some embodiments, the engineered immune cell is derived from an autologous donor or an allogenic donor.

Additionally or alternatively, in some embodiments of the methods disclosed herein, the chimeric antigen receptor comprises (i) an extracellular uPA fragment that is configured to bind to a uPAR polypeptide; (ii) a transmembrane domain; and (iii) an intracellular domain. The extracellular uPA fragment may comprise a human uPA fragment. In certain embodiments, the extracellular uPA fragment comprises the amino acid sequence of SEQ ID NO: 59 or SEQ ID NO: 60. In other embodiments, the extracellular uPA fragment comprises an amino acid sequence that is at least 80%, 85%, 90%, 95%, 96%, 97%, 98%, or 99% identical to SEQ ID NO: 59 or SEQ ID NO: 60. In any and all of the preceding embodiments of the methods disclosed herein, the extracellular uPA fragment of the chimeric antigen receptor comprises a signal peptide (e.g., a CD8 signal peptide) that is covalently joined to the N-terminus of the extracellular uPA fragment.

Additionally or alternatively, in some embodiments of the methods disclosed herein, the transmembrane domain of the chimeric antigen receptor comprises a CD8 transmembrane domain or a CD28 transmembrane domain. Additionally or alternatively, in some embodiments, the intracellular domain of the chimeric antigen receptor comprises one or more costimulatory domains. The one or more costimulatory domains may be selected from among a CD28 costimulatory domain, a 4-1BB costimulatory domain, an OX40 costimulatory domain, an ICOS costimulatory domain, a DAP-10 costimulatory domain, a PD-1 costimulatory domain, a CTLA-4 costimulatory domain, a LAG-3 costimulatory domain, a 2B4 costimulatory domain, a BTLA costimulatory domain, a CD3ζ-chain, or any combination thereof.

Additionally or alternatively, in some embodiments of the methods, the nucleic acid encoding the receptor is operably linked to a promoter. The promoter may be a constitutive promoter or a conditional promoter. In some embodiments, the conditional promoter is inducible by binding of the receptor to a uPAR polypeptide.

In one aspect, the present disclosure provides a method for treating cancer in a subject in need thereof comprising administering to the subject an effective amount of any engineered immune cell disclosed herein, and an effective amount of a senescence-inducing agent. In some embodiments, the senescence-inducing agent is doxorubicin, ionizing radiation therapy, combination therapy with a MEK inhibitor and a CDK4/6 inhibitor, or combination therapy with a CDCl₇ inhibitor and a mTOR inhibitor. Examples of MEK inhibitors include, but are not limited to PD-325901, TAK-733, CI-1040 (PD184352), PD0325901, MEK162, AZD8330, GDC-0623, refametinib, pimasertib, RO4987655, RO5126766, WX-554, HL-085, CInQ-03, G-573, PD184161, PD318088, PD98059, RO5068760, U0126, and SL327. Examples of CDK4/6 inhibitors include, but are not limited to palbociclib, ribociclib, and abemaciclib. Examples of CDCl₇ inhibitors include, but are not limited to, TAK-931, PHA-767491, XL413, 1H-pyrrolo[2,3-b]pyridines, 2,3-dihydrothieno[3,2-d]pyrimidin-4(1H)-ones, furanone derivatives, and trisubstituted thiazoles, pyrrolopyridinones. Examples of mTOR inhibitors include, but are not limited to, rapamycin, sertraline, sirolimus, everolimus, temsirolimus, ridaforolimus, and deforolimus.

### BRIEF DESCRIPTION OF THE DRAWINGS

FIG. 1A shows the heatmap of genes upregulated upon therapy-induced senescence (TIS), oncogene-induced senescence (OIS) or p53 induced senescence in hepatic stellate cells (HSCs). FIG. 1B shows a Venn diagram displaying the number of common genes upregulated in the three databases shown in FIG. 1A. FIG. 1C shows the combined enrichment score of significantly enriched gene sets among the 8 commonly upregulated genes in senescence.

FIG. 2A shows the flow cytometric analysis comparing human uPAR (h.uPAR) expression in primary human melanocytes induced to senescence by continuous passage with proliferating controls and representative SA-β-Gal staining of the cells. Representative results of two independent experiments, including fluorescence minus one (FMO) controls are shown. FIG. 2B shows the qRT-PCR analysis of SASP gene expression in senescent (passage 15) versus proliferating (passage 2) primary human melanocytes. Representative results of two independent experiments are shown. FIG. 2C shows the flow cytometric analysis of the mouse uPAR (m.uPAR) expression in Kras^{G12D};p53^{-/-} murine lung adenocarcinoma cells (KP) induced to senescence by combined treatment with MEK (25 nM) and Cdk4/6 (500 nM) inhibitors (MEKi, Cdk4/6i) for 8 days as compared to controls and representative senescence-associated beta-galactosidase (SA-β-Gal) staining of the cells. Representative results of four independent experiments are shown. FIG. 2D shows the quantitative reverse transcription polymerase chain reaction (qRT-PCR) analysis of Senescence-Associated Secretory Phenotype (SASP) gene expression in senescent versus proliferating KP tumor cells. Representative results of two independent experiments are shown. FIG. 2E shows the representative immunohistochemical stainings of human uPAR and SA-β-Gal of a patient-derived xenograft (PDX) from human lung adenocarcinoma orthotopically injected into NSG mice. The immunohistochemical staining was performed after treatment with either vehicle or combined MEK (1 mg/kg body weight) and Cdk4/6 inhibitors (100 mg/kg body weight). Representative results of two independent experiments (n=3 mice per group) are shown. For FIG. 2B, two-tailed unpaired Student's t-test was used. For FIG. 2B, data represent mean±SD. FIG. 2F shows the levels of soluble uPAR (suPAR) (pg/ml) as determined by ELISA in the supernatant of senescent or proliferating KP cells. Representative results of two independent experiments are shown. FIG. 2G shows the levels of suPAR (pg/ml) as determined by ELISA in the supernatant of senescent (Passage 15=P.15) or proliferating (Passage 2=P.2) human primary melanocytes. Representative results of two independent experiments are shown.

FIG. 3A shows the expression of uPAR (as determined by IHC) in murine PanINs (senescent lesions) but not in acute injury (cerulein at day 2 and cerulein and KRAS^{G12D} at day 2). FIG. 3B shows the co-immunofluorescence staining for KATE (red marking p48Cre-recombined pancreatic epithelial cells) and murine uPAR (green) in the pancreas of mice either expressing endogenous KRAS^{G12D} and 21 weeks after treatment with cerulein to induce pancreatic intraepithelial neoplasia (PanIN) or mice with normal pancreas (KRAS WT). Representative results of two independent experiments (n=3 mice per group) are shown. FIG. 3C shows the expression of uPAR (as determined by IHC) in human PanINs. FIG. 3D shows the expression of uPAR (as determined by IHC) in murine liver in NRAS^{G12V}-induced senescence (hydrodynamic tail vein injection (HTVI) model). FIG. 3E shows the co-immunofluorescence staining for murine uPAR (red) and NRAS (green) in the livers of mice 6 days after hydrodynamic tail vein injection (HTVI) of a plasmid encoding NRAS^{G12D} or the GTPase dead version NRAS^{G12D;D38A}. Representative results of three independent experiments (n=5 mice per group) are shown. FIG. 3F shows the co-immunofluorescence stainings for murine uPAR (m.uPAR in red) and ki-67 (green) or murine uPAR (red) and IL6 (green) in murine livers 6 days after hydrodynamic tail vein injection (HTVI) with a plasmid encoding NRAS^{G12D}. Immunohistochemical stainings of murine uPAR or P-ERK in consecutive sections of the same liver 6 days after HTVI. Representative results of three independent experiments are shown.

FIGS. 4A-4B demonstrate the up-regulation of uPAR in senescence-associated diseases. FIG. 4A shows expression of uPAR (as determined by IHC) in a mouse model of lung fibrosis (treatment with intratracheal bleomycin 1 mg/kg) and IF showing co-localization between uPAR and smooth muscle actin in the fibrosis foci. FIG. 4B shows the expression of human uPAR in atherosclerotic plaques (specimens obtained from endarterectomy). FIG. 4C shows the immunohistochemical expression of human uPAR (h.uPAR) and SA-β-Gal in human samples of hepatitis-induced liver fibrosis (n=10 patients), immunohistochemical stainings for human uPAR (h.uPAR) in human carotid endarterectomy samples (n=5 patients) and in human pancreas bearing pancreatic intraepithelial neoplasia (PanINs) (n=3 patients).

FIG. 5A shows the co-immunofluorescence staining for murine uPAR (red) and smooth muscle actin (green) in the livers of mice 6 weeks after semi-weekly i.p. treatment with either CCl₄ or vehicle. Representative results of three independent experiments (n=4-7 mice per group). FIG. 5B shows fold change in the plasma levels of suPAR in mice that have received semi-weekly i.p treatments with either CCl₄ or vehicle for 6 weeks. Representative results of two independent experiments are shown. For FIG. 5B, all data are mean±SEM. Two-tailed unpaired Student's t-test. *P<0.05, **P<0.01.

FIG. 6A shows the expression profile of uPAR in the described cell types as determined by mass spectrometry. FIG. 6B shows the expression levels of uPAR in the different organs as determined by IHC. Expression in the bone marrow is restricted to monocytes and expression in the lung category corresponds to nasopharynx and epithelial layer of the bronchi, not to the lung parenchyma. FIG. 6C shows the immunohistochemical staining of murine uPAR (m.uPAR) in vital tissues of C57BL/6J mice. Representative results of two independent experiments are shown. FIG. 6D shows the heatmap showing the expression profile of human uPAR (PLAUR) in human vital tissues as determined by the Human Proteome Map (HPM) as compared to the expression profiles of other CAR targets in current clinical trials.

FIG. 7A shows the surface expression of uPAR (as determined by FACS) in KRAS^{G12D}; p53^{-/-} murine lung tumor cells lines (WT or uPAR CRISPR-KO) during MEKi and Palboi-induced senescence. FIG. 7B shows levels of suPAR in the media of KRAS^{G12D}; p53^{-/-} murine lung tumor cells lines (KP, without or with uPAR CRISPR-KO) during MEKi and Palboi-induced senescence. FIG. 7C shows a schematic representation of an in vivo doxorubicin-induced senescence model. FIG. 7D shows the experimental layout: C57BL/6J mice were intraperitoneally (i.p.) treated with either doxorubicin (10 mg/kg) or vehicle at day 0 and received a second dose of either agent 10 days after. Blood was drawn from the mice 10 and 20 days after the first injection. Levels of suPAR in the plasma of these mice at day 10 and at day 20 after treatment initiation (n=4 mice per group). Representative results of two independent experiments are shown (n=4 mice per group). FIG. 7E shows the immunohistochemical staining of murine uPAR in the livers and kidneys of vehicle or doxorubicin-treated mice harvested at day 20 after treatment initiation. Representative results of two independent experiments are shown (n=4 mice per group). For FIG. 7D, two-tailed unpaired Student's t-test was used. For FIG. 7D data represent mean±SEM. *P<0.05,**P<0.01, ***P<0.001.

FIGS. 8A-8D demonstrate that the serum suPAR levels reflect NRAS-induced senescence. FIG. 8A shows a schematic representation of the hydrodynamic tail vein injection of either NRAS^{G12V} or the GTPase dead mutant NRAS^{G12V; D38A}. FIG. 8B shows representative bright field and GFP images. FIG. 8C shows immunofluorescence (IF) images showing the expression of NRAS in the livers of mice that received either NRAS^{G12V} or NRAS^{G12V; D38A} whereas uPAR is only expressed in the NRAS^{G12V} (hepatocytes that underwent NRAS-induced senescence). FIG. 8D shows serum levels of suPAR.

FIGS. 9A-9E demonstrate that the serum suPAR levels correlate with lung fibrosis. FIG. 9A shows a schematic representation of the KC; RIK model of acinar-to-ductal metaplasia (ADM); acinar-to-ductal reprogramming (ADR); pancreatic intra-epithelial neoplasia (PanIN) as described in (Livshits et al, eLife 7:e35216 (2018)). FIG. 9B shows serum levels of suPAR in the KC; RIK model. FIG. 9C shows a schematic representation of the model of lung fibrosis. NSG mice were treated with intratracheal bleomycin (1 U/Kg) or PBS. FIG. 9D shows representative IHC images showing induction of fibrosis in the bleomycin treated cohort and upregulation of uPAR in the fibrotic foci. FIG. 9E shows serum levels of suPAR in the murine model of lung fibrosis.

FIG. 10A shows the construct maps encoding human h.uPAR-h.28z and h.CD19-h.28z CAR T cells and murine m.uPAR-m.28z and m.CD19-m.28z CARs. FIG. 10B shows a representative nucleotide sequence (SEQ ID NO: 55) of the anti-mouse uPAR scFv comprising a V_{H} domain, a GS linker and a V_{L} domain. FIG. 10C shows a representative amino acid sequence (SEQ ID NO: 52) of anti-mouse uPAR scFv comprising a V_{H} domain, a GS linker and a V_{L} domain. V_{H} CDR and V_{L} CDR sequences are marked in a lighter colored font. FIG. 10D shows the flow cytometric analysis showing expression levels of Chimeric antigen receptor (CAR) and low-affinity nerve growth factor receptor (LNGFR) for human m.uPAR-h.28z and h.19-h.28z human CAR T cells. Representative results of four independent experiments are shown. FIGS. 10E-10H show nonlimiting examples of anti-human uPAR scFv of the present technology. FIG. 10E shows the nucleotide sequence (SEQ ID NO: 56) of an anti-human uPAR scFv comprising a V_{H} domain, a GS linker and a V_{L} domain (construct 1). FIG. 10F shows the amino acid sequence (SEQ ID NO: 53) of an anti-human uPAR scFv comprising a V_{H} domain, a GS linker and a V_{L} domain (construct 1). FIG. 10G shows the nucleotide sequence (SEQ ID NO: 57) of an anti-human uPAR scFv comprising a V_{H} domain, a GS linker and a V_{L} domain (construct 2). FIG. 10H shows the amino acid sequence (SEQ ID NO: 54) of an anti-human uPAR scFv comprising a V_{H} domain, a GS linker and a V_{L} domain (construct 2).

FIG. 11A shows the flow cytometric analysis of murine uPAR (m.uPAR) and human CD19 (h.CD19) on wild type (WT) NALM6 cells and on NALM6 cells genetically engineered to overexpress murine uPAR (NALM6-m.uPAR). Representative results of three independent experiments are shown. FIG. 11B shows the cytotoxic activity as determined by an 18 hr-bioluminescence assay with FFLuc-expressing NALM6 WT or NALM6-m.uPAR as targets. Representative results of three independent experiments are shown. FIG. 11C shows the cytotoxic activity of m.uPAR-h.28z, h.19-h.28z and untransduced (UT) T cells as determined by 4 hr-Calcein assay with FFLuc-expressing wild-type (WT) NALM6 or NALM6-m.uPAR as targets. Representative results of three independent experiments are shown. FIG. 11D shows the cytotoxic activity as determined by a 4 hr-bioluminescence assay with murine KP cells induced to senescence by treatment with MEK and CDK4/6 inhibitors (MEKi, CDK4/6i) as targets. Representative results of three independent experiments are shown. FIG. 11E shows the granzyme B (GrB) and interferon γ (IFNγ) expression on CD4+ and CD8+m.uPAR-h.28z or h.19-h.28z CAR T cells 18 hours after co-culture with NALM6 WT, NALM6-m.uPAR or senescent KP cells as determined by intracellular cytokine staining. Results of one independent experiment are shown. FIG. 11F shows the expression of activation and exhaustion markers on m.uPAR-h.28z and h.CD19-h.28z CAR T cells as compared to untransduced T cells (UT) after coculture with NALM6-m.uPAR cells for 24 hr. Results of one independent experiment are shown. FIG. 11G shows the phenotype of m.uPAR-h.28z and h.CD19-h.28z CAR T cells without (left) and after (right) coculture with NALM6-m.uPAR cells for 24 hr as determined by flow cytometric expression of CD62L/CD45RA. Results of one independent experiment are shown. FIG. 11H shows the expression of mouse uPAR (m.uPAR) on the surface of mouse m.uPAR-m.28z, m.CD19-m.28z and UT T cells as compared to FMO control.

FIG. 12A shows the flow cytometric analysis showing expression levels of Myc-tag for murine m.uPAR-m.28z and m.19-m.28z CAR T cells as compared to untransduced (UT) controls. Representative results of three independent experiments are shown. FIG. 12B shows the flow cytometric analysis of murine uPAR (m.uPAR) and murine CD19 (m.CD19) expression on wild type (WT) Eμ-ALL01 cells and on Eμ-ALL01 cells engineered to overexpress murine uPAR (Eμ-ALL01-m.uPAR). Representative results of three independent experiments are shown. FIG. 12C shows the cytotoxic activity as determined by an 18 hr-bioluminescence assay with FFLuc-expressing Eμ-ALL01 WT or Eμ-ALL01-m.uPAR as targets. Representative results of two independent experiments are shown. FIG. 12D shows the cytotoxic activity as determined by an 18 hr-bioluminescence assay using murine KP cells as targets, which were induced to senescence by treatment with a MEKi and a CDK4/6i. Results of one independent experiment are shown.

FIGS. 13A-13E demonstrate that anti-uPAR CAR-T cells selectively target uPAR positive cells in vivo. FIG. 13A shows the experimental scheme used to assay in vivo cytotoxicity of anti-uPAR CAR T cells. NSG mice were injected with 0.5×10⁶ NALM6-uPAR cells on day 0. On day 5, mice received either no treatment, untransduced T cells (UT) or CD19-28z-CAR T cells (CD19 CAR T) or uPAR-28z-CAR T cells (uPAR CAR T). FIG. 13B shows tumor measurements as indicated by luciferase signal at day 12 post NALM6-uPAR injection (7 days after CAR T injection). FIG. 13C shows the tumor growth in the different cohorts (each line represents a different mouse). FIG. 13D shows the number of CAR T cells, the number of NALM6 tumor cells and the ratio CAR T cells/NALM6 tumor cells in the bone marrow at day 15 as measured by flow cytometry. FIG. 13E shows a Kaplan-Meier survival curve for the different treatment groups.

FIGS. 14A-14H demonstrate that uPAR CAR T cells are bona fide in vivo senolytics. FIG. 14A shows the experimental layout: NSG mice were injected with a plasmid encoding NRAS^{G12D}-GFP-Luciferase and intravenously (i.v.) treated with 0.5×10⁶ human m.uPAR-h.28z CAR T cells or untransduced (UT) T cells 10 days after injection. Mice were euthanized 15 days after CAR administration and livers were used for further immunohistological and flow cytometric analyses. FIG. 14B shows the n fold increase in luciferase signal in mice (calculated as average radiance on day x post CAR infusion divided by average radiance on day −1 prior to CAR injection) and representative bioluminescence images of the mice at day 15 post CAR infusion (n=5 mice per group). FIG. 14C (left panel) shows the co-immunofluorescence staining of murine uPAR (red) and NRAS (green) in the livers of mice treated with m.uPAR-h.28z or UT T cells and quantification of NRAS-positive cells in the livers of respective mice (n=4 mice per group). FIG. 14C (right panel) shows the number of NRAS+ cells in the images shown in FIG. 14C (left panel).

FIG. 14D shows the representative co-immunofluorescence staining of murine uPAR (red) and human CD3 (green) in the livers of mice treated with m.uPAR-h.28z CARs as compared to untransduced (UT) T cells. FIG. 14E (left panel) shows the representative SA-β-Gal stainings of the livers of treated mice and quantification of SA-β-Gal positive cells (n=3 mice per group). FIG. 14E (right panel) shows the percentage of SA-β-Gal expressing cells in the images shown in FIG. 14E (left panel). FIG. 14F shows the number of CAR T cells in the livers of mice receiving m.uPAR-h.28z vs. UT T cells as determined by flow cytometric analysis (n=4 mice per group). FIG. 14G shows the expression of CD62L and CD45RA on m.uPAR-h.28z CAR T cells present in the livers at day 15 post CAR injection (n=4 mice per group). FIG. 14H shows the percentage of PD1+TIM3+LAG3+ expression on m.uPAR-h.28z CAR T cells in the livers of treated mice (n=4 mice per group). In FIGS. 14B, 14C, and 14E, results are representative of three independent experiments (n=5 mice per group). All data are mean±SEM. Two-tailed unpaired Student's t-test. *P<0.05, **P<0.01, ***P<0.001.

FIG. 15A shows an experimental scheme for assessing SASP modulation of CAR T cell activation. FIG. 15B shows the fold change in the surface expression of activation markers in uPAR-28z-CAR T cells cultured for 24 h with either: DMEM alone (−), PMA and ionomycin (+), or supernatant from proliferative or senescent fibroblasts.

FIGS. 16A-16F demonstrate that senolytic CAR T cells show therapeutic efficacy in liver fibrosis. FIG. 16A shows the experimental layout: C57BL/6J mice received semiweekly intraperitoneal (i.p.) infusions of CCl₄ for 6 weeks and were intravenously (i.v.) infused with 3×10⁶ murine m.uPAR-m.28z murine CAR T cells 24 hr after cyclophosphamide (200 mg/kg) administration. Mice were euthanized 20 days after CAR infusion to assess liver fibrosis. FIG. 16B shows the representative levels of fibrosis evaluated by Sirius red staining and SA-β-Gal staining of livers from mice treated with m.uPAR-m.28z compared to UT T cells and quantification of liver fibrosis and SA-β-Gal+ cells in the livers (n=3 mice per group). FIG. 16C shows the co-immunofluorescence staining of either murine uPAR (red) and smooth muscle actin (green) or Myc-tag (red) and smooth muscle actin (green) in the livers of treated mice. FIG. 16D shows the fold change difference in plasma levels of suPAR 20 days after CAR T cell treatment as compared to day −1 before CAR T cell injection (n=5 mice per group). FIGS. 16E-16F show the levels of serum alanine transaminase (ALT) (U/L) (FIG. 16E) and albumin (g/dl) (FIG. 16F) in mice treated with m.uPAR-m.28z CARs or UT T cells 20 days after CAR treatment (n=5 mice per group). In FIGS. 16B, 16D, 16E, and 16F, results of one independent experiment (n=5 mice per group) are shown. All data represent mean±SEM. Two-tailed unpaired Student's t-test. *P<0.05, ***P<0.001.

FIGS. 17A-17F demonstrate that m.uPAR-h.28z CAR T cells show therapeutic efficacy in liver fibrosis. FIG. 17A shows the experimental layout: NSG mice were intraperitoneally (i.p.) injected with CCl₄ semiweekly for 6 weeks, followed by an infusion of 0.5×10⁶ human m.uPAR-h.28z CARs or untransduced (UT) T cells. CCl₄ injections were continued once per week after CAR infusion. FIG. 17B shows the fold change in the plasma levels of suPAR obtained from mice treated with m-uPAR-h.28z CAR or UT T cells at day 13 post CAR T cell injection as compared to day −1 (n=5 mice per group). FIGS. 17C-17D show the levels of serum alanine transaminase (ALT) (FIG. 17C) and levels of albumin (FIG. 17D) in the plasma of mice at day 13 post CAR injection (n=5 mice per group). FIG. 17E shows the representative Sirius red and SA-β-Gal stainings in liver sections of treated mice 13 days post CAR injection and quantification of liver fibrosis and SA-β-Gal+ cells in the livers (n=3 mice per group). FIG. 17F shows the co-immunofluorescence stainings for murine uPAR (m.uPAR, red) and smooth muscle actin (green) or m.uPAR (red) and human CD3 (green) in liver sections 13 days post CAR injection. In FIGS. 17B, 17C, 17D, and 17E, results are representative of two independent experiments (n=4-6 mice per group). Two-tailed unpaired Student's t-test. Data represent mean±SEM. *P<0.05, **P<0.01, ***P<0.001.

FIGS. 18A-18C: Senolytic CAR T cells show therapeutic efficacy in a model of liver fibrosis induced by Non-Alcoholic SteatoHepatitis (NASH). FIG. 18A: Experimental layout: C57Bl/6J mice were placed on continuous NASH diet for three months and were then intravenously (iv) infused with 0.5×10⁶ m.uPAR-m.28z murine CAR T cells 16 hr after cyclophosphamide (200 mg/kg) administration. Mice were kept on NASH diet and euthanized 20 days after CAR infusion to assess liver fibrosis. FIG. 18B: Representative levels of fibrosis evaluated by Sirius Red staining and SA-B-Gal stainings of livers from mice treated with m.uPAR-m.28z murine CAR T cells compared to untransduced (UT) T cells. Quantifications of liver fibrosis and SA-B-Gal positive cells in the livers shown at the right. (n=4-5 mice per group). FIG. 18C: Levels of albumin (g/dl) in mice treated with m.uPAR-m.28z CARs or UT T cells 20 days after CAR treatment. (n=4-5 mice per group). All data represent mean±SEM. Two-tailed unpaired Student's t-test.

FIGS. 19A-19B: Senolytic CAR T cells allow for a one-two punch senogenic-senolytic therapeutic approach in lung cancer. FIG. 19A: Experimental layout: C57Bl/6J mice were intravenously injected with X murine Kras^{G12D};p53^{-/-} cells (KP cells). 7 days after injection the animals started treatment with a Cdk4/6 inhibitor (100 mg/kg) and a MEK inhibitor (1 mg/kg). 7 days after treatment initiation (14 days since injection), mice were intravenously injected with either 2×10⁶ m.uPAR-m.28z murine CAR T cells, 2×10⁶ m.19-m.28z murine CAR T cells or 2×10⁶ untransduced T cells 24 hr after cyclophosphamide (200 mg/kg) administration. FIG. 19B: Kaplan-Meier survival curve of KP transplant mice treated with combined Cdk4/6 inhibitor (100 mg/kg) and a MEK inhibitor (1 mg/kg) and either 2×10⁶ m.uPAR-m.28z murine CAR T cells, 2×10⁶ m.19-m.28z murine CAR T cells or 2×10⁶ untransduced T cells. (n=7-9 mice per group). Log-rank test.

FIGS. 20A-20C show gating strategies. FIGS. 20A-20B show the representative flow cytometric staining of m.uPAR-h.28z CAR T cells (FIG. 20A) or untransduced T cells (FIG. 20B) obtained from the livers of mice that had undergone hydrodynamic tail vein injections (HTVI) (as depicted in FIG. 14). FIG. 20C shows an illustration summarizing key points of the results disclosed herein. uPAR-28z CAR T cells (depicted in red and marked by black arrowheads) infiltrate fibrotic livers containing senescent cells (depicted in blue and marked by grey arrowheads) and efficiently eliminate them leading to fibrosis resolution and improved liver function.

FIGS. 21A-21C show activity of anti-human uPAR CAR T cells. FIG. 21A shows a construct map encoding human h.uPAR-h.28z CAR T cells. The amino acid sequences of huPAR28z-LNGFR Nr. 1 and huPAR28z-LNGFR Nr. 2 are shown in FIG. 10F and FIG. 10H, respectively. FIG. 21B depicts flow cytometric analysis showing expression levels of CAR and LNGFR for human h.uPAR-h.28z CAR T cells. FIG. 21C shows cytotoxic activity as determined by an 18 hr-bioluminiscence assay with FFL-expressing NALM6 human uPAR as targets.

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

The present disclosure demonstrates that the uPAR surface protein is commonly upregulated in a broad range of in vitro and in vivo mammalian models for senescence. As demonstrated in the Examples herein, upregulation of uPAR occurred in response to all tested senescence triggers: replication induced senescence, drug induced senescence (e.g., combined MEK and CDK4/6 inhibition or Doxorubicin), and oncogene induced senescence (oncogenic Ras). Indeed, soluble uPAR (suPAR) plasma levels were positively correlated with the load of senescent cells present in the organism. Furthermore, the engineered immune cells of the present technology selectively targeted senescent cells, while leaving normal proliferating cells unaffected. In particular, the engineered immune cells disclosed herein efficiently eliminated lymphoma cells that ectopically expressed uPAR cDNA, without causing any unwanted/toxic side effects to the host animals. Accordingly, the selective senolytic engineered immune cells of the present technology are useful in methods for treating or ameliorating the effects of senescence-associated pathologies, such as lung fibrosis, atherosclerosis, Alzheimer's disease, diabetes, liver fibrosis, chronic kidney disease, aging, or osteoarthritis, and improving tumor responsiveness in subjects receiving senescence-inducing therapies (e.g., chemotherapeutic agents).

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the singular forms “a”, “an” and “the” are intended to include the plural forms as well, unless the context clearly indicates otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value.

As used herein, the term “administration” of an agent to a subject includes any route of introducing or delivering the agent to a subject to perform its intended function. Administration can be carried out by any suitable route, including, but not limited to, intravenously, intramuscularly, intraperitoneally, subcutaneously, and other suitable routes as described herein. Administration includes self-administration and the administration by another.

As used herein “adoptive cell therapeutic composition” refers to any composition comprising cells suitable for adoptive cell transfer. In exemplary embodiments, the adoptive cell therapeutic composition comprises a cell type selected from the group consisting of a tumor infiltrating lymphocyte (TIL), TCR (i.e., heterologous T-cell receptor), modified lymphocytes, and CAR (i.e., chimeric antigen receptor) modified lymphocytes. In another embodiment, the adoptive cell therapeutic composition comprises a cell type selected from the group consisting of T-cells, CD8⁺ cells, CD4⁺ cells, NK-cells, delta-gamma T-cells, regulatory T-cells and peripheral blood mononuclear cells. In another embodiment, TILs, T-cells, CD8⁺ cells, CD4⁺ cells, NK-cells, delta-gamma T-cells, regulatory T-cells or peripheral blood mononuclear cells form the adoptive cell therapeutic composition. In one embodiment, the adoptive cell therapeutic composition comprises T cells.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form, and a second plurality of amino acids are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, the term “analog” refers to a structurally related polypeptide or nucleic acid molecule having the function of a reference polypeptide or nucleic acid molecule.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab. F(ab′)2, and Fab fragments that lack the Fc fragment of intact antibody, clear more rapidly from the circulation, and may have less non-specific tissue binding of an intact antibody (Wahl et al., J. Nucl. Med. 24:316-325 (1983)). Antibodies may comprise whole native antibodies, monoclonal antibodies, human antibodies, humanized antibodies, camelised antibodies, multispecific antibodies, bispecific antibodies, chimeric antibodies, Fab, Fab′, single chain V region fragments (scFv), single domain antibodies (e.g., nanobodies and single domain camelid antibodies), VNAR fragments, Bi-specific T-cell engager (BiTE) antibodies, minibodies, disulfide-linked Fvs (sdFv), and anti-idiotypic (anti-Id) antibodies, intrabodies, fusion polypeptides, unconventional antibodies and antigen binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA, and IgY), class (e.g., IgG1, IgG2, IgG3, IgG4, IgA1, and IgA2), or subclass.

In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, C_{H}1, C_{H}2, and C_{H}3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Cl q) of the classical complement system. As used herein interchangeably, the terms “antigen binding portion”, “antigen binding fragment”, or “antigen binding region” of an antibody, refer to the region or portion of an antibody that binds to the antigen and which confers antigen specificity to the antibody; fragments of antigen binding proteins, for example antibodies, include one or more fragments of an antibody that retain the ability to specifically bind to an antigen (e.g., an peptide/HLA complex). It has been shown that the antigen binding function of an antibody can be performed by fragments of a full-length antibody. Examples of antigen binding portions encompassed within the term “antibody fragments” of an antibody include a Fab fragment, a monovalent fragment consisting of the V_{L}, V_{H}, C_{L} and C_{H}1 domains; a F(ab)₂ fragment, a bivalent fragment comprising two Fab fragments linked by a disulfide bridge at the hinge region; a Fd fragment consisting of the V_{H} and C_{H}1 domains; a Fv fragment consisting of the V_{L} and V_{H} domains of a single arm of an antibody; a dAb fragment (Ward et al., Nature 341: 544-546 (1989)), which consists of a V_{H} domain; and an isolated complementarity determining region (CDR). An “isolated antibody” or “isolated antigen binding protein” is one which has been identified and separated and/or recovered from a component of its natural environment. “Synthetic antibodies” or “recombinant antibodies” are generally generated using recombinant technology or using peptide synthetic techniques known to those of skill in the art.

Antibodies and antibody fragments can be wholly or partially derived from mammals (e.g., humans, non-human primates, goats, guinea pigs, hamsters, horses, mice, rats, rabbits and sheep) or non-mammalian antibody producing animals (e.g., chickens, ducks, geese, snakes, and urodele amphibians). The antibodies and antibody fragments can be produced in animals or produced outside of animals, such as from yeast or phage (e.g., as a single antibody or antibody fragment or as part of an antibody library).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science 242:423-426 (1988); and Huston et al., Proc. Natl. Acad. Sci. 85: 5879-5883 (1988). These antibody fragments are obtained using conventional techniques known to those of ordinary skill in the art, and the fragments are screened for utility in the same manner as are intact antibodies.

As used herein, an “antigen” refers to a molecule to which an antibody can selectively bind. The target antigen may be a protein (e.g., an antigenic peptide), carbohydrate, nucleic acid, lipid, hapten, or other naturally occurring or synthetic compound. An antigen may also be administered to an animal subject to generate an immune response in the subject.

By “binding affinity” is meant the strength of the total noncovalent interactions between a single binding site of a molecule (e.g., an antibody) and its binding partner (e.g., an antigen). Without wishing to be bound by theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes (e.g., either monovalent or multivalent). Methods for calculating the affinity of an antibody for an antigen are known in the art, comprising use of binding experiments to calculate affinity. The affinity of a molecule X for its partner Y can generally be represented by the dissociation constant (K_{d}). A low-affinity complex contains an antibody that generally tends to dissociate readily from the antigen, whereas a high-affinity complex contains an antibody that generally tends to remain bound to the antigen for a longer duration. Antibody activity in functional assays (e.g., flow cytometry assay) is also reflective of antibody affinity. Antibodies and affinities can be phenotypically characterized and compared using functional assays (e.g., flow cytometry assay).

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. Sequences of Proteins of Immunological Interest, Fifth Edition, U.S. Department of Health and Human Services, NIH Publication No. 91-3242(1991)).

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

As used herein, the term “chimeric co-stimulatory receptor” or “CCR” refers to a chimeric receptor that binds to an antigen and provides co-stimulatory signals, but does not provide a T-cell activation signal.

As used herein, the term “conservative sequence modification” refers to an amino acid modification that does not significantly affect or alter the binding characteristics of the presently disclosed CAR (e.g., the extracellular antigen binding domain of the CAR) comprising the amino acid sequence. Conservative modifications can include amino acid substitutions, additions, and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine; negatively-charged amino acids include aspartic acid and glutamic acid; and neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term, “co-stimulatory signaling domain,” or “co-stimulatory domain”, refers to the portion of the CAR comprising the intracellular domain of a co-stimulatory molecule. Co-stimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD28 and 4-1BB, other costimulatory domains are contemplated for use with the CARs described herein. The inclusion of one or more co-stimulatory signaling domains can enhance the efficacy and expansion of T cells expressing CAR receptors. The intracellular signaling and co-stimulatory signaling domains can be linked in any order in tandem to the carboxyl terminus of the transmembrane domain.

As used herein, the term “disease” refers to any condition or disorder that damages or interferes with the normal function of a cell, tissue, or organ.

As used herein, the term “effective amount” or “therapeutically effective amount” refers to a quantity of an agent sufficient to achieve a beneficial or desired clinical result upon treatment. In the context of therapeutic applications, the amount of a therapeutic agent administered to the subject can depend on the type and severity of the disease or condition and on the characteristics of the individual, such as general health, age, sex, body weight, effective concentration of the engineered immune cells administered, and tolerance to drugs. It can also depend on the degree, severity, and type of disease. The skilled artisan will be able to determine appropriate dosages depending on these and other factors. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay and Bradford protein assay.

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

As used herein, the term “heterologous nucleic acid molecule or polypeptide” refers to a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

As used herein, a “host cell” is a cell that is used to receive, maintain, reproduce and amplify a vector. A host cell also can be used to express the polypeptide encoded by the vector. The nucleic acid contained in the vector is replicated when the host cell divides, thereby amplifying the nucleic acids.

As used herein, the term “immune cell” refers to any cell that plays a role in the immune response of a subject. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes. As used herein, the term “engineered immune cell” refers to an immune cell that is genetically modified. As used herein, the term “native immune cell” refers to an immune cell that naturally occurs in the immune system.

As used herein, the term “immunoresponsive cell” refers to a cell that functions in an immune response or a progenitor, or progeny thereof.

As used herein, the term “increase” means to alter positively by at least about 5%, including, but not limited to, alter positively by about 5%, by about 10%, by about 25%, by about 30%, by about 50%, by about 75%, or by about 100%.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “ligand” refers to a molecule that binds to a receptor. In particular, the ligand binds a receptor on another cell, allowing for cell-to-cell recognition and/or interaction.

The term “linker” refers to synthetic sequences (e.g., amino acid sequences) that connect or link two sequences, e.g., that link two polypeptide domains. In some embodiments, the linker contains 1, 2, 3, 4, 5, 6, 7, 8, 9, or 10 amino acid residues.

The term “lymphocyte” refers to all immature, mature, undifferentiated, and differentiated white blood cell populations that are derived from lymphoid progenitors including tissue specific and specialized varieties, and encompasses, by way of non-limiting example, B cells, T cells, NKT cells, and NK cells. In some embodiments, lymphocytes include all B cell lineages including pre-B cells, progenitor B cells, early pro-B cells, late pro-B cells, large pre-B cells, small pre-B cells, immature B cells, mature B cells, plasma B cells, memory B cells, B-1 cells, B-2 cells, and anergic AN1/T3 cell populations.

As used herein, the term “modulate” means to positively or negatively alter. Exemplary modulations include an about 1%, about 2%, about 5%, about 10%, about 25%, about 50%, about 75%, or about 100% change.

As used herein, “operably linked” with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide affects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, the “percent homology” between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4: 1 1-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

The terms “polypeptide,” “peptide,” and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full length proteins, wherein the amino acid residues are linked by covalent peptide bonds.

As used herein, the term “reduce” means to alter negatively by at least about 5% including, but not limited to, alter negatively by about 5%, by about 10%, by about 25%, by about 30%, by about 50%, by about 75%, or by about 100%.

As used herein, “regulatory region” of a nucleic acid molecule means a cis-acting nucleotide sequence that influences expression, positively or negatively, of an operably linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration, gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more. Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, the term “sample” refers to clinical samples obtained from a subject. In certain embodiments, a sample is obtained from a biological source (i.e., a “biological sample”), such as tissue, bodily fluid, or microorganisms collected from a subject. Sample sources include, but are not limited to, mucus, sputum, bronchial alveolar lavage (BAL), bronchial wash (BW), whole blood, bodily fluids, cerebrospinal fluid (CSF), urine, plasma, serum, or tissue.

As used herein, the term “secreted” in reference to a polypeptide means a polypeptide that is released from a cell via the secretory pathway through the endoplasmic reticulum, Golgi apparatus, and as a vesicle that transiently fuses at the cell plasma membrane, releasing the proteins outside of the cell. Small molecules, such as drugs, can also be secreted by diffusion through the membrane to the outside of cell.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen binding domain. In certain embodiments, the linker comprises amino acids having the sequence set forth in SEQ ID NO: 1 as provided below: GGGGSGGGGSGGGGS (SEQ ID NO: 1). In certain embodiments, the nucleic acid sequence encoding the amino acid sequence of SEQ ID NO: 1 is set forth in SEQ ID NO: 2, which is provided below: ggcggcggcggatctggaggtggtggctcaggtggcggaggctcc (SEQ ID NO: 2).

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 85:5879-5883 (1988)). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 27(6):455-51 (2008); Peter et al., J Cachexia Sarcopenia Muscle (2012); Shieh et al., J Imunol 183(4):2277-85 (2009); Giomarelli et al., Thromb Haemost 97(6):955-63 (2007); Fife et al., J Clin Invst 116(8):2252-61 (2006); Brocks et al., Immunotechnology 3(3): 173-84 (1997); Moosmayer et al., Ther Immunol 2(10):31-40 (1995). Agonistic scFvs having stimulatory activity have been described (see, e.g., Peter et al., J Biol Chem 25278(38):36740-7 (2003); Xie et al., Nat Biotech 15(8):768-71 (1997); Ledbetter et al., Crit Rev Immunol 17(5-6):427-55 (1997); Ho et al., Bio Chim Biophys Acta 1638(3):257-66 (2003)).

As used herein, the term “specifically binds” or “specifically binds to” or “specifically target” refers to a molecule (e.g., a polypeptide or fragment thereof) that recognizes and binds a molecule of interest (e.g., an antigen), but which does not substantially recognize and bind other molecules. The terms “specific binding,” “specifically binds to,” or is “specific for” a particular molecule (e.g., an antigen), as used herein, can be exhibited, for example, by a molecule having a K_{d} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M.

As used herein, the terms “subject,” “individual,” or “patient” are used interchangeably and refer to an individual organism, a vertebrate, or a mammal and may include humans, non-human primates, rodents, and the like (e.g., which is to be the recipient of a particular treatment, or from whom cells are harvested). In certain embodiments, the individual, patient or subject is a human.

The terms “substantially homologous” or “substantially identical” mean a polypeptide or nucleic acid molecule that exhibits at least 50% or greater homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or about 99% homologous or identical at the amino acid level or nucleic acid to the sequence used for comparison (e.g., a wild-type, or native, sequence). In some embodiments, a substantially homologous or substantially identical polypeptide contains one or more amino acid substitutions, insertions, or deletions relative to the sequence used for comparison. In some embodiments, a substantially homologous or substantially identical polypeptide contains one or more non-natural amino acids or amino acid analogs, including, D-amino acids and retroinverso amino, to replace homologous sequences.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e-3 and e-100 indicating a closely related sequence.

Nucleic acid molecules useful in the presently disclosed subject matter include any nucleic acid molecule that encodes a polypeptide or a fragment thereof. In certain embodiments, nucleic acid molecules useful in the presently disclosed subject matter include nucleic acid molecules that encode an antibody or an antigen binding portion thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, Methods Enzymol. 152:399 (1987); Kimmel, A. R. Methods Enzymol. 152:507 (1987)). For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% w/v formamide, or at least about 50% w/v formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% w/v SDS. In certain embodiments, hybridization will occur at 37° C. in 500 mM NaCl, 50 mM trisodium citrate, 1% w/v SDS, 35% w/v formamide, and 100 μg/ml denatured salmon sperm DNA (ssDNA). In certain embodiments, hybridization will occur at 42° C. in 250 mM NaCl, 25 mM trisodium citrate, 1% w/v SDS, 50% w/v formamide, and 200 μg ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., at least about 42° C., or at least about 68° C. In certain embodiments, wash steps will occur at 25° C. in 30 mM NaCl, 3 mM trisodium citrate, and 0.1% w/v SDS. In certain embodiments, wash steps will occur at 42° C. in 15 mM NaCl, 1.5 mM trisodium citrate, and 0.1% w/v SDS. In certain embodiments, wash steps will occur at 68° C. in 15 mM NaCl, 1.5 mM trisodium citrate, and 0.1% w/v SDS. Additional variations on these conditions will be readily apparent to those skilled in the art. Hybridization techniques are well known to those skilled in the art and are described, for example, in Benton and Davis (Science 196: 180 (1977)); Grunstein and Rogness (Proc. Natl. Acad. Sci., USA 72:3961 (1975)); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods. As used herein, “production by recombinant means by using recombinant DNA methods” means the use of the well-known methods of molecular biology for expressing proteins encoded by cloned DNA.

As used herein, the term “T-cell” includes naïve T cells, CD4⁺ T cells, CD8⁺ T cells, memory T cells, activated T cells, anergic T cells, tolerant T cells, chimeric B cells, and antigen-specific T cells.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. Therapeutic effects of treatment include, without limitation, inhibiting recurrence of disease, alleviation of symptoms, diminishment of any direct or indirect pathological consequences of the disease, preventing metastases, decreasing the rate of disease progression, amelioration or palliation of the disease state, and remission or improved prognosis. By “treating a cancer” is meant that the symptoms associated with the cancer are, e.g., alleviated, reduced, cured, or placed in a state of remission.

It is also to be appreciated that the various modes of treatment of diseases as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

As used herein “tumor-infiltrating lymphocytes” or “TILs” refer to white blood cells that have left the bloodstream and migrated into a tumor.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art. A vector also includes “virus vectors” or “viral vectors.” Viral vectors are engineered viruses that are operably linked to exogenous genes to transfer (as vehicles or shuttles) the exogenous genes into cells. As used herein, an “expression vector” includes vectors capable of expressing DNA that is operably linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

### CAR T Cell Therapy Overview

CAR T cell therapy has gained momentum after several promising clinical trials for the treatment of B-cell neoplasms and the FDA approval of a CD19 targeted CAR T cell for treatment of B cell acute lymphoid leukemia (Sadelain et al., Nature 545:423-431 (2017); Yu et al., J Hematol Oncol. 10:78 (2017); Kakarla and Gottschalk, Cancer J. 20:151-155 (2014); Wang et al., J Hematol Oncol. 10:53 (2017)). CAR T cell therapy involves isolating a patient's own T cells, engineering them to express a CAR, and reinfusing the engineered T cells back into the patient. The CAR contains an extracellular single-chain variable fragment (scFv), a transmembrane domain, and an intracellular signaling domain. Surface expression of a tumor-targeted scFv on the T cell results in tumor antigen-directed T cell activation and specific tumor killing via its signaling domain. However, many patients with hematologic cancers treated with CAR T cell therapy relapse with antigen loss variants as a result of tumor editing (Wang et al., J Hematol Oncol. 10:53 (2017)). Furthermore, translation of CAR T cell therapy to solid tumors has been difficult due to the immunosuppressive tumor environment (TME) (Yu et al., J Hematol Oncol. 10:78 (2017); Kakarla and Gottschalk, Cancer J. 20:151-155 (2014)).

The TME consists of physical barriers, such as surrounding fibroblasts and extracellular matrix proteins, which make tumors less accessible to the T cells. Beyond this dense stromal network, T cell can encounter a number of inhibitory immune cells such as regulatory T cells, myeloid suppressor cells and tumor associated macrophages, as well an upregulation of immune checkpoint molecules, rendering the cytotoxic T cells inactive (Newick et al., Annu Rev Med. 1-14 (2016)). These immune checkpoints normally play a role in self recognition to prevent autoimmune responses, but are upregulated by many cancers to suppress immune cells (Topalian et al., Cancer Cell 27:451-461 (2015) and Postow et al., J Clin Oncol. 33:1974-1982 (2015)).

When the immune checkpoint proteins CTLA-4 and PD-1 receptors are expressed on the T cell surface, they function through distinct mechanisms to downregulate T cell activity to prevent autoimmunity and maintain immunological homeostasis (Postow et al., J Clin Oncol. 33:1974-1982 (2015)). Although immune checkpoint blockade therapies have been successful in treating patients with various cancers, patient response rate is variable (Matlung et al., Immunol Rev. 276:145-164 (2017); Rizvi et al., Science 348:124-128 (2015); Chao et al., Cell 24:225-232 (2011)).

Another immune checkpoint pathway is the Cluster of Differentiation 47 (CD47)-Signal Regulatory Protein α (SIRPα) pathway. SIRPα is a transmembrane glycoprotein found predominately on myeloid cells, including macrophages, monocytes and dendritic cells. The extracellular domain consists of three IgG superfamily domains, including an N-terminal CD47-binding domain, and is associated with two immunoreceptor tyrosine-based inhibitory motifs (ITIMs), which serve as docking sites for tyrosine phosphatases (Matlung et al., Immunol Rev. 276:145-164 (2017); Chao et al., Cell 24:225-232 (2011); Brown and Frazier, Trends Cell Biol. 11:130-135 (2001)). CD47 is expressed ubiquitously at low levels as a self-recognition signal (Matlung et al., Immunol Rev. 276:145-164 (2017)). CD47 binding to SIRPα on macrophages causes ITIM activation, resulting in induction of the docked tyrosine phosphatase, Src homology region 2 domain containing phosphatase-1 (SHP-1). SHP-1 then initiates a dephosphorylation cascade, causing dephosphorylation of myosin at the phagocytic synapse, preventing phagocytosis.

Many cancers exploit this mechanism and upregulate CD47 to send this “do not eat me” signal to macrophages (Matlung et al., Immunol Rev. 276:145-164 (2017); Chao et al., Cell 24:225-232 (2011)). CV1, a peptide antagonist of the CD47-SIRPα pathway, potently synergizes with a multitude of mAbs, leading to decreased tumor burden and, in some cases, remissions in mice (Weiskopf et al., Science 341:1-13 (2014); Mathias et al., Leukemia 31(10):2254-2257 (2017)). CV1 is a truncated SIRPα variant with point mutations that increase its affinity for CD47, such that it outcompetes endogenous SIRPα (Weiskopf et al., Science 341:1-13 (2014)). Although CV1 has yet to enter human trials, other anti-CD47 agents in trials have shown toxicities including anemia, due to the ubiquitous expression of CD47 (Matlung et al., Immunol Rev. 276:145-164 (2017); Weiskopf et al., Science 341:1-13 (2014); Liu et al., PLoS One 10:1-23 (2015)). Thus there is a need for new methods to increase the efficacy of CAR T cell therapy in solid tumors, prevent antigen loss relapse in hematologic tumors, and to reduce potential toxicities relating to CD47 blockade. Provided herein are engineered immune cells, including compositions comprising engineered immune cells and methods of use thereof, that address these issues.

Target uPAR Antigen

The engineered immune cells provided herein express a T-cell receptor (TCR) or other cell-surface ligand that binds to a target antigen, such as a uPAR antigen. The cell-surface ligand can be any molecule that directs an immune cell to a target site (e.g., a tumor site). Exemplary cell surface ligands include, for example engineered receptors, or other specific ligands to achieve targeting of the immune cell to a target site. In some embodiments, the receptor is a T cell receptor. In some embodiments, the receptor, e.g., a T cell receptor, is non-native receptor (e.g., not endogenous to the immune cells). In some embodiments, the receptor is a chimeric antigen receptor (CAR), for example, a T cell CAR, that binds to a target antigen (uPAR).

In some embodiments, the target uPAR antigen expressed by a tumor cell. In some embodiments, the target uPAR antigen is expressed on the surface of a tumor cell. In some embodiments, the target uPAR antigen is a cell surface receptor. In some embodiments, the target uPAR antigen is a cell surface glycoprotein. In some embodiments, the target uPAR antigen is secreted by a tumor cell. In some embodiments, the target uPAR antigen is localized to the tumor microenvironment. In some embodiments, the target uPAR antigen is localized to the extracellular matrix or stroma of the tumor microenvironment. In some embodiments, the target uPAR antigen is expressed by one or more cells located within the extracellular matrix or stroma of the tumor microenvironment.

Without limiting the foregoing, exemplary cancers can be treated by targeting a uPAR antigen include breast cancer, endometrial cancer, ovarian cancer, colon cancer, lung cancer, stomach cancer, prostate cancer, renal cancer, pancreatic cancer, acute lymphoblastic leukemia (ALL), chronic lymphocytic leukemia (CLL), and acute myeloid leukemia (AML). Other exemplary diseases or conditions that can be treated or ameliorated by targeting a uPAR antigen include lung fibrosis, atherosclerosis, Alzheimer's disease, diabetes, liver fibrosis, chronic kidney disease, aging, or osteoarthritis.

Typical therapeutic anti-cancer mAb, like those that bind to CD19, recognize cell surface proteins, which constitute only a tiny fraction of the cellular protein content. Most mutated or oncogenic tumor associated proteins are typically nuclear or cytoplasmic. In certain instances, these intracellular proteins can be degraded in the proteasome, processed and presented on the cell surface by MIIC class I molecules as T cell epitopes that are recognized by T cell receptors (TCRs). The development of mAb that mimic TCR function, “TCR mimic (TCRm)” or “TCR-like”; (i.e., that recognize peptide antigens of key intracellular proteins in the context of MHC on the cell surface) greatly extends the potential repertoire of tumor targets addressable by potent mAb. TCRm Fab, or scFv, and mouse IgG specific for the melanoma Ags, NY-ESO-1, hTERT, MART 1, gp100, and PR1, among others, have been developed. The antigen binding portions of such antibodies can be incorporated into the CARs provided herein. HLA-A2 is the most common HLA haplotype in the USA and EU (about 40% of the population) (Marsh, S., Parham, P., Barber, L., The HLA FactsBook. 1 ed. The HLA FactsBook. Vol. 1. 2000: Academic Press. 416). Therefore, potent TCRm mAb and native TCRs against tumor antigens presented in the context of HLA-A2 are useful in the treatment of a large populations.

Accordingly, in some embodiments, the target uPAR antigen is a tumor antigen presented in the context of an MHC molecule. In some embodiments, the MHC protein is a MHC class I protein. In some embodiments, the MHC Class I protein is an HLA-A, HLA-B, or HLA-C molecules. In some embodiments, target uPAR antigen is a tumor antigen presented in the context of an HLA-A2 molecule.

### Chimeric Antigen Receptors

In some embodiments, the engineered immune cells provided herein express at least one chimeric antigen receptor (CAR). CARs are engineered receptors, which graft or confer a specificity of interest onto an immune effector cell. For example, CARs can be used to graft the specificity of a monoclonal antibody onto an immune cell, such as a T cell. In some embodiments, transfer of the coding sequence of the CAR is facilitated by nucleic acid vector, such as a retroviral vector.

There are currently three generations of CARs. In some embodiments, the engineered immune cells provided herein express a “first generation” CAR. “First generation” CARs are typically composed of an extracellular antigen binding domain (e.g., a single-chain variable fragment (scFv)) fused to a transmembrane domain fused to cytoplasmic/intracellular domain of the T cell receptor (TCR) chain. “First generation” CARs typically have the intracellular domain from the CD3ζ chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation.

In some embodiments, the engineered immune cells provided herein express a “second generation” CAR. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (e.g., CD3ζ). Preclinical studies have indicated that “Second Generation” CARs can improve the antitumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL).

In some embodiments, the engineered immune cells provided herein express a “third generation” CAR. “Third generation” CARs comprise those that provide multiple co-stimulation (e.g., CD28 and 4-1BB) and activation (e.g., CD3ζ).

In accordance with the presently disclosed subject matter, the CARs of the engineered immune cells provided herein comprise an extracellular antigen-binding domain, a transmembrane domain and an intracellular domain.

Extracellular Antigen-Binding Domain of a CAR. In certain embodiments, the extracellular antigen-binding domain of a CAR specifically binds a uPAR antigen. In certain embodiments, the extracellular antigen-binding domain is derived from a monoclonal antibody (mAb) that binds to a uPAR antigen. In some embodiments, the extracellular antigen-binding domain comprises an scFv. In some embodiments, the extracellular antigen-binding domain comprises a Fab, which is optionally crosslinked. In some embodiments, the extracellular binding domain comprises a F(ab)₂. In some embodiments, any of the foregoing molecules are included in a fusion protein with a heterologous sequence to form the extracellular antigen-binding domain. In certain embodiments, the extracellular antigen-binding domain comprises a human scFv that binds specifically to a uPAR antigen. In certain embodiments, the scFv is identified by screening scFv phage library with a uPAR antigen-Fc fusion protein.

In certain embodiments, the extracellular antigen-binding domain of a presently disclosed CAR has a high binding specificity and high binding affinity to a uPAR antigen. For example, in some embodiments, the extracellular antigen-binding domain of the CAR (embodied, for example, in a human scFv or an analog thereof) binds to a particular uPAR antigen with a dissociation constant (K_{d}) of about 1×10⁻⁵ M or less. In certain embodiments, the K_{d} is about 5×10⁻⁶ M or less, about 1×10⁻⁶ M or less, about 5×10⁻⁷ M or less, about 1×10⁻⁷ M or less, about 5×10⁻⁸ M or less, about 1×10⁻⁸ M or less, about 5×10⁻⁹ or less, about 4×10⁻⁹ or less, about 3×10⁻⁹ or less, about 2×10⁻⁹ or less, or about 1×10⁻⁹ M or less. In certain non-limiting embodiments, the K_{d} is from about 3×10⁻⁹ M or less. In certain non-limiting embodiments, the K_{d} is from about 3×10⁻⁹ to about 2×10⁻⁷.

Binding of the extracellular antigen-binding domain (embodiment, for example, in an scFv or an analog thereof) of a presently disclosed uPAR-specific CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the uPAR-specific CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the scFv of a presently disclosed uPAR-specific CAR is labeled with GFP.

In some embodiments, the extracellular antigen-binding domain of the expressed CAR binds to a uPAR antigen that is expressed by a tumor cell. In some embodiments, the extracellular antigen-binding domain of the expressed CAR binds to a uPAR antigen that is expressed on the surface of a tumor cell. In some embodiments, the extracellular antigen-binding domain of the expressed CAR binds to a uPAR antigen that is expressed on the surface of a tumor cell in combination with an MHC protein. In some embodiments, the MHC protein is a MHC class I protein. In some embodiments, the MHC Class I protein is an HLA-A, HLA-B, or HLA-C molecules. In some embodiments, the extracellular antigen-binding domain of the expressed CAR binds to a uPAR antigen that is expressed on the surface of a tumor cell not in combination with an MHC protein.

In some embodiments, the extracellular antigen-binding domain of the expressed CAR binds to a uPAR antigen. In some embodiments, the extracellular antigen-binding domain of the expressed CAR binds to a uPAR antigen presented in the context of an MHC molecule. In some embodiments, the extracellular antigen-binding domain of the expressed CAR binds to a uPAR antigen presented in the context of an HLA-A2 molecule.

In certain embodiments, the extracellular antigen-binding domain (e.g., human scFv) comprises a heavy chain variable (V_{H}) region and a light chain variable (V_{L}) region, optionally linked with a linker sequence, for example a linker peptide (e.g., SEQ ID NO: 1), between the heavy chain variable (V_{H}) region and the light chain variable (V_{L}) region. In certain embodiments, the extracellular antigen-binding domain is a human scFv-Fc fusion protein or full length human IgG with V_{H} and V_{L} regions.

In certain non-limiting embodiments, an extracellular antigen-binding domain of the presently disclosed CAR can comprise a linker connecting the heavy chain variable (V_{H}) region and light chain variable (V_{L}) region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises amino acids having the sequence set forth in SEQ ID NO: 1. In certain embodiments, the nucleotide sequence encoding the amino acid sequence of SEQ ID NO: 1 is set forth in SEQ ID NO: 2.

Additionally or alternatively, in some embodiments, the extracellular antigen-binding domain can comprise a leader or a signal peptide sequence that directs the nascent protein into the endoplasmic reticulum. The signal peptide or leader can be essential if the CAR is to be glycosylated and anchored in the cell membrane. The signal sequence or leader sequence can be a peptide sequence (about 5, about 10, about 15, about 20, about 25, or about 30 amino acids long) present at the N-terminus of the newly synthesized proteins that direct their entry to the secretory pathway.

In certain embodiments, the signal peptide is covalently joined to the N-terminus of the extracellular antigen-binding domain. In certain embodiments, the signal peptide comprises a human CD8 signal polypeptide comprising amino acids having the sequence set forth in SEQ ID NO: 3 as provided below: MALPVTALLLPLALLLHAARP (SEQ ID NO: 3).

The nucleotide sequence encoding the amino acid sequence of SEQ ID NO: 3 is set forth in SEQ ID NO: 4, which is provided below:

(SEQ ID NO: 4)
ATGGCCCTGCCAGTAACGGCTCTGCTGCTGCCACTTGCTCTGCTCCTCC
ATGCAGCCAGGCCT.

In certain embodiments, the signal peptide comprises a human CD8 signal polypeptide comprising amino acids having the sequence set forth in SEQ ID NO: 5 as provided below:

(SEQ ID NO: 5)
MALPVTALLLPLALLLHA.

The nucleotide sequence encoding the amino acid sequence of SEQ ID NO: 5 is set forth in SEQ ID NO: 6, which is provided below:

(SEQ ID NO: 6)
ATGGCTCTCCCAGTGACTGCCCTACTGCTTCCCCTAGCGCTTCTCCTGC
ATGCA.

In certain embodiments, the signal peptide comprises a mouse CD8 signal polypeptide comprising amino acids having the sequence set forth in SEQ ID NO: 7 as provided below:

(SEQ ID NO: 7)
MASPLTRFLSLNLLLLGESII.

The nucleotide sequence encoding the amino acid sequence of SEQ ID NO: 7 is set forth in SEQ ID NO: 8, which is provided below:

(SEQ ID NO: 8)
ATGGCCAGCCCCCTGACCAGGTTCCTGAGCCTGAACCTGCTGCTGCTG
GGCGAGAGCATCATC.

In certain embodiments, the signal peptide comprises a mouse CD8 signal polypeptide comprising amino acids having the sequence set forth in SEQ ID NO: 9 as provided below:

(SEQ ID NO: 9)
MASPLTRFLSLNLLLLGE.

The nucleotide sequence encoding the amino acid sequence of SEQ ID NO: 9 is set forth in SEQ ID NO: 10, which is provided below:

(SEQ ID NO: 10)
ATGGCCAGCCCCCTGACCAGGTTCCTGAGCCTGAACCTGCTGCTGCTGG
GCGAG.

Transmembrane Domain of a CAR. In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (e.g., a transmembrane peptide not based on a protein associated with the immune response), or a combination thereof.

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a UniProtKB Reference No: P10747 or NCBI Reference No: NP006130 (SEQ ID NO: 11), or fragments thereof, and/or may optionally comprise up to one or up to two or up to three conservative amino acid substitutions. In certain embodiments, the CD28 polypeptide can have an amino acid sequence that is a consecutive portion of SEQ ID NO: 11 which is at least 20, or at least 30, or at least 40, or at least 50, and up to 220 amino acids in length. Additionally or alternatively, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 11. In certain embodiments, the CAR of the present disclosure comprises a transmembrane domain comprising a CD28 polypeptide, and optionally an intracellular domain comprising a co-stimulatory signaling region that comprises a CD28 polypeptide. In certain embodiments, the CD28 polypeptide comprised in the transmembrane domain and the intracellular domain has an amino acid sequence of amino acids 114 to 220 of SEQ ID NO: 11. In certain embodiments, the CD28 polypeptide comprised in the transmembrane domain has an amino acid sequence of amino acids 153 to 179 of SEQ ID NO: 11.

SEQ ID NO: 11 is provided below:

(SEQ ID NO: 11)
MLRLLLALNLFPSIQVTGNKILVKQSPMLVAYDNALSCKYSYNLFSREF
RASLHKGLDSAVEVCWYGNYSQQLQVYSKTGFNCDGKLGNESVTFYLQN
LYQTDIYFCKIEVMYPPPYLDNEKSNGTIIHVKGKHLCPSPLFPGPSKP
FWVLVWGGVLACYSLLVTVAFIIFWVRSKRSRLLHSDYMNMTPRRPGPT
RKHYQPYAPPRDFAAYRS

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide comprised in the transmembrane domain (and optionally the intracellular domain (e.g., the co-stimulatory signaling region)) of the presently disclosed CAR (e.g., amino acids 114 to 220 of SEQ ID NO: 11 or amino acids 153 to 179 of SEQ ID NO: 11) comprises at least a portion of the sequence set forth in SEQ ID NO: 12 as provided below.

(SEQ ID NO: 12)
attgaagttatgtatcctcctccttacctagacaatgagaagagcaatg
gaaccattatccatgtgaaagggaaacacctttgtccaagtcccctatt
tcccggaccttctaagcccttttgggtgctggtggtggttggtggagtc
ctggcttgctatagcttgctagtaacagtggcctttattattttctggg
tgaggagtaagaggagcaggctcctgcacagtgactacatgaacatgac
tccccgccgccccgggcccacccgcaagcattaccagccctatgcccca
ccacgcgacttcgcagcctatcgctcc

In certain embodiments, the transmembrane domain comprises a CD8 polypeptide. The CD8 polypeptide can have an amino acid sequence that is at least about 85%, about 90%, about 95%, about 96%, about 97%, about 98%, about 99% or about 100%) homologous to SEQ ID NO: 13 (homology herein may be determined using standard software such as BLAST or FASTA) as provided below, or fragments thereof, and/or may optionally comprise up to one or up to two or up to three conservative amino acid substitutions. In certain embodiments, the CD8 polypeptide can have an amino acid sequence that is a consecutive portion of SEQ ID NO: 13 which is at least 20, or at least 30, or at least 40, or at least 50, and up to 235 amino acids in length. Additionally or alternatively, in various embodiments, the CD8 polypeptide has an amino acid sequence of amino acids 1 to 235, 1 to 50, 50 to 100, 100 to 150, 150 to 200, or 200 to 235 of SEQ ID NO: 13.

(SEQ ID NO: 13)
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPWKSGDKPSLSARYV

In certain embodiments, the transmembrane domain comprises a CD8 polypeptide comprising amino acids having the sequence set forth in SEQ ID NO: 14 as provided below:

(SEQ ID NO: 14)
PTTTPAPRPPTPAPTIASQPLSLRPEACRPAAGGAVHTRGLDFACDIYI
WAPLAGTCGVLLLSLVITLYCN

In accordance with the presently disclosed subject matter, a “CD8 nucleic acid molecule” refers to a polynucleotide encoding a CD8 polypeptide. In certain embodiments, the CD8 nucleic acid molecule encoding the CD8 polypeptide comprised in the transmembrane domain of the presently disclosed CAR (SEQ ID NO: 14) comprises nucleic acids having the sequence set forth in SEQ ID NO: 15 as provided below.

(SEQ ID NO: 15)
CCCACCACGACGCCAGCGCCGCGACCACCAACCCCGGCGCCCACGATC
GCGTCGCAGCCCCTGTCCCTGCGCCCAGAGGCGTGCCGGCCAGCGGCGG
GGGGCGCAGTGCACACGAGGGGGCTGGACTTCGCCTGTGATATCTACAT
CTGGGCGCCCCTGGCCGGGACTTGTGGGGTCCTTCTCCTGTCACTGGTT
ATCACCCTTTACTGCAAC

In certain non-limiting embodiments, a CAR can also comprise a spacer region that links the extracellular antigen-binding domain to the transmembrane domain. The spacer region can be flexible enough to allow the antigen-binding domain to orient in different directions to facilitate antigen recognition while preserving the activating activity of the CAR. In certain non-limiting embodiments, the spacer region can be the hinge region from IgGl, the CH2CH3 region of immunoglobulin and portions of CD3, a portion of a CD28 polypeptide (e.g., SEQ ID NO: 11), a portion of a CD8 polypeptide (e.g., SEQ ID NO: 13), a variation of any of the foregoing which is at least about 80%, at least about 85%, at least about 90%, or at least about 95% homologous thereto, or a synthetic spacer sequence. In certain non-limiting embodiments, the spacer region may have a length between about 1-50 (e.g., 5-25, 10⁻³⁰, or 30-50) amino acids.

Intracellular Domain of a CAR. In certain non-limiting embodiments, an intracellular domain of the CAR can comprise a CD3ζ polypeptide, which can activate or stimulate a cell (e.g., a cell of the lymphoid lineage, e.g., a T cell). CD3ζ comprises 3 ITAMs, and transmits an activation signal to the cell (e.g., a cell of the lymphoid lineage, e.g., a T cell) after antigen is bound. The CD3ζ polypeptide can have an amino acid sequence that is at least about 85%, about 90%, about 95%, about 96%, about 97%, about 98%, about 99% or about 100% homologous to the sequence having a NCBI Reference No: NP_932170 (SEQ ID NO: 16), or fragments thereof, and/or may optionally comprise up to one or up to two or up to three conservative amino acid substitutions.

In certain embodiments, the CD3ζ polypeptide can have an amino acid sequence that is a consecutive portion of SEQ ID NO: 17 which is at least 20, or at least 30, or at least 40, or at least 50, and up to 164 amino acids in length. Additionally or alternatively, in various embodiments, the CD3ζ polypeptide has an amino acid sequence of amino acids 1 to 164, 1 to 50, 50 to 100, 100 to 150, or 150 to 164 of SEQ ID NO: 17. In certain embodiments, the CD3ζ polypeptide has an amino acid sequence of amino acids 52 to 164 of SEQ ID NO: 17.

SEQ ID NO: 17 is provided below:

(SEQ ID NO: 17)
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR

In certain embodiments, the CD3ζ polypeptide has the amino acid sequence set forth in SEQ ID NO: 18, which is provided below:

(SEQ ID NO: 18)
RVKFSRSAEPPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain embodiments, the CD3ζ polypeptide has the amino acid sequence set forth in SEQ ID NO: 19, which is provided below:

(SEQ ID NO: 19)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In accordance with the presently disclosed subject matter, a “CD3ζ nucleic acid molecule” refers to a polynucleotide encoding a CD3ζ polypeptide. In certain embodiments, the CD3ζ nucleic acid molecule encoding the CD3ζ polypeptide (SEQ ID NO: 18) comprised in the intracellular domain of the presently disclosed CAR comprises a nucleotide sequence as set forth in SEQ ID NO: 20 as provided below.

(SEQ ID NO: 20)
AGAGTGAAGTTCAGCAGGAGCGCAGAGCCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCG

In certain embodiments, the CD3ζ nucleic acid molecule encoding the CD3ζ polypeptide (SEQ ID NO: 19) comprised in the intracellular domain of the presently disclosed CAR comprises a nucleotide sequence as set forth in SEQ ID NO: 21 as provided below.

(SEQ ID NO: 21)
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCTAA

In certain non-limiting embodiments, an intracellular domain of the CAR further comprises at least one signaling region. The at least one signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the signaling region is a co-stimulatory signaling region.

In certain embodiments, the co-stimulatory signaling region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-Ll. As one example, a 4-1BB ligand (i.e., 4-1BBL) may bind to 4-1BB (also known as “CD 137”) for providing an intracellular signal that in combination with a CAR signal induces an effector cell function of the CAR⁺ T cell. CARs comprising an intracellular domain that comprises a co-stimulatory signaling region comprising 4-1BB, ICOS or DAP-10 are disclosed in U.S. Pat. No. 7,446,190, which is herein incorporated by reference in its entirety. In certain embodiments, the intracellular domain of the CAR comprises a co-stimulatory signaling region that comprises a CD28 polypeptide. In certain embodiments, the intracellular domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or 100% homologous to the sequence having a UniProtKB Reference No: P41273 or NCBI Reference No: NP_001552 (SEQ ID NO: 22) or fragments thereof, and/or may optionally comprise up to one or up to two or up to three conservative amino acid substitutions.

SEQ ID NO: 22 is provided below:

(SEQ ID NO: 22)
MGNSCYNIVATLLLVLNFERTRSLQDPCSNCPAGTFCDNNRNQICSPCP
PNSFSSAGGQRTCDICRQCKGVFRTRKECSSTSNAECDCTPGFHCLGAG
CSMCEQDCKQGQELTKKGCKDCCFGTFNDQKRGICRPWTNCSLDGKSVL
GTKERDWCGPSPADLSPGASSVTPPAPAREPGHSPQIISFFLALTSTAL
LFLLFFLTLRFSWKRGRKKLLYIFKQPFMRPVQTTQEEDGCSCRFPEEE
EGGCEL

In certain embodiments, the 4-1BB co-stimulatory domain has the amino acid sequence set forth in SEQ ID NO: 23, which is provided below:

(SEQ ID NO: 23)
KRGRKKLLYIFKQPFMRPVQTTQEEDGCSCRFPEEEEGGCEL

In accordance with the presently disclosed subject matter, a “4-1BB nucleic acid molecule” refers to a polynucleotide encoding a 4-1BB polypeptide. In certain embodiments, the 4-1BB nucleic acid molecule encoding the 4-1BB polypeptide (SEQ ID NO: 23) comprised in the intracellular domain of the presently disclosed CAR comprises a nucleotide sequence as set forth in SEQ ID NO: 24 as provided below.

(SEQ ID NO: 24)
AAACGGGGCAGAAAGAAGCTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTG

An OX40 polypeptide can have an amino acid sequence that is at least about 85%, about 90%, about 95%, about 96%, about 97%, about 98%, about 99% or 100% homologous to the sequence having a UniProtKB Reference No: P43489 or NCBI Reference No: NP_003318 (SEQ ID NO: 25), or fragments thereof, and/or may optionally comprise up to one or up to two or up to three conservative amino acid substitutions.

SEQ ID NO: 25 is provided below:

(SEQ ID NO: 25)
MCVGARRLGRGPCAALLLLGLGLSTVTGLHCVGDTYPSNDRCCHECRPG
NGMVSRCSRSQNTVCRPCGPGFYNDWSSKPCKPCTWCNLRSGSERKQLC
TATQDTVCRCRAGTQPLDSYKPGVDCAPCPPGHFSPGDNQACKPWTNCT
LAGKHTLQPASNSSDAICEDRDPPATQPQETQGPPARPITVQPTEAWPR
TSQGPSTRPVEVPGGRAVAAILGLGLVLGLLGPLAILLALYLLRRDQRL
PPDAHKPPGGGSFRTPIQEEQADAHSTLAKI

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO: 26) or fragments thereof, and/or may optionally comprise up to one or up to two or up to three conservative amino acid substitutions.

SEQ ID NO: 26 is provided below:

(SEQ ID NO: 26)
MKSGLWYFFLFCLRIKVLTGEINGSANYEMFIFHNGGVQILCKYPDIVQ
QFKMQLLKGGQILCDLTKTKGSGNTVSIKSLKFCHSQLSNNSVSFFLYN
LDHSHANYYFCNLSIFDPPPFKVTLTGGYLHIYESQLCCQLKFWLPIGC
AAFVWCILGCILICWLTKKKYSSSVHDPNGEYMFMRATAKKSRLTDVTL

In accordance with the presently disclosed subject matter, an “ICOS nucleic acid molecule” refers to a polynucleotide encoding an ICOS polypeptide.

CTLA-4 is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion, enhanced the endogenous anti-tumor response albeit, in the clinical setting, with significant unforeseen toxicities.

CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM motif (SEQ ID NO: 63) able to bind PI3K, PP2A and SHP-2 and one proline-rich motif able to bind SH3 containing proteins. One role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref. No.: P16410.3 (SEQ ID NO: 27) (homology herein may be determined using standard software such as BLAST or FASTA) or fragments thereof, and/or may optionally comprise up to one or up to two or up to three conservative amino acid substitutions.

SEQ ID NO: 27 is provided below:

(SEQ ID NO: 27)
MACLGFQRHKAQLNLATRTWPCTLLFFLLFIPVFCKAMHVAQPAWLASS
RGIASFVCEYASPGKATEVRVTVLRQADSQVTEVCAATYMMGNELTFLD
DSICTGTSSGNQLTIQGLRAMDTGLYICKVELMYPPPYYLGIGNGTQIY
VIDPEPCPDSDFLLWILAAVSSGLFFYSFLLTAVSLSKMLKKRSPLTTG
VYVKMPPTEPECEKQFQPYFIPIN

In accordance with the presently disclosed subject matter, a “CTLA-4 nucleic acid molecule” refers to a polynucleotide encoding a CTLA-4 polypeptide.

PD-1 is a negative immune regulator of activated T cells upon engagement with its corresponding ligands PD-L1 and PD-L2 expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, that PD-1 negatively regulates TCR signals. SHP-I and SHP-2 phosphatases bind to the cytoplasmic tail of PD-1 upon ligand binding. Upregulation of PD-L1 is one mechanism tumor cells may evade the host immune system. In pre-clinical and clinical trials, PD-1 blockade by antagonistic antibodies induced anti-tumor responses mediated through the host endogenous immune system. In accordance with the presently disclosed subject matter, a PD-1 polypeptide can have an amino acid sequence that is at least about 85%, about 90%, about 95%, about 96%, about 97%, about 98%, about 99% or about 100% homologous to NCBI Reference No: NP_005009.2 (SEQ ID NO: 28) or fragments thereof, and/or may optionally comprise up to one or up to two or up to three conservative amino acid substitutions.

SEQ ID NO: 28 is provided below:

(SEQ ID NO: 28)
MQIPQAPWPVVWAVLQLGWRPGWFLDSPDRPWNPPTFSPALLWTEGDNA
TFTCSFSNTSESFVLNWYRMSPSNQTDKLAAFPEDRSQPGQDCRFRVTQ
LPNGRDFHMSVVRARRNDSGTYLCGAISLAPKAQIKESLRAELRVTERR
AEVPTARPSPSPRPAGQFQTLVVGWGGLLGSLVLLVWVLAVICSRAARG
TIGARRTGQPLKEDPSAVPVFSVDYGELDFQWREKTPEPPVPCVPEQTE
YATIVFPSGMGTSSPARRGSADGPRSAQPLRPEDGHCSWPL

In accordance with the presently disclosed subject matter, a “PD-1 nucleic acid molecule” refers to a polynucleotide encoding a PD-1 polypeptide.

Lymphocyte-activation protein 3 (LAG-3) is a negative immune regulator of immune cells. LAG-3 belongs to the immunoglobulin (Ig) superfamily and contains 4 extracellular Ig-like domains. The LAG3 gene contains 8 exons. The sequence data, exon/intron organization, and chromosomal localization all indicate a close relationship of LAG3 to CD4. LAG3 has also been designated CD223 (cluster of differentiation 223).

In accordance with the presently disclosed subject matter, a LAG-3 polypeptide can have an amino acid sequence that is at least about 85%, about 90%, about 95%, about 96%, about 97%, about 98%, about 99% or about 100% homologous to UniProtKB/Swiss-Prot Ref. No.: P18627.5 (SEQ ID NO: 29) or fragments thereof, and/or may optionally comprise up to one or up to two or up to three conservative amino acid substitutions.

SEQ ID NO: 29 is provided below:

(SEQ ID NO: 29)
MWEAQFLGLLFLQPLWVAPVKPLQPGAEVPWWAQEGAPAQLPCSPTIPL
QDLSLLRRAGVTWQHQPDSGPPAAAPGHPLAPGPHPAAPSSWGPRPRRY
TVLSVGPGGLRSGRLPLQPRVQLDERGRQRGDFSLWLRPARRADAGEYR
AAVHLRDRALSCRLRLRLGQASMTASPPGSLRASDWVILNCSFSRPDRP
ASVHWFRNRGQGRVPVRESPHHHLAESFLFLPQVSPMDSGPWGCILTYR
DGFNVSIMYNLTVLGLEPPTPLTVYAGAGSRVGLPCRLPAGVGTRSFLT
AKWTPPGGGPDLLVTGDNGDFTLRLEDVSQAQAGTYTCHIHLQEQQLNA
TVTLAIITVTPKSFGSPGSLGKLLCEVTPVSGQERFVWSSLDTPSQRSF
SGPWLEAQEAQLLSQPWQCQLYQGERLLGAAVYFTELSSPGAQRSGRAP
GALPAGHLLLFLILGVLSLLLLVTGAFGFHLWRRQWRPRRFSALEQGIH
PPQAQSKIEELEQEPEPEPEPEPEPEPEPEPEQL

In accordance with the presently disclosed subject matter, a “LAG-3 nucleic acid molecule” refers to a polynucleotide encoding a LAG-3 polypeptide.

Natural Killer Cell Receptor 2B4 (2B4) mediates non-MHC restricted cell killing on NK cells and subsets of T cells. To date, the function of 2B4 is still under investigation, with the 2B4-S isoform believed to be an activating receptor, and the 2B4-L isoform believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref No.: Q9BZW8.2 (SEQ ID NO: 30) or fragments thereof, and/or may optionally comprise up to one or up to two or up to three conservative amino acid substitutions.

SEQ ID NO: 30 is provided below:

(SEQ ID NO: 30)
MLGQWTLILLLLLKVYQGKGCQGSADHWSISGVPLQLQPNSIQTKVDSI
AWKKLLPSQNGFHHILKWENGSLPSNTSNDRFSFIVKNLSLLIKAAQQQ
DSGLYCLEVTSISGKVQTATFQVFVFESLLPDKVEKPRLQGQGKILDRG
RCQVALSCLVSRDGNVSYAWYRGSKLIQTAGNLTYLDEEVDINGTHTYT
CNVSNPVSWESHTLNLTQDCQNAHQEFRFWPFLVIIVILSALFLGTLAC
FCVWRRKRKEKQSETSPKEFLTIYEDVKDLKTRRNHEQEQTFPGGGSTI
YSMIQSQSSAPTSQEPAYTLYSLIQPSRKSGSRKRNHSPSFNSTIYEVI
GKSQPKAQNPARLSRKELENFDVYS

In accordance with the presently disclosed subject matter, a “2B4 nucleic acid molecule” refers to a polynucleotide encoding a 2B4 polypeptide.

B- and T-lymphocyte attenuator (BTLA) expression is induced during activation of T cells, and BTLA remains expressed on Thl cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of human CD8⁺ cancer-specific T cells. BTLA has also been designated as CD272 (cluster of differentiation 272).

In accordance with the presently disclosed subject matter, a BTLA polypeptide can have an amino acid sequence that is at least about 85%>, about 90%, about 95%, about 96%, about 97%, about 98%, about 99% or about 100% homologous to UniProtKB/Swiss-Prot Ref. No.: Q7Z6A9.3 (SEQ ID NO: 31) or fragments thereof, and/or may optionally comprise up to one or up to two or up to three conservative amino acid substitutions.

SEQ ID NO: 31 is provided below:

(SEQ ID NO: 31)
MKTLPAMLGTGKLFWVFFLIPYLDIWNIHGKESCDVQLYIKRQSEHSIL
AGDPFELECPVKYCANRPHVTWCKLNGTTCVKLEDRQTSWKEEKNISFF
ILHFEPVLPNDNGSYRCSANFQSNLIESHSTTLYVTDVKSASERPSKDE
MASRPWLLYRLLPLGGLPLLITTCFCLFCCLRRHQGKQNELSDTAGREI
NLVDAHLKSEQTEASTRQNSQVLLSETGIYDNDPDLCFRMQEGSEVYSN
PCLEENKPGIVYASLNHSVIGPNSRLARNVKEAPTEYASICVRS

In accordance with the presently disclosed subject matter, a “BTLA nucleic acid molecule” refers to a polynucleotide encoding a BTLA polypeptide.

### Engineered Immune Cells of the Present Technology

As described herein, immune cells can be engineered to constitutively or conditionally express an anti-uPAR antigen binding fragment that binds to a uPAR antigen present on the cell surface of the senescent cells. The engineered immune cells of the present technology express a chimeric antigen receptor comprising an anti-uPAR antigen binding fragment (e.g., scFv) that permits delivery of the immune cell to the target senescent cells. In some embodiments, the engineered immune cells provided herein express a T-cell receptor (TCR) or other cell-surface ligand that binds to a uPAR antigen. In some embodiments, the T cell receptor is a chimeric T-cell receptor (CAR).

In exemplary embodiments provided herein, the engineered immune cells provided herein express a T-cell receptor (TCR) (e.g., a CAR) or other cell-surface ligand that binds to a uPAR antigen. In some embodiments, the engineered immune cells provided herein express a T-cell receptor (TCR) (e.g., a CAR) or other cell-surface ligand that binds to a uPAR antigen presented in the context of an MHC molecule. In some embodiments, the engineered immune cells provided herein express a T-cell receptor (TCR) (e.g., a CAR) or other cell-surface ligand that binds to a uPAR antigen presented in the context of an HLA-A2 molecule.

Provided herein are engineered immune cells (e.g., CAR T cells) that express a uPAR-specific antigen receptor, e.g., a chimeric antigen receptor, that effectively target senescent cells. The engineered immune cells (e.g., CAR T cells) provided herein that express a uPAR-specific antigen receptor, e.g., a chimeric antigen receptor, are useful in methods for eliminating senescent cells and treating or ameliorating the effects of senescence-associated pathologies, such as lung fibrosis, atherosclerosis, Alzheimer's disease, diabetes, liver fibrosis, chronic kidney disease, aging, or osteoarthritis, and/or treating cancer in a subject receiving senescence-inducing therapies (e.g., chemotherapeutic agents).

In certain embodiments, the engineered immune cells will proliferate extensively (e.g., 100 times or more) when it encounters a uPAR antigen at a tissue site, thus significantly increasing production of the chimeric antigen receptor comprising the anti-uPAR antigen binding fragment. The engineered immune cells (e.g., CAR T cells) can be generated by in vitro transduction of immune cells with a nucleic acid encoding the chimeric antigen receptor comprising the anti-uPAR antigen binding fragment. Further, the activity of the engineered immune cells (e.g., CAR T cells) can be adjusted by selection of co-stimulatory molecules included in the chimeric antigen receptor.

In some embodiments, the chimeric antigen receptor comprises a uPAR antigen binding fragment (e.g., scFv) comprising a V_{H}CDR1 sequence, a V_{H}CDR2 sequence, and a V_{H}CDR3 sequence of GFTFSNY (SEQ ID NO: 32), STGGGN (SEQ ID NO: 33), and QGGGYSDSFDY (SEQ ID NO:34); or GFSLSTSGM (SEQ ID NO: 35), WWDDD (SEQ ID NO: 36), and IGGSSGYMDY (SEQ ID NO: 37) respectively. Additionally or alternatively, in some embodiments, the uPAR antigen binding fragment (e.g., scFv) comprises a V_{L}CDR1 sequence, a V_{L}CDR2 sequence, and a V_{L}CDR3 sequence of KASKSISKYLA (SEQ ID NO: 38), SGSTLQS (SEQ ID NO: 39), and QQHNEYPLT (SEQ ID NO: 40); RASESVDSYGNSFMH (SEQ ID NO: 41), RASNLKS (SEQ ID NO: 42), and QQSNEDPWT (SEQ ID NO: 43); or KASENVVTYVS (SEQ ID NO: 44), GASNRYT (SEQ ID NO: 45), and GQGYSYPYT (SEQ ID NO: 46), respectively.

Additionally or alternatively, in some embodiments, the amino acid sequence of the V_{H} of the anti-uPAR antigen binding fragment (e.g., scFv) is:

(SEQ ID NO: 47)
EVQLVESGGGLVQPGRSLKLSCAASGFTFSNYAMAWVRQAPTKGLEWVA
SISTGGGNTYYRDSVKGRFTISRDNAKNTLYLQMDSLRSEDTATYYCAR
QGGGYSDSFDYWGQGVMVTVSS,
or
(SEQ ID NO: 48)
QVTLKESGPGILQPSQTLSLTCSFSGFSLSTSGMGVGWIRQPSGKGLEW
LAHIWWDDDKRYNPALKSRLTISKDPSSNQVFLKIASVDTADIATYYCV
RIGGSSGYMDYWGQGTSVTVSS.

Additionally or alternatively, in some embodiments, the amino acid sequence of the V_{L} of the anti-uPAR antigen binding fragment (e.g., scFv) is:

(SEQ ID NO: 49)
DVQMTQSPSNLAASPGESVSINCKASKSISKYLAWYQQKPGKANKLLIY
SGSTLQSGTPSRFSGSGSGTDFTLTIRNLEPEDFGLYYCQQHNEYPLTF
GSGTKLEIKR,
(SEQ ID NO: 50)
DIVLTQSPASLAVSLGQRATISCRASESVDSYGNSFMHWYQQKPGQPPK
LLIYRASNLKSGIPARFSGSGSGTDFTLTINPVEADDVATYCCQQSNED
PWTFGGGTKLEIKR,
or
(SEQ ID NO: 51)
NIVMTQSPKSMSMSVGERVTLTCKASENVVTYVSWYQQKPEQSPKLLIY
GASNRYTGVPDRFTGSGSATDFTLTISSVQAEDLADYHCGQGYSYPYTF
GGGTKLEIKR.

Additionally or alternatively, in some embodiments, the anti-uPAR antigen binding fragment (e.g., scFv) comprises an amino acid sequence selected from the group consisting of:

(SEQ ID NO: 52)
EVQLVESGGGLVQPGRSLKLSCAASGFTFSNYAMAWVRQAPTKGLEWVA
SISTGGGNTYYRDSVKGRFTISRDNAKNTLYLQMDSLRSEDTATYYCAR
QGGGYSDSFDYWGQGVMVTVSSGGGGSGGGGSGGGGSDVQMTQSPSNLA
ASPGESVSINCKASKSISKYLAWYQQKPGKANKLLIYSGSTLQSGTPSR
FSGSGSGTDFTLTIRNLEPEDFGLYYCQQHNEYPLTFGSGTKLEIKR;
(SEQ ID NO: 53)
QVTLKESGPGILQPSQTLSLTCSFSGFSLSTSGMGVGWIRQPSGKGLEW
LAHIWWDDDKRYNPALKSRLTISKDPSSNQVFLKIASVDTADIATYYCV
RIGGSSGYMDYWGQGTSVTVSSGGGGSGGGGSGGGGSDIVLTQSPASLA
VSLGQRATISCRASESVDSYGNSFMHWYQQKPGQPPKLLIYRASNLKSG
IPARFSGSGSGTDFTLTINPVEADDVATYCCQQSNEDPWTFGGGTKLEI
KR;
and
(SEQ ID NO: 54)
QVTLKESGPGILQPSQTLSLTCSFSGFSLSTSGMGVGWIRQPSGKGLEW
LAHIWWDDDKRYNPALKSRLTISKDPSSNQVFLKIASVDTADIATYYCV
RIGGSSGYMDYWGQGTSVTVSSGGGGSGGGGSGGGGSNIVMTQSPKSMS
MSVGERVTLTCKASENVVTYVSWYQQKPEQSPKLLIYGASNRYTGVPDR
FTGSGSATDFTLTISSVQAEDLADYHCGQGYSYPYTFGGGTKLEIKR.

Additionally or alternatively, in some embodiments, the anti-uPAR antigen binding fragment (e.g., scFv) comprises an amino acid sequence that has at least 80%, 85%, 90%, 95%, 96%, 97%, 98%, or 99% sequence identity to any one of SEQ ID NOs: 52-54. In some embodiments, the anti-uPAR antigen binding fragment (e.g., scFv) comprises an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NOs: 52-54. In some embodiments, the anti-uPAR antigen binding fragment is an scFv, a Fab, or a (Fab)₂.

Additionally or alternatively, in some embodiments, the anti-uPAR antigen binding fragment (e.g., scFv) is encoded by a nucleic acid sequence selected from the group consisting of:

(SEQ ID NO: 55)
GAAGTCCAACTCGTTGAAAGCGGCGGTGGTCTTGTCCAGCCAGGCAGAT
CACTGAAACTGTCATGCGCCGCCAGTGGCTTCACTTTCTCCAATTACGC
AATGGCGTGGGTTAGACAGGCCCCCACGAAAGGCTTGGAGTGGGTCGCA
TCAATCAGTACAGGAGGTGGAAACACTTACTATCGCGATAGTGTTAAGG
GGAGATTCACGATTAGCCGGGACAACGCGAAAAACACGTTGTATCTGCA
GATGGACTCACTTAGATCCGAGGACACAGCGACTTACTACTGTGCGAGG
CAGGGCGGAGGGTATAGTGATAGCTTTGATTACTGGGGCCAGGGCGTAA
TGGTAACTGTTAGTTCTGGTGGAGGTGGATCAGGTGGAGGTGGATCTGG
TGGAGGTGGATCTGATGTGCAGATGACACAGAGTCCTTCAAATTTGGCC
GCTTCACCCGGAGAATCAGTAAGTATCAACTGTAAAGCGTCCAAGTCCA
TTTCAAAGTATTTGGCATGGTATCAACAGAAGCCGGGAAAGGCGAACAA
ACTCCTGATTTATAGCGGGAGTACCTTGCAGTCCGGCACGCCTAGTAGA
TTTTCAGGCTCCGGTTCTGGGACCGACTTCACTTTGACGATTCGCAATT
TGGAACCAGAGGATTTTGGGCTGTACTATTGTCAGCAGCACAACGAATA
CCCGTTGACTTTTGGTAGTGGTACAAAGCTGGAAATCAAGAGAGCGGC
C;
(SEQ ID NO: 56)
CAGGTGACCCTGAAGGAGTCCGGCCCCGGCATCCTGCAGCCCAGCCAGA
CCCTGAGCCTGACCTGCTCCTTCAGCGGCTTCTCCCTGTCCACCTCCGG
CATGGGCGTGGGCTGGATCAGACAGCCCAGCGGCAAGGGCCTGGAGTGG
CTGGCCCACATCTGGTGGGACGATGACAAGAGATACAACCCCGCTCTGA
AGAGCCGGCTGACAATCAGCAAGGACCCTAGCAGTAACCAGGTGTTCCT
GAAGATCGCTTCCGTGGACACAGCAGACATCGCAACATACTATTGCGTG
CGGATCGGCGGAAGCAGTGGATACATGGACTACTGGGGACAGGGAACCA
GCGTGACCGTGAGCAGTGGTGGAGGTGGATCAGGTGGAGGTGGATCTGG
TGGAGGTGGATCTGACATCGTGCTGACCCAGAGCCCAGCTAGCTTGGCA
GTGAGCCTGGGACAGAGGGCTACCATCAGCTGCAGAGCTTCAGAGAGCG
TGGACAGCTACGGAAACAGCTTCATGCACTGGTACCAGCAGAAGCCAGG
ACAGCCACCTAAGCTGCTGATCTACCGGGCTAGCAACCTGAAGTCCGGA
ATCCCTGCTCGGTTTAGCGGAAGCGGTAGCGGCACCGACTTCACCCTGA
CAATCAACCCAGTGGAGGCCGACGATGTGGCAACCTACTGCTGTCAGCA
GAGCAACGAGGACCCATGGACCTTCGGCGGTGGAACCAAACTGGAGATC
AAGAGA;
and
(SEQ ID NO: 57)
CAGGTGACCCTGAAGGAGTCCGGCCCCGGCATCCTGCAGCCCAGCCAGA
CCCTGAGCCTGACCTGCTCCTTCAGCGGCTTCTCCCTGTCCACCTCCGG
CATGGGCGTGGGCTGGATCAGACAGCCCAGCGGCAAGGGCCTGGAGTGG
CTGGCCCACATCTGGTGGGACGATGACAAGAGATACAACCCCGCTCTGA
AGAGCCGGCTGACAATCAGCAAGGACCCTAGCAGTAACCAGGTGTTCCT
GAAGATCGCTTCCGTGGACACAGCAGACATCGCAACATACTATTGCGTG
CGGATCGGCGGAAGCAGTGGATACATGGACTACTGGGGACAGGGAACCA
GCGTGACCGTGAGCAGTGGTGGAGGTGGATCAGGTGGAGGTGGATCTGG
TGGAGGTGGATCTAACATCGTGATGACCCAGTCCCCTAAGAGCATGAGC
ATGAGCGTGGGCGAGAGAGTGACCCTGACCTGCAAAGCCTCCGAGAACG
TGGTGACCTACGTGAGCTGGTACCAGCAGAAGCCTGAGCAGAGCCCTAA
GCTGCTGATCTACGGCGCTTCCAACAGATACACCGGAGTGCCTGACAGA
TTCACCGGCAGCGGAAGCGCAACCGACTTCACCTTGACCATCAGCAGCG
TGCAGGCTGAGGACCTGGCCGACTACCACTGCGGCCAGGGCTACAGCTA
CCCTTACACCTTCGGTGGAGGCACCAAGCTGGAGATCAAGCGG.

Additionally or alternatively, in some embodiments, the anti-uPAR antigen binding fragment (e.g., scFv) is encoded by a nucleic acid sequence that has at least 80%, 85%, 90%, 95%, 96%, 97%, 98%, or 99% sequence identity to any one of SEQ ID NOs: 55-57. In some embodiments, the anti-uPAR antigen binding fragment (e.g., scFv) is encoded by a nucleic acid that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NOs: 55-57.

In some embodiments, the chimeric antigen receptor comprises a uPAR binding fragment (e.g., a uPA fragment) comprising the amino acid sequence:

(SEQ ID NO: 59)
MRALLARLLLCVLVVSDSKGSNELHQVPSNCDCLNGGTCVSNKYFSNIH
WCNCPKKFGGQHCEIDKSKTCYEGNGHFYRGKASTDTMGRPCLPWNSAT
VLQQTYHAHRSDALQLGLGKHNYCRNPDNRRRPWCYVQVGLKPLVQECM
VHDCADGKKP;
or
(SEQ ID NO: 60)
MRALLARLLLCVLVVSDSKGSNELHQVPSNCDCLNGGTCVSNKYFSNIH
WCNCPKKFGGQHCEIDKSKTCYEGNGHFYRGKASTDTMGRPCLPWNSAT
VLQQTYHAHRSDALQLGLGKHNYCRNPDNRRRPW.

Additionally or alternatively, in some embodiments, the uPAR binding fragment (e.g., uPa fragment) comprises an amino acid sequence that has at least 80%, 85%, 90%, 95%, 96%, 97%, 98%, or 99% sequence identity to SEQ ID NO: 59 or SEQ ID NO: 60. In some embodiments, the uPAR binding fragment (e.g., uPa fragment) comprises an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 59 or SEQ ID NO: 60.

Additionally or alternatively, in some embodiments, the uPAR binding fragment (e.g., a uPA fragment) is encoded by a nucleic acid sequence selected from the group consisting of:

(SEQ ID NO: 61)
ATGAGAGCCCTGCTGGCGCGCCTGCTTCTCTGCGTCCTGGTCGTGAGCG
ACTCCAAAGGCAGCAATGAACTTCATCAAGTTCCATCGAACTGTGACTG
TCTAAATGGAGGAACATGTGTGTCCAACAAGTACTTCTCCAACATTCAC
TGGTGCAACTGCCCAAAGAAATTCGGAGGGCAGCACTGTGAAATAGATA
AGTCAAAAACCTGCTATGAGGGGAATGGTCACTTTTACCGAGGAAAGGC
CAGCACTGACACCATGGGCCGGCCCTGCCTGCCCTGGAACTCTGCCACT
GTCCTTCAGCAAACGTACCATGCCCACAGATCTGATGCTCTTCAGCTGG
GCCTGGGGAAACATAATTACTGCAGGAACCCAGACAACCGGAGGCGACC
CTGGTGCTATGTGCAGGTGGGCCTAAAGCCGCTTGTCCAAGAGTGCATG
GTGCATGACTGCGCAGATGGAAAAAAGCCC;
or
(SEQ ID NO: 62)
ATGAGAGCCCTGCTGGCGCGCCTGCTTCTCTGCGTCCTGGTCGTGAGCG
ACTCCAAAGGCAGCAATGAACTTCATCAAGTTCCATCGAACTGTGACTG
TCTAAATGGAGGAACATGTGTGTCCAACAAGTACTTCTCCAACATTCAC
TGGTGCAACTGCCCAAAGAAATTCGGAGGGCAGCACTGTGAAATAGATA
AGTCAAAAACCTGCTATGAGGGGAATGGTCACTTTTACCGAGGAAAGGC
CAGCACTGACACCATGGGCCGGCCCTGCCTGCCCTGGAACTCTGCCACT
GTCCTTCAGCAAACGTACCATGCCCACAGATCTGATGCTCTTCAGCTGG
GCCTGGGGAAACATAATTACTGCAGGAACCCAGACAACCGGAGGCGACC
CTGG

Additionally or alternatively, in some embodiments, the uPAR binding fragment is encoded by a nucleic acid sequence that has at least 80%, 85%, 90%, 95%, 96%, 97%, 98%, or 99% sequence identity to any one of SEQ ID NOs: 61-62. In some embodiments, the uPAR binding fragment is encoded by a nucleic acid that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NOs: 61-62.

Additionally or alternatively, in certain embodiments, the uPAR-specific CAR of the present technology and a reporter or selection marker (e.g., GFP, LNGFR) are expressed as a single polypeptide linked by a self-cleaving linker, such as a P2A linker. In certain embodiments, the CAR and a reporter or selection marker (e.g., GFP, LNGFR) are expressed as two separate polypeptides.

In any and all of the preceding embodiments, the CAR comprises an extracellular binding fragment (e.g., anti-uPAR scFv or uPA fragment) that specifically binds to a uPAR antigen or polypeptide, a transmembrane domain comprising a CD28 polypeptide and/or a CD8 polypeptide, and an intracellular domain comprising a CD3ζ polypeptide and optionally a co-stimulatory signaling region disclosed herein. The CAR may also comprise a signal peptide or a leader sequence covalently joined to the N-terminus of the extracellular uPAR binding fragment. The signal peptide comprises amino acids having the sequence set forth in SEQ ID NO: 3, SEQ ID NO: 5, SEQ ID NO: 7 or SEQ ID NO: 9.

Additionally or alternatively, in some embodiments, the nucleic acid encoding the CAR of the present technology is operably linked to an inducible promoter. In some embodiments, the nucleic acid encoding the CAR of the present technology is operably linked to a constitutive promoter.

In some embodiments, the inducible promoter is a synthetic Notch promoter that is activatable in a CAR T cell, where the intracellular domain of the CAR contains a transcriptional regulator that is released from the membrane when engagement of the CAR with the uPAR antigen/polypeptide induces intramembrane proteolysis (see, e.g., Morsut et al., Cell 164(4): 780-791 (2016). Accordingly, further transcription of the uPAR-specific CAR is induced upon binding of the engineered immune cell with the uPAR antigen/polypeptide.

The presently disclosed subject matter also provides isolated nucleic acid molecules encoding the CAR constructs described herein or a functional portion thereof. In certain embodiments, the isolated nucleic acid molecule encodes an anti-uPAR-targeted CAR comprising (a) an uPAR binding fragment (e.g., an anti-uPAR scFv or uPA fragment) that specifically binds to a uPAR antigen, (b) a transmembrane domain comprising a CD8 polypeptide or CD28 polypeptide, and (c) an intracellular domain comprising a CD3ζ polypeptide, and optionally one or more of a co-stimulatory signaling region disclosed herein, a P2A self-cleaving peptide, and/or a reporter or selection marker (e.g., GFP, LNGFR) provided herein. The at least one co-stimulatory signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the isolated nucleic acid molecule encodes an uPAR-targeted CAR comprising a uPAR binding fragment (e.g., an anti-uPAR scFv or uPA fragment) that specifically binds to a uPAR antigen/polypeptide, fused to a synthetic Notch transmembrane domain and an intracellular cleavable transcription factor. In certain embodiments, the present disclosure provides an isolated nucleic acid molecule encoding a uPAR-specific CAR that is inducible by release of the transcription factor of a synthetic Notch system.

In certain embodiments, the isolated nucleic acid molecule encodes a functional portion of a presently disclosed CAR constructs. As used herein, the term “functional portion” refers to any portion, part or fragment of a CAR, which portion, part or fragment retains the biological activity of the parent CAR. For example, functional portions encompass the portions, parts or fragments of a uPAR-specific CAR that retains the ability to recognize a target senescent cell, to treat cancer or a senescence-associated pathology, to a similar, same, or even a higher extent as the parent CAR. In certain embodiments, an isolated nucleic acid molecule encoding a functional portion of a uPAR-specific CAR can encode a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent CAR.

The presently disclosed subject matter provides engineered immune cells expressing a uPAR-specific T-cell receptor (e.g., a CAR) or other ligand that comprises an extracellular antigen-binding domain, a transmembrane domain and an intracellular domain, where the extracellular antigen-binding domain specifically binds a uPAR antigen/polypeptide. In certain embodiments immune cells can be transduced with a presently disclosed CAR constructs such that the cells express the CAR. The presently disclosed subject matter also provides methods of using such cells for the treatment of cancer or senescence-associated pathology.

The engineered immune cells of the presently disclosed subject matter can be cells of the lymphoid lineage or myeloid lineage. The lymphoid lineage, comprising B, T, and natural killer (NK) cells, provides for the production of antibodies, regulation of the cellular immune system, detection of foreign agents in the blood, detection of cells foreign to the host, and the like. Non-limiting examples of immune cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T cells, and T6 T cells. Cytotoxic T cells (CTL or killer T cells) are a subset of T lymphocytes capable of inducing the death of infected somatic or tumor cells. In certain embodiments, the CAR-expressing T cells express Foxp3 to achieve and maintain a T regulatory phenotype.

Natural killer (NK) cells can be lymphocytes that are part of cell-mediated immunity and act during the innate immune response. NK cells do not require prior activation in order to perform their cytotoxic effect on target cells.

The engineered immune cells of the presently disclosed subject matter can express an extracellular uPAR binding domain (e.g., an anti-uPAR scFv, an anti-uPAR Fab that is optionally crosslinked, an anti-uPAR F(ab)₂ or a uPA fragment) that specifically binds to a uPAR antigen, for the treatment of cancer or a senescence-associated pathology. Such engineered immune cells can be administered to a subject (e.g., a human subject) in need thereof for the treatment of cancer or a senescence-associated pathology. In some embodiments, the immune cell is a lymphocyte, such as a T cell, a B cell or a natural killer (NK) cell. In certain embodiments, the engineered immune cell is a T cell. The T cell can be a CD4⁺ T cell or a CD8⁺ T cell. In certain embodiments, the T cell is a CD4⁺ T cell. In certain embodiments, the T cell is a CD8⁺ T cell.

The engineered immune cells of the present disclosure can further include at least one recombinant or exogenous co-stimulatory ligand. For example, the engineered immune cells of the present disclosure can be further transduced with at least one co-stimulatory ligand, such that the engineered immune cells co-expresses or is induced to co-express the uPAR-specific CAR and the at least one co-stimulatory ligand. The interaction between the uPAR-specific CAR and the at least one co-stimulatory ligand provides a non-antigen-specific signal important for full activation of an immune cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LT-α), lymphotoxin-beta (LT-0), CD257/B cell-activating factor (BAFF)/BLYS/THANK/TALL-1, glucocorticoid-induced TNF Receptor ligand (GITRL), TNF-related apoptosis-inducing ligand (TRAIL), and LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28, or PD-L1/(B7-H1) that are ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the engineered immune cell comprises one recombinant co-stimulatory ligand (e.g., 4-1BBL). In certain embodiments, the engineered immune cell comprises two recombinant co-stimulatory ligands (e.g., 4-1BBL and CD80). CARs comprising at least one co-stimulatory ligand are described in U.S. Pat. No. 8,389,282, which is incorporated by reference in its entirety.

Furthermore, the engineered immune cells of the present disclosure can further comprise at least one exogenous cytokine. For example, a presently disclosed engineered immune cell can be further transduced with at least one cytokine, such that the engineered immune cell secretes the at least one cytokine as well as expresses the uPAR-specific CAR. In certain embodiments, the at least one cytokine is selected from the group consisting of IL-2, IL-3, IL-6, IL-7, IL-11, IL-12, IL-15, IL-17, and IL-21.

The engineered immune cells can be generated from peripheral donor lymphocytes, e.g., those disclosed in Sadelain, M., et al., Nat Rev Cancer 3:35-45 (2003) (disclosing peripheral donor lymphocytes genetically modified to express CARs), in Morgan, R. A. et al., Science 314: 126-129 (2006) (disclosing peripheral donor lymphocytes genetically modified to express a full-length tumor antigen-recognizing T cell receptor complex comprising the α and β heterodimer), in Panelli et al., J Immunol 164:495-504 (2000); Panelli et al., J Immunol 164:4382-4392 (2000) (disclosing lymphocyte cultures derived from tumor infiltrating lymphocytes (TTLs) in tumor biopsies), and in Dupont et al., Cancer Res 65:5417-5427 (2005); Papanicolaou et al., Blood 102:2498-2505 (2003) (disclosing selectively inv/Yro-expanded antigen-specific peripheral blood leukocytes employing artificial antigen-presenting cells (AAPCs) or pulsed dendritic cells). The engineered immune cells (e.g., T cells) can be autologous, non-autologous (e.g., allogeneic), or derived in vitro from engineered progenitor or stem cells.

In certain embodiments, the engineered immune cells of the present disclosure (e.g., T cells) express from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers per cell of a presently disclosed uPAR-specific CAR.

For example, the higher the CAR expression level in an engineered immune cell, the greater cytotoxicity and cytokine production the engineered immune cell exhibits. An engineered immune cell (e.g., T cell) having a high uPAR-specific CAR expression level can induce antigen-specific cytokine production or secretion and/or exhibit cytotoxicity to a tissue or a cell having a low expression level of uPAR-specific CAR, e.g., about 2,000 or less, about 1,000 or less, about 900 or less, about 800 or less, about 700 or less, about 600 or less, about 500 or less, about 400 or less, about 300 or less, about 200 or less, about 100 or less of uPAR antigen binding sites/cell. Additionally or alternatively, the cytotoxicity and cytokine production of a presently disclosed engineered immune cell (e.g., T cell) are proportional to the expression level of uPAR antigen in a target tissue or a target cell. For example, the higher the expression level of uPAR antigen in the target, the greater cytotoxicity and cytokine production the engineered immune cell exhibits.

The unpurified source of immune cells may be any source known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cells initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. Suitably, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Usually, the cells are collected in a medium comprising 2% fetal calf serum (FCS) or 0.2% bovine serum albumin (BSA) or any other suitable (e.g., sterile), isotonic medium.

In some embodiments, the engineered immune cells comprise one or more additional modifications. For example, in some embodiments, the engineered immune cells comprise and express (are transduced to express) an antigen recognizing receptor that binds to a second antigen that is different than the first uPAR antigen. The inclusion of an antigen recognizing receptor in addition to a presently disclosed CAR on the engineered immune cell can increase the avidity of the CAR (or the engineered immune cell comprising the same) on a target cell, especially, the CAR is one that has a low binding affinity to a particular uPAR antigen, e.g., a K_{d} of about 2×10⁻⁸ M or more, about 5×10⁻⁸ M or more, about 8×10⁻⁸ M or more, about 9×10⁻⁸ M or more, about 1×10⁻⁷ M or more, about 2×10⁻⁷ M or more, or about 5×10⁻⁷ M or more.

In certain embodiments, the antigen recognizing receptor is a chimeric co-stimulatory receptor (CCR). CCR is described in Krause, et al., J. Exp. Med. 188(4):619-626(1998), and US20020018783, the contents of which are incorporated by reference in their entireties. CCRs mimic co-stimulatory signals, but unlike, CARs, do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with a CAR, can augment T-cell reactivity against the dual-antigen expressing cells, thereby improving selective targeting. Kloss et al., describe a strategy that integrates combinatorial antigen recognition, split signaling, and, critically, balanced strength of T-cell activation and costimulation to generate T cells that eliminate target cells that express a combination of antigens while sparing cells that express each antigen individually (Kloss et al., Nature Biotechnology 31(1):71-75 (2013)). With this approach, T-cell activation requires CAR-mediated recognition of one antigen, whereas costimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen. In certain embodiments, the CCR comprises (a) an extracellular antigen-binding domain that binds to an antigen different than the first uPAR antigen, (b) a transmembrane domain, and (c) a co-stimulatory signaling region that comprises at least one co-stimulatory molecule, including, but not limited to, CD28, 4-1BB, OX40, ICOS, PD-1, CTLA-4, LAG-3, 2B4, and BTLA. In certain embodiments, the co-stimulatory signaling region of the CCR comprises one co-stimulatory signaling molecule. In certain embodiments, the one co-stimulatory signaling molecule is CD28. In certain embodiments, the one co-stimulatory signaling molecule is 4-1BB. In certain embodiments, the co-stimulatory signaling region of the CCR comprises two co-stimulatory signaling molecules. In certain embodiments, the two co-stimulatory signaling molecules are CD28 and 4-1BB. A second antigen is selected so that expression of both the first uPAR antigen and the second antigen is restricted to the targeted cells (e.g., cancerous cells). Similar to a CAR, the extracellular antigen-binding domain can be an scFv, a Fab, a F(ab)₂; or a fusion protein with a heterologous sequence to form the extracellular antigen-binding domain. In certain embodiments, the CCR comprises an scFv that binds to CD138, transmembrane domain comprising a CD28 polypeptide, and a co-stimulatory signaling region comprising two co-stimulatory signaling molecules that are CD28 and 4-1BB.

In certain embodiments, the antigen recognizing receptor is a truncated CAR. A “truncated CAR” is different from a CAR by lacking an intracellular signaling domain. For example, a truncated CAR comprises an extracellular antigen-binding domain and a transmembrane domain, and lacks an intracellular signaling domain. In accordance with the presently disclosed subject matter, the truncated CAR has a high binding affinity to the second antigen expressed on the targeted cells. The truncated CAR functions as an adhesion molecule that enhances the avidity of a presently disclosed CAR, especially, one that has a low binding affinity to a uPAR antigen, thereby improving the efficacy of the presently disclosed CAR or engineered immune cell (e.g., T cell) comprising the same. In certain embodiments, the truncated CAR comprises an extracellular antigen-binding domain that binds to CD138, a transmembrane domain comprising a CD8 polypeptide. A presently disclosed T cell comprises or is transduced to express a presently disclosed CAR targeting uPAR antigen and a truncated CAR targeting CD138. In certain embodiments, the targeted cells are solid tumor cells. In some embodiments, the engineered immune cells are further modified to suppress expression of one or more genes. In some embodiments, the engineered immune cells are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the engineered immune cells are modified to disrupt or reduce expression of an endogenous T-cell receptor gene (see, e.g., WO 2014153470, which is incorporated by reference in its entirety). In some embodiments, the engineered immune cells are modified to result in disruption or inhibition of PD1, PDL-1 or CTLA-4 (see, e.g., U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

uPAR Immunoglobulin Compositions

In one aspect, the present disclosure also provides an antibody or an antigen binding fragment thereof comprising a variable heavy chain (V_{H}) and a variable light chain (V_{L}), wherein the V_{H} comprises a V_{H}CDR1 sequence, a V_{H}CDR2 sequence, and a V_{H}CDR3 sequence of GFSLSTSGM (SEQ ID NO: 35), WWDDD (SEQ ID NO: 36), and IGGSSGYMDY (SEQ ID NO: 37) respectively; and/or the V_{L} comprises a V_{L}CDR1 sequence, a V_{L}CDR2 sequence, and a V_{L}CDR3 sequence of: RASESVDSYGNSFMHI (SEQ ID NO: 41), RASNLKS (SEQ ID NO: 42), and QQSNEDPWT (SEQ ID NO: 43), respectively; or KASENVVTYVS (SEQ ID NO: 44), GASNRYT (SEQ ID NO: 45), and GQGYSYPYT (SEQ ID NO: 46), respectively. In some embodiments, the V_{H} comprises an amino acid sequence that is at least 90%, at least 95%, or 100% identical to SEQ ID NO: 48 and/or the V_{L} comprises an amino acid sequence that is at least 90%, at least 95%, or 100% identical to SEQ ID NO: 50 or SEQ ID NO: 51. In some embodiments, the antibody or antigen binding fragment specifically binds to uPAR. Additionally or alternatively, in some embodiments, the antibody or antigen binding fragment further comprises a Fc domain of an isotype selected from the group consisting of IgG1, IgG2, IgG3, IgG4, IgA1, IgA2, IgM, IgD, and IgE. In certain embodiments, the antigen binding fragment is selected from the group consisting of Fab, F(ab′)2, Fab′, scFv, and Fv. The antibody may be a monoclonal antibody, a chimeric antibody, a humanized antibody, or a bispecific antibody.

In one aspect, the present disclosure also provides a recombinant nucleic acid sequence encoding any of the antibodies or antigen binding fragments disclosed herein, as well as host cells and vectors comprising the same.

In another aspect, the present disclosure provides a composition comprising an antibody or antigen binding fragment of the present technology and a pharmaceutically-acceptable carrier, wherein the antibody or antigen binding fragment is optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

### Vectors

Many expression vectors are available and known to those of skill in the art and can be used for expression of polypeptides provided herein. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g., a hexa-his tag (SEQ ID NO: 64) or a myc tag, hemagglutinin tag or a tag for purification, for example, a GST fusion, and a sequence for directing protein secretion and/or membrane association.

Expression of antibodies or antigen binding fragments thereof can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310(1981)), the promoter contained in the 3′ long terminal repeat of Rous sarcoma virus (Yamamoto et al., Cell 22:787-797(1980)), the herpes thymidine kinase promoter (Wagner et al., Proc. Natl. Acad. Sci. USA 75: 1441-1445 (1981)), the regulatory sequences of the metallothionein gene (Brinster et al., Nature 296:39-42 (1982)); prokaryotic expression vectors such as the β-lactamase promoter (Jay et al., Proc. Natl. Acad. Sci. USA 75:5543 (1981)) or the tac promoter (DeBoer et al., Proc. Natl. Acad. Sci. USA 50:21-25(1983)); see also “Useful Proteins from Recombinant Bacteria”: in Scientific American 242:79-94 (1980)); plant expression vectors containing the nopaline synthetase promoter (Herrera-Estrella et al., Nature 505:209-213(1984)) or the cauliflower mosaic virus 35S RNA promoter (Gardner et al., Nucleic Acids Res. 9:2871(1981)), and the promoter of the photosynthetic enzyme ribulose bisphosphate carboxylase (Herrera-Estrella et al., Nature 510: 1 15-120(1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409(1986); MacDonald, Hepatology 7:425-515 (1987)); insulin gene control region which is active in pancreatic beta cells (Hanahan et al., Nature 515: 115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7: 1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al., Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al., Science 254: 1372-1378 (1986)).

In addition to the promoter, the expression vector typically contains a transcription unit or expression cassette that contains all the additional elements required for the expression of an antibody, or antigen binding fragment thereof, in host cells. A typical expression cassette contains a promoter operably linked to the nucleic acid sequence encoding the polypeptide chains of interest and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of engineered immune cells (e.g., T cells, NK cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the uPAR-specific CAR can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide uPAR-specific CAR expressing cells, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al., Mol. Cell. Biol. 5:431-437 (1985)); PA317 (Miller, et al., Mol. Cell. Biol. 6:2895-2902 (1986)); and CRIP (Danos, et al. Proc. Natl. Acad. Sci. USA 85:6460-6464 (1988)). Non-amphotropic particles are suitable too, e.g., particles pseudotyped with VSVG, RD 114 or GALV envelope and any other known in the art.

Possible methods of transduction also include direct co-culture of the cells with producer cells, e.g., by the method of Bregni, et al., Blood 80: 1418-1422(1992), or culturing with viral supernatant alone or concentrated vector stocks with or without appropriate growth factors and polycations, e.g., by the method of Xu, et al., Exp. Hemat. 22:223-230 (1994); and Hughes, et al., J. Clin. Invest. 89: 1817 (1992).

Transducing viral vectors can be used to express a co-stimulatory ligand and/or secretes a cytokine (e.g., 4-1BBL and/or IL-12) in an engineered immune cell. In some embodiments, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649, 1997; Naldini et al., Science 272:263 267 (1996); and Miyoshi et al., Proc. Natl. Acad. Sci. U.S.A. 94: 10319, (1997)). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244: 1275-1281 (1989); Eglitis et al., BioTechniques 6:608-614, (1988); Tolstoshev et al., Current Opinion in Biotechnology 1:55-61(1990); Sharp, The Lancet 337: 1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med 323:370 (1990); Anderson et al., U.S. Pat. No. 5,399,346).

In certain non-limiting embodiments, the vector expressing a presently disclosed uPAR-specific CAR is a retroviral vector, e.g., an oncoretroviral vector.

Non-viral approaches can also be employed for the expression of a protein in a cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J Med. Sci. 298:278, (1989); Staubinger et al., Methods in Enzymology 101:512 (1983)), asialoorosomucoid-polylysine conjugation (Wu et al., Journal of Biological Chemistry 263: 14621 (1988); Wu et al., Journal of Biological Chemistry 264: 16985 (1989)), or by micro-injection under surgical conditions (Wolff et al., Science 247: 1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor la enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### Polypeptides and Analogs and Polynucleotides

Also included in the presently disclosed subject matter are polypeptides including extracellular antigen-binding fragments that specifically bind to a uPAR antigen (e.g., a human uPAR antigen) (e.g., an scFv (e.g., a human scFv), a Fab, or a (Fab)₂), CD3ζ, CD8, CD28, etc. or fragments thereof, and polynucleotides encoding the same, that are modified in ways that enhance their biological activity when expressed in an engineered immune cell. The presently disclosed subject matter provides methods for optimizing an amino acid sequence or a nucleic acid sequence by producing an alteration in the sequence. Such alterations may comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%, about 98%, about 99% or more identity or homology with all or part of a naturally-occurring amino acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program may be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethyl sulfate or by site-specific mutagenesis as described in Sambrook, Fritsch and Maniatis, Molecular Cloning: A Laboratory Manual (2nd ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the present technology. Such analogs are administered according to methods of the presently disclosed subject matter. Such analogs may exceed the physiological activity of the original polypeptide. Methods of analog design are well known in the art, and synthesis of analogs can be carried out according to such methods by modifying the chemical structures such that the resultant analogs increase the antineoplastic activity of the original polypeptide when expressed in an engineered immune cell. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding an extracellular antigen-binding fragment that specifically binds to a uPAR antigen (e.g., human uPAR antigen) (e.g., an scFv (e.g., a human scFv), a Fab, or a (Fab)₂), CD3, CD8, CD28 can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

### Administration

Engineered immune cells expressing the uPAR-specific CAR of the presently disclosed subject matter can be provided systemically or directly to a subject for treating cancer or a senescence-associated pathology. In certain embodiments, engineered immune cells are directly injected into an organ of interest (e.g., an organ affected by a senescence-associated pathology). Additionally or alternatively, the engineered immune cells are provided indirectly to the organ of interest, for example, by administration into the circulatory system (e.g., the tumor vasculature) or into the tissue of interest (e.g., solid tumor). Expansion and differentiation agents can be provided prior to, during or after administration of cells and compositions to increase production of T cells in vitro or in vivo.

Engineered immune cells of the presently disclosed subject matter can be administered in any physiologically acceptable vehicle, systemically or regionally, normally intravascularly, intraperitoneally, intrathecally, or intrapleurally, although they may also be introduced into bone or other convenient site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising engineered immune cells can comprise a purified population of cells. Those skilled in the art can readily determine the percentage of engineered immune cells in a cell population using various well-known methods, such as fluorescence activated cell sorting (FACS). The ranges of purity in cell populations comprising engineered immune cells can be from about 50% to about 55%, from about 55% to about 60%, about 60% to about 65%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The engineered immune cells can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., 7-interferon.

In certain embodiments, compositions of the presently disclosed subject matter comprise pharmaceutical compositions comprising engineered immune cells expressing a uPAR-specific CAR with a pharmaceutically acceptable carrier. Administration can be autologous or non-autologous. For example, engineered immune cells expressing a uPAR-specific CAR and compositions comprising the same can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising engineered immune cells expressing a uPAR-specific CAR), it can be formulated in a unit dosage injectable form (solution, suspension, emulsion).

### Formulations

Engineered immune cells expressing a uPAR-specific CAR and compositions comprising the same can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising engineered immune cells, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the engineered immune cells of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is suitable particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the engineered immune cells as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the engineered immune cells of the presently disclosed subject matter is the quantity of cells necessary to achieve an optimal effect. The quantity of cells to be administered will vary for the subject being treated. In certain embodiments, from about 10² to about 10¹², from about 10³ to about 10¹¹, from about 10⁴ to about 10¹⁰, from about 10⁵ to about 10⁹, or from about 10⁶ to about 10⁸ engineered immune cells of the presently disclosed subject matter are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more engineered immune cells of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. Generally, engineered immune cells are administered at doses that are nontoxic or tolerable to the patient.

The skilled artisan can readily determine the amount of cells and optional additives, vehicles, and/or carrier in compositions to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Therapeutic Uses of the Engineered Immune Cells of the Present Technology

For treatment, the amount of the engineered immune cells provided herein administered is an amount effective in producing the desired effect, for example, treatment or amelioration of the effects of cancer and senescence-associated pathologies, such as lung fibrosis, atherosclerosis, Alzheimer's disease, diabetes, osteoarthritis, liver fibrosis, chronic kidney disease, aging, or one or more symptoms thereof. An effective amount can be provided in one or a series of administrations of the engineered immune cells provided herein. An effective amount can be provided in a bolus or by continuous perfusion. For adoptive immunotherapy using antigen-specific T cells, while cell doses in the range of about 10⁶ to about 10¹⁰ are typically infused, lower doses of the engineered immune cells may be administered, e.g., about 10⁴ to about 10⁸.

Upon administration of the engineered immune cells into the subject, the engineered immune cells are induced that are specifically directed against a uPAR antigen. The engineered immune cells of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the engineered immune cells and the compositions comprising the same are intravenously administered to the subject in need. Methods for administering cells for adoptive cell therapies, including, for example, donor lymphocyte infusion and CAR T cell therapies, and regimens for administration are known in the art and can be employed for administration of the engineered immune cells provided herein.

The presently disclosed subject matter provides various methods of using the engineered immune cells (e.g., T cells) provided herein, expressing a uPAR-specific receptor (e.g., a CAR).

For example, the presently disclosed subject matter provides methods of reducing tumor burden in a subject. In one non-limiting example, the method of reducing tumor burden comprises administering an effective amount of the presently disclosed engineered immune cells to the subject and administering a suitable antibody targeted to the tumor, thereby inducing tumor cell death in the subject. In some embodiments, the engineered immune cells and the antibody are administered at different times. For example, in some embodiments, the engineered immune cells are administered and then the antibody is administered. In some embodiments, the antibody is administered 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 26 hours, 48 hours or more than 48 hours after the administration of the engineered immune cells of the present technology.

The presently disclosed engineered immune cells can reduce the number of tumor cells, reduce tumor size, and/or eradicate the tumor in the subject. In certain embodiments, the method of reducing tumor burden comprises administering an effective amount of engineered immune cells to the subject, thereby inducing tumor cell death in the subject. Non-limiting examples of suitable tumors include breast cancer, endometrial cancer, ovarian cancer, colon cancer, lung cancer, stomach cancer, prostate cancer, renal cancer, pancreatic cancer, acute lymphoblastic leukemia (ALL), chronic lymphocytic leukemia (CLL), acute myeloid leukemia (AML), and metastases thereof. In some embodiments, the cancer is a relapsed or refractory cancer. In some embodiments, the cancer is resistant to one or more cancer therapies, e.g., one or more chemotherapeutic drugs.

The presently disclosed subject matter also provides methods of increasing or lengthening survival of a subject with cancer (e.g., a tumor). In one non-limiting example, the method of increasing or lengthening survival of a subject with cancer (e.g., a tumor) comprises administering an effective amount of the presently disclosed engineered immune cell to the subject, thereby increasing or lengthening survival of the subject. The presently disclosed subject matter further provides methods for treating or preventing cancer (e.g., a tumor) in a subject, comprising administering the presently disclosed engineered immune cells to the subject. Also provided herein are methods for treating of inhibiting tumor growth or metastasis in a subject comprising contacting a tumor cell with an effective amount of any of the engineered immune cells provided herein.

Cancers whose growth may be inhibited using the engineered immune cells of the presently disclosed subject matter include cancers typically responsive to immunotherapy. Non-limiting examples of cancers for treatment include breast cancer, endometrial cancer, ovarian cancer, colon cancer, lung cancer, stomach cancer, prostate cancer, renal cancer, pancreatic cancer, acute lymphoblastic leukemia (ALL), chronic lymphocytic leukemia (CLL), acute myeloid leukemia (AML), and metastases thereof. In certain embodiments, the cancer is triple negative breast cancer or ovarian cancer. In some embodiments, the cancer is prostate cancer. In some embodiments, the cancer is ovarian cancer, non-small cell lung cancer, gastric cancer, colon cancer, or triple negative breast cancer.

Additionally, the presently disclosed subject matter provides methods of increasing immune-activating cytokine production in response to a cancer cell in a subject in need thereof. In one non-limiting example, the method comprises administering the presently disclosed engineered immune cell to the subject. The immune-activating cytokine can be granulocyte macrophage colony stimulating factor (GM-CSF), IFNα, IFN-β, IFN-γ, TNF-α, IL-2, IL-3, IL-6, IL-11, IL-7, IL-12, IL-15, IL-21, interferon regulatory factor 7 (IRF7), and combinations thereof. In certain embodiments, the engineered immune cells including a uPAR antigen-specific CAR of the presently disclosed subject matter increase the production of GM-CSF, IFN-γ, and/or TNF-α.

Suitable human subjects for therapy typically comprise two treatment groups that can be distinguished by clinical criteria. Subjects with “advanced disease” or “high tumor burden” are those who bear a clinically measurable tumor (e.g., multiple myeloma). A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor (e.g., multiple myeloma).

Another group of suitable subjects is known in the art as the “adjuvant group.” These are individuals who have had a history of neoplasia, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes. Another group has a genetic predisposition to neoplasia but has not yet evidenced clinical signs of neoplasia. For instance, women testing positive for a genetic mutation associated with breast cancer, but still of childbearing age, can wish to receive one or more of the engineered immune cells described herein in treatment prophylactically to prevent the occurrence of neoplasia until it is suitable to perform preventive surgery.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the uPAR-specific CAR-expressing engineered immune cells (e.g., T cells) to avert or minimize the risks of immunological complications (known as “malignant T-cell transformation”), e.g., graft versus-host disease (GvHD). Modification of the engineered immune cells can include engineering a suicide gene into the uPAR-specific CAR-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the C-terminus of the intracellular domain of the uPAR-specific CAR. The suicide gene can be included within the vector comprising nucleic acids encoding the presently disclosed uPAR-specific CARs. The incorporation of a suicide gene into the a presently disclosed uPAR-specific CAR gives an added level of safety with the ability to eliminate the majority of CAR T cells within a very short time period. A presently disclosed engineered immune cell (e.g., a T cell) incorporated with a suicide gene can be pre-emptively eliminated at a given time point post CAR T cell infusion, or eradicated at the earliest signs of toxicity.

In another aspect, the present disclosure provides methods for treating or ameliorating the effects of a senescence-associated pathology in a subject in need thereof comprising administering to the subject an effective amount of any of the engineered immune cells described herein, wherein the subject exhibits an increased accumulation of senescent cells compared to that observed in a healthy control subject. In some embodiments, the senescence-associated pathology is lung fibrosis, atherosclerosis, Alzheimer's disease, diabetes, liver fibrosis, chronic kidney disease, aging, or osteoarthritis. Additionally or alternatively, in certain embodiments, the senescent cells exhibit a Senescence-Associated Secretory Phenotype (SASP). The Senescence-Associated Secretory Phenotype may be induced by replication, an oncogene (e.g., HRAS^{G12D} NRAS^{G2D} NRAS^{G12D; D3A} etc.) or a drug (e.g., Cdk4/6 inhibitors, MEK inhibitors, doxorubicin etc.).

Examples of MEK inhibitors include trametinib, cobimetinib, binimetinib, selumetinib, PD-325901, TAK-733, CI-1040 (PD184352), PD0325901, MEK162, AZD8330, GDC-0623, refametinib, pimasertib, RO4987655, RO5126766, WX-554, HL-085, CInQ-03, G-573, PD184161, PD318088, PD98059, RO5068760, U0126, and SL327. Examples of CDK4/6 inhibitors include palbociclib, ribociclib, and abemaciclib. The properties, efficacy, and therapeutic indications of the various MEK inhibitors are described in Cheng & Tian, Molecules 22, 1551 (2017).

### Combination Therapy

Also provided are methods for treating cancer in a subject in need thereof comprising administering to the subject an effective amount of any of the engineered immune cells provided herein and a tumor specific monoclonal antibody, wherein the subject is receiving/has received senescence-inducing therapies (e.g., chemotherapeutic agents). In some embodiments, the tumor specific monoclonal antibody is administered subsequent to administration of the engineered immune cells. Examples of specific senescence-inducing therapies include, but are not limited to, doxorubicin, ionizing radiation therapy, combination therapy with MEK inhibitors and CDK4/6 inhibitors, combination therapy with CDCl₇ inhibitors and mTOR inhibitors, and the like. Examples of CDK4/6 inhibitors include palbociclib, ribociclib, and abemaciclib. Examples of MEK inhibitors include trametinib, cobimetinib, binimetinib, selumetinib, PD-325901, TAK-733, CI-1040 (PD184352), PD0325901, MEK162, AZD8330, GDC-0623, refametinib, pimasertib, RO4987655, RO5126766, WX-554, HL-085, CInQ-03, G-573, PD184161, PD318088, PD98059, RO5068760, U0126, and SL327. Examples of mTOR inhibitors include rapamycin, sertraline, sirolimus, everolimus, temsirolimus, ridaforolimus, and deforolimus. Examples of CDCl₇ inhibitors include TAK-931, PHA-767491, XL413, 1H-pyrrolo[2,3-b]pyridines, 2,3-dihydrothieno[3,2-d]pyrimidin-4(1H)-ones, furanone derivatives, trisubstituted thiazoles, pyrrolopyridinones, and the like.

Also provided herein are methods for treating of inhibiting tumor growth or metastasis in a subject comprising contacting a tumor cell with an effective amount of any of the engineered immune cells provided herein and a tumor specific monoclonal antibody. In some embodiments, the tumor specific monoclonal antibody is administered subsequent to administration of the engineered immune cells.

In some embodiments of the methods disclosed herein, the engineered immune cell(s) are administered are administered intravenously, intratumorally, intraperitoneally, subcutaneously, intramuscularly, or intratumorally. In some embodiments, the cancer or tumor is selected from among breast cancer, endometrial cancer, ovarian cancer, colon cancer, lung cancer, stomach cancer, prostate cancer, renal cancer, pancreatic cancer, acute lymphoblastic leukemia (ALL), chronic lymphocytic leukemia (CLL), acute myeloid leukemia (AML), and metastases thereof. In some embodiments, the subject is human.

Additionally or alternatively, in some embodiments, the methods of the present technology further comprise administering to the subject an additional cancer therapy. In some embodiments, the additional cancer therapy is selected from among chemotherapy, radiation therapy, immunotherapy, monoclonal antibodies, anti-cancer nucleic acids or proteins, anti-cancer viruses or microorganisms, and any combinations thereof. In some embodiments, the methods further comprise administering a cytokine to the subject. In some embodiments, the cytokine is administered prior to, during, or subsequent to administration of the one or more engineered immune cells. In some embodiments, the cytokine is selected from the group consisting of interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3C_{L}1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

The methods for treating cancer may further comprise sequentially, separately, or simultaneously administering to the subject at least one chemotherapeutic agent selected from the group consisting of nitrogen mustards, ethylenimine derivatives, alkyl sulfonates, nitrosoureas, gemcitabine, triazenes, folic acid analogs, anthracyclines, taxanes, COX-2 inhibitors, pyrimidine analogs, purine analogs, antibiotics, enzyme inhibitors, epipodophyllotoxins, platinum coordination complexes, vinca alkaloids, substituted ureas, methyl hydrazine derivatives, adrenocortical suppressants, hormone antagonists, endostatin, taxols, camptothecins, SN-38, doxorubicin, doxorubicin analogs, antimetabolites, alkylating agents, antimitotics, anti-angiogenic agents, tyrosine kinase inhibitors, mTOR inhibitors, heat shock protein (HSP90) inhibitors, proteosome inhibitors, HDAC inhibitors, pro-apoptotic agents, methotrexate and CPT-11.

Methods for treating lung fibrosis may further comprise sequentially, separately, or simultaneously administering to the subject at least one additional therapy selected from among pirfenidone, nintedanib, oxygen therapy, corticosteroids (e.g., prednisone), mycophenolate mofetil/mycophenolic acid, and azathioprine.

Methods for treating atherosclerosis may further comprise sequentially, separately, or simultaneously administering to the subject at least one additional therapy selected from among statins (e.g., Atorvastatin, Fluvastatin, Lovastatin, Pitavastatin, Pravastatin, Rosuvastatin calcium, Simvastatin), fibrates (e.g., Gemfibrozil, Fenofibrate), niacin, ezetimibe, bile acid sequestrants (e.g., cholestyramine, colestipol, colesevelam), proprotein convertase subtilisin kexin type 9 (PCSK9) inhibitors, anti-platelet medications (e.g., aspirin, Clopidogrel, Ticagrelor, warfarin, prasugral), beta blockers, Angiotensin-converting enzyme (ACE) inhibitors, calcium channel blockers, and diuretics.

Methods for treating Alzheimer's disease may further comprise sequentially, separately, or simultaneously administering to the subject at least one additional therapy selected from among donepezil, galantamine, memantine, rivastigmine, memantine extended-release and donepezil (Namzaric), aducanumab, solanezumab, insulin, verubecestat, AADvac1, CSP-1103, and intepirdine.

Methods for treating diabetes may further comprise sequentially, separately, or simultaneously administering to the subject at least one additional therapy selected from among insulin, metformin, amylin analogs, glucagon, sulfonylureas (e.g., glimepiride, glipizide, glyburide, chlorpropamide, tolazamide, tolbutamide), meglitinides (e.g., nateglinide, repaglinide), thiazolidinediones (e.g., pioglitazone, rosiglitazone), alpha-glucosidase inhibitors (e.g., acarbose, miglitol), dipeptidyl peptidase (DPP-4) inhibitors (e.g., alogliptin, linagliptin, sitagliptin, saxagliptin), sodium-glucose co-transporter 2 (SGLT2) inhibitors (e.g., canagliflozin, dapagliflozin, empagliflozin, ertugliflozin), and incretin mimetics (e.g., exenatide, liraglutide, dulaglutide, lixisenatide, semaglutide).

Methods for treating osteoarthritis may further comprise sequentially, separately, or simultaneously administering to the subject at least one additional therapy selected from among analgesics (e.g., acetaminophen, tramadol, oxycodone, hydrocodone), nonsteroidal anti-inflammatory drugs (e.g., aspirin, ibuprofen, naproxen, celecoxib), cyclooxygenase-2 inhibitors, corticosteroids, and hyaluronic acid.

Methods for treating liver fibrosis may further comprise sequentially, separately, or simultaneously administering to the subject at least one additional therapy selected from among ACE inhibitors (e.g., benazepril, Lisinopril, Ramipril), α-Tocopherol, interferon-α, PPAR-antagonists, colchicine, corticosteroids, endothelin inhibitors, interleukin-10, pentoxifylline, phosphatidylcholine, S-adenosyl-methionine, and TGF-β1 inhibitors.

Methods for treating chronic kidney disease may further comprise sequentially, separately, or simultaneously administering to the subject at least one additional therapy selected from among ACE inhibitors (e.g., benazepril, Lisinopril, Ramipril), statins (e.g., Atorvastatin, Fluvastatin, Lovastatin, Pitavastatin, Pravastatin, Rosuvastatin calcium, Simvastatin), furosemide, erythropoietin, phosphate binders (e.g., calcium acetate, calcium carbonate), colecalciferol, ergocalciferol, and cyclophosphamide.

### Diagnostic and Prognostic Methods of the Present Technology

Because not all patient populations suffering from diabetes, chronic kidney disease, or cardiovascular disease manifest elevated soluble uPAR (suPAR) levels (compared to healthy control populations), uPAR does not serve as an accurate biomarker for these disease states. See Eapen, D. J. et al., J Am Heart Assoc. 3 (2014); Hayek, S. S. et al., N Engl J Med. 373; 1916-1925 (2015); Theilade, S. et al., J Intern Med. 277:362-371 (2015). However, as described in the Examples herein, uPAR may be used as a marker to detect the senescent cell burden of a subject.

In one aspect, the present disclosure provides a method for detecting senescent cells in a biological sample obtained from a patient comprising: detecting the presence of senescent cells in the biological sample by detecting uPAR and/or suPAR polypeptide levels in the biological sample that are increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 100% compared to that observed in a reference sample. Alternatively, the present disclosure provides a method for detecting senescent cells in a biological sample obtained from a patient comprising: detecting the presence of senescent cells in the biological sample by detecting uPAR and/or suPAR polypeptide levels in the biological sample that are increased by at least 0.5-fold, at least 1.0 fold, at least 1.5-fold, at least 2.0 fold, at least 2.5-fold, at least 3.0 fold, at least 3.5-fold, at least 4.0 fold, at least 4.5-fold, at least 5.0 fold, at least 5.5-fold, at least 6.0 fold, at least 6.5-fold, at least 7.0 fold, at least 7.5-fold, at least 8.0 fold, at least 8.5-fold, at least 9.0 fold, at least 9.5-fold, or at least 10.0 fold compared to that observed in a reference sample. The reference sample may be obtained from a healthy control subject or may contain a predetermined level of the uPAR and/or suPAR polypeptide. The biological sample may be mucus, saliva, bronchial alveolar lavage (BAL), bronchial wash (BW), whole blood, cerebrospinal fluid (CSF), urine, plasma, serum, lymph, semen, synovial fluid, tears, amniotic fluid, bile, aqueous humor, or a bodily fluid. Additionally or alternatively, in some embodiments, the uPAR and/or suPAR polypeptide levels are detected via Western Blotting, flow cytometry, Enzyme-linked immunosorbent assay (ELISA), immunoprecipitation, immunoelectrophoresis, immunostaining, isoelectric focusing, High-performance liquid chromatography (HPLC), or mass-spectrometry.

In another aspect, the present disclosure provides a method for detecting the presence of a senescent preneoplastic lesion in a patient in need thereof comprising: (a) detecting uPAR and/or soluble uPAR (suPAR) polypeptide levels in a first biological sample obtained from the patient at a first time point; (b) detecting uPAR and/or soluble uPAR (suPAR) polypeptide levels in a second biological sample obtained from the patient at a second time point, wherein the second time point occurs after the first time point; and (c) detecting the presence of a senescent preneoplastic lesion in the patient when the uPAR and/or suPAR polypeptide levels in the second biological sample are increased compared to that observed in the first biological sample. In some embodiments, the senescent preneoplastic lesion is capable of promoting tumorigenesis (such as PanIN in pancreatic ductal adenocarcinoma (PDAC), adenomas in non-small cell lung cancer (NSCLC) and colorectal cancer (CRC), and nevi in melanoma).

In one aspect, the present disclosure provides a method for determining the efficacy of a senescence-inducing therapy in a patient in need thereof comprising: detecting uPAR and/or soluble uPAR (suPAR) polypeptide levels in a test biological sample obtained from the patient after administration of the senescence-inducing therapy, wherein the senescence-inducing therapy is effective when the uPAR and/or suPAR polypeptide levels in the test biological sample are elevated compared to that observed in a control biological sample obtained from the patient prior to administration of the senescence-inducing therapy. In some embodiments, the patient is suffering from or has been diagnosed with a senescence-associated pathology such as cancer, lung fibrosis, atherosclerosis, Alzheimer's disease, diabetes, osteoarthritis, liver fibrosis, or chronic kidney disease. Additionally or alternatively, in some embodiments, the senescence-inducing therapy includes the use of a chemotherapeutic agent and/or a targeted immunotherapy. Additionally or alternatively, in some embodiments, the method further comprises selecting the patient for treatment with an engineered immune cell that specifically targets uPAR (e.g., CAR T cells of the present technology) when the uPAR and/or suPAR polypeptide levels in the test biological sample are elevated compared to that observed in the control biological sample. In any of the preceding embodiments of the methods disclosed herein, the uPAR and/or suPAR polypeptide levels in the test biological sample are elevated by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 100% compared to that observed in the control biological sample. In other embodiments, the uPAR and/or suPAR polypeptide levels in the test biological sample are elevated by at least 0.5-fold, at least 1.0 fold, at least 1.5-fold, at least 2.0 fold, at least 2.5-fold, at least 3.0 fold, at least 3.5-fold, at least 4.0 fold, at least 4.5-fold, at least 5.0 fold, at least 5.5-fold, at least 6.0 fold, at least 6.5-fold, at least 7.0 fold, at least 7.5-fold, at least 8.0 fold, at least 8.5-fold, at least 9.0 fold, at least 9.5-fold, or at least 10.0 fold compared to that observed in the control biological sample. Additionally or alternatively, in some embodiments, the uPAR and/or suPAR polypeptide levels are detected via Western Blotting, flow cytometry, Enzyme-linked immunosorbent assay (ELISA), immunoprecipitation, immunoelectrophoresis, immunostaining, isoelectric focusing, High-performance liquid chromatography (HPLC), or mass-spectrometry.

In another aspect, the present disclosure provides a method for determining the efficacy of a senolytic CAR T cell therapy in a patient in need thereof comprising: detecting uPAR and/or soluble uPAR (suPAR) polypeptide levels in a test biological sample obtained from the patient after administration of the senolytic CAR T cell therapy, wherein the senolytic CAR T cell therapy is effective when the uPAR and/or suPAR polypeptide levels in the test biological sample are reduced compared to that observed in a control biological sample obtained from the patient prior to administration of the senolytic CAR T cell therapy. In some embodiments, the patient is suffering from or has been diagnosed with a senescence-associated pathology such as cancer, lung fibrosis, atherosclerosis, Alzheimer's disease, diabetes, osteoarthritis, liver fibrosis, or chronic kidney disease. In any of the preceding embodiments of the methods disclosed herein, the uPAR and/or suPAR polypeptide levels in the test biological sample are reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 100% compared to that observed in the control biological sample. In other embodiments, the uPAR and/or suPAR polypeptide levels in the test biological sample are reduced by at least 0.5-fold, at least 1.0 fold, at least 1.5-fold, at least 2.0 fold, at least 2.5-fold, at least 3.0 fold, at least 3.5-fold, at least 4.0 fold, at least 4.5-fold, at least 5.0 fold, at least 5.5-fold, at least 6.0 fold, at least 6.5-fold, at least 7.0 fold, at least 7.5-fold, at least 8.0 fold, at least 8.5-fold, at least 9.0 fold, at least 9.5-fold, or at least 10.0 fold compared to that observed in the control biological sample. Additionally or alternatively, in some embodiments, the uPAR and/or suPAR polypeptide levels are detected via Western Blotting, flow cytometry, Enzyme-linked immunosorbent assay (ELISA), immunoprecipitation, immunoelectrophoresis, immunostaining, isoelectric focusing, High-performance liquid chromatography (HPLC), or mass-spectrometry.

In any of the preceding embodiments of the methods disclosed herein, the test biological sample is mucus, saliva, bronchial alveolar lavage (BAL), bronchial wash (BW), whole blood, cerebrospinal fluid (CSF), urine, plasma, serum, lymph, semen, synovial fluid, tears, amniotic fluid, bile, aqueous humor, or a bodily fluid.

In yet another aspect, the present disclosure provides a method for selecting patients affected by a senescence-associated pathology for treatment with senolytic CAR T cell therapy comprising: (a) detecting uPAR and/or soluble uPAR (suPAR) polypeptide levels in biological samples obtained from the patients; (b) identifying patients that exhibit uPAR and/or soluble uPAR (suPAR) polypeptide levels that are elevated by at least 5% compared to a predetermined threshold; and (c) administering an engineered immune cell that specifically targets uPAR to the patients of step (b). The senescence-associated pathology may be cancer, lung fibrosis, atherosclerosis, Alzheimer's disease, diabetes, osteoarthritis, liver fibrosis, or chronic kidney disease. In some embodiments, the engineered immune cell that specifically targets uPAR is any engineered immune cell disclosed herein. Additionally or alternatively, in some embodiments, the uPAR and/or suPAR polypeptide levels are detected via Western Blotting, flow cytometry, Enzyme-linked immunosorbent assay (ELISA), immunoprecipitation, immunoelectrophoresis, immunostaining, isoelectric focusing, High-performance liquid chromatography (HPLC), or mass-spectrometry. In any of the preceding embodiments of the methods disclosed herein, the biological samples comprise mucus, saliva, bronchial alveolar lavage (BAL), bronchial wash (BW), whole blood, cerebrospinal fluid (CSF), urine, plasma, serum, lymph, semen, synovial fluid, tears, amniotic fluid, bile, aqueous humor, or bodily fluids.

### Kits

In one aspect, the kits of the present technology comprise a therapeutic or prophylactic composition including an effective amount of any of the engineered immune cells disclosed herein in unit dosage form. In some embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the engineered immune cell can be provided together with instructions for administering the engineered immune cell to a subject having or at risk of developing cancer or a senescence-associated pathology, such as lung fibrosis, atherosclerosis, Alzheimer's disease, diabetes, liver fibrosis, chronic kidney disease, aging, or osteoarthritis. The instructions will generally include information about the use of the composition for the treatment or prevention of cancer or a senescence-associated pathology. In other embodiments, the instructions include at least one of the following: description of the therapeutic agent; dosage schedule and administration for treatment or prevention of cancer or a senescence-associated pathology or symptoms thereof; precautions; warnings; indications; counter-indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

In some embodiments, the at least one engineered immune cell of the present technology binds to target cells that express uPAR on the cell surface. The at least one engineered immune cell of the present technology may be provided in the form of a prefilled syringe or autoinjection pen containing a sterile, liquid formulation or lyophilized preparation (e.g., Kivitz et al., Clin. Ther. 28:1619-29 (2006)).

A device capable of delivering the kit components through an administrative route may be included. Examples of such devices include syringes (for parenteral administration) or inhalation devices.

The kit components may be packaged together or separated into two or more containers. In some embodiments, the containers may be vials that contain sterile, lyophilized formulations of engineered immune cell composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers.

In another aspect, the present disclosure provides kits comprising reagents for detecting uPAR/suPAR expression levels in a biological sample obtained from a subject, and instructions for detecting the presence of senescent cells (e.g., SASP) in the sample. Suitable reagents for detecting uPAR/suPAR expression levels are known in the art, and include those used in via Western Blotting, flow cytometry, Enzyme-linked immunosorbent assay (ELISA), immunoprecipitation, immunoelectrophoresis, immunostaining, isoelectric focusing, High-performance liquid chromatography (HPLC), or mass-spectrometry. uPAR/suPAR expression levels may assessed using uPAR-specific immunoglobulin compositions known in the art, as well as those described herein.

### EXAMPLES

### Example 1: General Experimental Methods

RNA Extraction, RNA-Seq Library Preparation and Sequencing: Total RNA was Isolated from:

1) Kras^{G12D}; p53^{-/-} cells after 8 days of treatment with vehicle (DMSO) or combined trametinib (25 nM) and palbociclib (500 nM);

2) Oncogene-induced senescent hepatocytes generated in C57BL/6 mice via hydrodynamic tail vein injection (HTVI). For each mouse, 25 μg of pT3-Caggs-Nras^{G12V}-IRES-GFP plasmids (or pT3-Caggs-Nras^{G12V;D38A}-IRES-GFP plasmids as control) and 5 μg CMV-SB13 were suspended in saline solution at the volume of 10% of the animal's body weight for administration. Six days after HTVI, mice were anesthetized and placed on the platform for liver perfusion. Sequential perfusions of HBSS containing EGTA and HBSS containing Collagenase IV were performed, followed by passing the dissociated liver cells through a 100 μM cell strainer. The hepatocytes were further washed by low glucose DMEM and low speed centrifugation. DAPI-negative/GFP-positive hepatocytes, indicating successful transduction of mutant NRas expression, were isolated through low pressure fluorescence-activated cell sorting.

3) The datasets from senescent or proliferating hepatic stellate cells were obtained from a previous study. Lujambio et al. Cell 153: 681 449-460 (2013). RNA-seq libraries were prepared from total RNA. After RiboGreen quantification and quality control by Agilent BioAnalyzer, 100-500 ng of total RNA underwent polyA selection and TruSeq library preparation according to instructions provided by Illumina (TruSeq Stranded mRNA LT Kit, RS-122-2102), with 8 cycles of PCR. Samples were barcoded and run on a HiSeq 4000 or HiSeq 2500 in a 50 bp/50 bp paired end run, using the HiSeq 3000/4000 SBS Kit or TruSeq SBS Kit v4 (Illumina).

RNA-seq read mapping, differential expression analysis and heatmap visualization: Resulting RNA-Seq data was analyzed by removing adaptor sequences using Trimmomatic. Bolger et al., Bioinformatics 30: 2114-2120 (2014). RNA-Seq reads were then aligned to GRCm38.91 (mm10) with STAR⁵⁰ and transcript count was quantified using featureCounts (Liao et al., Bioinformatics 30: 730 923-930 (2014)) to generate raw count matrix. Differential gene expression analysis and adjustment for multiple comparisons were performed using DESeq2 package (Love et al., Genome Biol 15: 550 (2014)) between experimental conditions, using two independent biological replicates per condition, implemented in R. Differentially expressed genes (DEGs) were determined by >2-fold change in gene expression with adjusted P-value<0.05. For heatmap visualization of DEGs, samples were z-score normalized and plotted using pheatmap package in R.

Functional annotations of gene clusters: Pathway enrichment analysis was performed in the resulting gene clusters with the Reactome database using enrichR. Chen et al., BMC Bioinformatics 14: 128 (2013). Significance of the tests was assessed using combined score, described as c=log(p)*z, where c is the combined score, p is Fisher exact test p-value, and z is z-score for deviation from expected rank.

Cell lines and compounds. The following cell lines were used in this study: murine KRAS^{G12D/+}; Trp53^{-/-} (KP) lung cancer cells (expressing luciferase (Luc)-green fluorescent protein (GFP); Ruscetti et al., Science 362: 1416-1422 (2018)), NALM6 and Eμ-ALL01 cells expressing firefly luciferase (FFLuc)-GFP). Davila et al., PLoS One 8: e61338 (2013); Feucht et al., Nat Med 25: 82-88 (2019). Cells were maintained in a humidified incubator at 37° C. with 5% CO₂. KP cells were grown in DMEM supplemented with 10% FBS and 100 IU/ml penicillin/streptomycin. NALM6 and Eμ-ALL01 cells were grown in complete medium composed of RPMI supplemented with 10% FBS, 1% L-glutamine, 1% MEM non-essential amino acids, 1% HEPES buffer, 1% sodium pyruvate, 0.1% beta-mercaptoethanol and 100 UI/ml penicillin/streptomycin. Human primary melanocytes were grown in dermal cell basal medium (ATCC, 200-030) supplemented with the adult melanocyte growth kit (ATCC, 200-042), 10% FBS and 100 IU/ml penicillin/streptomycin. All cell lines used were negative for mycoplasma.

For drug-induced senescence experiments in vitro, trametinib (S2673) and palbociclib (S1116) were purchased from Selleck Chemicals and dissolved in DMSO to yield 10 mM stock solutions, which were stored at −80° C. Ruscetti et al., Science 362: 1416-1422 (2018). Growth medium was changed every 2 days. For in vivo experiments trametinib was dissolved in a 5% hydroxypropyl methylcellulose and 2% Tween-80 solution (Sigma) and palbociclib was dissolved in sodium lactate buffer (pH 4). Ruscetti et al., Science 362: 1416-1422 (2018). Cerulein was purchased from Bachem. For doxorubicin treatment in vivo, doxorubicin was purchased from Selleck Chemicals (Selleckchem, S1208) and dissolved in PBS.

Senescence-associated beta galactosidase (SA-β-gal) staining. SA-β-Gal staining was performed at pH 6.0 for human cells and tissue and at pH 5.5 for mouse cells and tissue as previously described. Ruscetti et al., Science 362: 1416-1422 (2018). Fresh frozen tissue sections or adherent cells plated in 6-well plates were fixed with 0.5% glutaraldehyde in PBS for 15 minutes, washed with PBS supplemented with 1 mM MgCl₂ and stained for 5-8 hours in PBS containing 1 mM MgCl₂, 1 mg/ml X-Gal, 5 mM potassium ferricyanide and 5 mM potassium ferrocyanide. Tissue sections were counterstained with eosin. Five high power fields per well/section were counted and averaged to quantify the percentage of SA-β-Gal⁺ cells.

qRT-PCR. Total RNA was isolated using the RNeasy Mini Kit (Qiagen, Hilden Germany) and complementary DNA (cDNA) was obtained using TaqMan reverse transcription reagents (Applied Biosystems, Foster City Calif.). Real-time PCR was performed in triplicates using SYBR green PCR master mix (Applied Biosystems, Foster City Calif.) on the ViiA 7 Real-Time PCR System (Invitrogen, Carlsbad Calif.). GAPDH or B-actin served as endogenous normalization controls for mouse and human samples.

Mice. Mice were maintained under specific pathogen-free conditions, and food and water were provided ad libitum. The following mice were used: C57BL/6J background and NOD-scid IL2Rg^{null} (NSG) mice (purchased from The Jackson Laboratory). Mice were used at 8-12 weeks of age (5-7 weeks old for the xenograft experiments) and were kept in group housing. Mice were randomly assigned to the experimental groups.

Transposon-mediated intrahepatic gene transfer. Transposon-mediated intrahepatic gene transfer was performed as previously described. Kang et al., Nature 479: 547-551 (2011). In short, 8-12 week-old C57BL/6J mice received a saline solution at a final volume of 10% of their body weight containing 30 μg of total DNA composed of a 5:1 molar ratio of transposon-encoding vector (containing either the sequence for Nras^{G12V} or the sequence for the GTPase dead form Nras^{G12V;D38A}) to transposase encoding vector (Sleeping Beauty 13) through hydrodynamic tail vein injection (HTVI). For CAR T cell studies, NSG mice were intravenously injected with 0.5×10⁶ human CAR⁺ T cells or untransduced T cells 10 days after HTVI and monitored by bioluminescence imaging. At day 15 post CAR injection, mice were euthanized, and livers were removed and further analyzed.

Generation of murine Pancreatic Intraepithelial Neoplasias (PanIn). The mouse strains have been previously described. Livshits et al, eLife 7:e35216 (2018). To induce PanIn generation, KC;RIK (p48-Cre;RIK;LSLKrasG12D) male mice were treated with 8-hourly intraperitoneal injections of 80 μg/kg caerulein (Bachem) for two consecutive days. Mice were then euthanized 21 weeks later and their pancreata used for further analysis. Age matched C;RIK mice injected with PBS were used as control of normal pancreata.

In vivo induction of liver fibrosis. C57BL/6J mice were treated twice a week with 12 consecutive intraperitoneal (i.p.) injections of 1 ml/kg carbon tetrachloride (CCl₄) to induce liver fibrosis. For murine CAR T cell studies, cyclophosphamide (200 mg/kg) was administered 24 hours before T cell injection. Mice received 3×10⁶ CAR⁺ T cells or untransduced T cells and CCl₄ was continuously administered at the same dose and interval after T cell injection until day 20 post CAR injection. Animals were sacrificed 48-72 h after the last CCl₄ injection. NSG mice were treated twice a week with 8 consecutive i.p. injections of 1 ml/kg CCl₄ to induce liver fibrosis. For human CAR T cell studies, mice received 0.5×10⁶ CAR T cells or untransduced T cells and CCl₄ was continued once a week after CAR T injection until day 10 post CAR administration, when animals were sacrificed 48-72 h after the last CCl₄ injection. Venous blood was collected by facial vein puncture.

In vivo induction of doxorubicin-Induced senescence. 8-12 week-old female C57BL/6J mice were intraperitoneally injected with doxorubicin (Selleckchem, S1208) at either 5 mg/kg or 10 mg/kg body weight or with the same volume of PBS at the beginning of the experiment and 10 days later as described in Baar et al., Cell 169: 132-147 (2017). Venous blood was collected by facial vein puncture at days 10 and 20 after initial doxorubicin administration.

Patient-derived xenografts. Experiments with patient-derived xenografts were performed as described (Ruscetti et al., Science 362: 1416-1422 (2018)), using 5-7 week-old female NSG mice. MSK-LX27 graft was derived from a lung adenocarcinoma harboring KRAS^{G12D} and p53 mutations and a deletion in CDKN2A and was cut into pieces and inserted in the subcutaneous space. Mice were monitored daily, weighed twice weekly and caliper measurements begun when tumors became visible. Tumors were measured using the formula: tumor volume=(D×d²)/2 and when they reached a size of 100-200 mm³, mice were randomized based on starting tumor volume and treated with vehicle or trametinib (3 mg/kg body weight) and palbociclib (150 mg/kg body weight) per os for 4 consecutive days followed by 3 days off treatment. Experimental endpoints were achieved when tumors reached a size of 2000 mm³ or became ulcerated. Tumors were harvested at the experimental endpoint and tissue was divided evenly for 10% formalin fixation and OCT frozen blocks.

Patient samples. De-identified human samples from liver biopsies of patients with liver fibrosis from viral (HBV or HCV), alcoholic and non-alcoholic fatty liver disease were obtained. Human pancreatic intraepithelial neoplasia samples (PanINs) and human atherosclerosis samples were also obtained.

Histological analysis. Tissues were fixed overnight in 10% formalin, embedded in paraffin, and cut into 5 μm sections. Sections were subjected to hematoxylin and eosin staining, and to Sirius red staining for fibrosis detection. For fibrosis quantification, at least three whole sections from each animal were scanned and the images were quantified using NIH ImageJ software. The amount of fibrotic tissue was calculated relative to the total analyzed liver area as previously described. Lujambio et al., Cell 153: 449-460 (2013). Immunohistochemical and immunofluorescence stainings were performed following standard protocols. The following primary antibodies were used: human uPAR (R&D. AF807), mouse uPAR (R&D, AF534), NRAS (Santa Cruz, SC-31), SMA (abcam, Ab5694), mKATE (Evrogen, ab233), CD3 (abcam, ab5690), myc-tag (Cell Signaling, 2276), Ki-67 (abcam, ab16667), IL-6 (abcam, ab6672), Lba-1 (abcam, ab178846) and P-ERK^{T202/Y204} (Cell Signaling, 4370).

Flow cytometry. For analysis of uPAR expression in cell lines upon induction of senescence, KP cells were treated with trametinib (25 nM) and palbociclib (500 nM) or with vehicle (DMSO), and human primary melanocytes were continuously passaged for 15 passages and then trypsinized, resuspended in PBS supplemented with 2% FBS and stained with the following antibodies for 30 minutes on ice: PE-conjugated anti-mouse uPAR antibody (R&D. FAB531P) or APC-conjugated anti-human uPAR antibody (Thermo Fisher S.17-3879-42). The following fluorophore-conjugated antibodies were used for further characterization of T cells and target cells (‘h’ prefix denotes anti-human, ‘m’ prefix denotes anti-mouse): hCD45 APC-Cy7 (clone 2D1, BD, #557833), hCD4 BUV395 (clone 465 SK3, BD, #563550), hCD4 BV480 (clone SK3, BD, #566104), hCD62L BV421 (clone 466 DREG-56, BD, #563862), hCD62L BV480 (clone DREG-56, BD, #566174), hCD45RA 467 BV650 (clone HI100, BD, #563963), hCD25 BV650 (clone BC96, Biolegend, #302634) hPD1 BV480 (clone EH12.1, BD, #566112), hCD19 BUV737 (clone SJ25C1, BD, #564303), hCD271 PE (clone C40-1457, BD, #557196), hCD69 APC (clone FN50, Biolegend, #310910), hIL2 PE-Cy7 (clone MQ1-17H12, Invitrogen, #25-7029-42), hTNFa BV650 (clone Mab11, BD, #563418), hIFNg BUV395 (clone B27, BD, #563563), hTIM3 BV785 (clone F38-2E2, Biolegend, #345032), hCD8 PE-Cy7 (clone SK1, eBioscience, #25-0087-42), hCD8 APC-Cy7 (clone SK1, BD, #557834), hCD223 PerCP-eFluor710 (clone 3DS223H, eBioscience, #46-2239-42), hCD223 BV421 (clone 11C3C65, Biolegend, #369314), hGrB APC (clone GB12, Invitrogen, #MHGB05), hMyc-tag AF647 (clone 9B11, Cell Signaling Technology, #2233S), hCD19 PB (clone SJ25-C1, Invitrogen, #MHCD1928), mCD19 PE (clone 1D3/CD19, Biolegend, #152408), hCD87 APC (clone VIM5, eBioscience, #17-3879-42), hCD87 PerCp-eFluor710 (clone VIM5, eBioscience, #46-3879-42), muPAR PE (R&D Systems, FAB531P), muPAR AF700 (R&D Systems, FAB531N), 7-AAD (BD, #559925), DAPI (Life technologies D1306) and LIVE/DEAD Fixable Violet (L34963, Invitrogen) were used as a viability dyes.

CAR staining was performed with Alexa Fluor 647 AffiniPure F(ab)2 Fragment Goat Anti-Rat IgG (Jackson ImmunoResearch, #112-6606-072). For cell counting, CountBright Absolute Counting Beads were added (Invitrogen) according to the manufacturer's instructions. For in vivo experiments, Fc receptors were blocked using FcR Blocking Reagent, mouse (Miltenyi Biotec). For intracellular cytokine secretion assay, cells were fixed and permeabilized using Cytofix/Cytoperm Fixation/Permeabilization Solution Kit (BD Biosciences) according to the manufacturer's instructions. Flow cytometry was performed on an LSRFortessa instrument (BD Biosciences) or Cytek Aurora (CYTEK) and data were analyzed using FlowJo (TreeStar).

For in vivo sample preparation, livers were dissociated using MACS Miltenyi Biotec liver dissociation kit (130-1-5-807), filtered through a 100 μm strainer, washed with PBS, and red blood cell lysis was achieved with an ACK (Ammonium-Chloride-Potassium) lysing buffer (Lonza). Cells were washed with PBS, resuspended in FACS buffer and used for subsequent analysis.

Detection of suPAR levels. suPAR levels from cell culture supernatant of murine plasma were evaluated by enzyme-linked immunosorbent assay (ELISA) according to the manufacturer's protocol (R&D systems, DY531 (mouse) or DY807 (human)).

Liverfunction tests. Serum alanine transaminase (ALT) and albumin levels in murine serum were measured according to the manufacturer's protocol, using the EALT-100 (ALT) and DIAG-250 (albumin) kits from BioAssay systems.

Isolation, expansion and transduction of human T cells. All blood samples were handled following the required ethical and safety procedures. Peripheral blood was obtained from healthy volunteers and buffy coats from anonymous healthy donors were purchased from the New York Blood Center. Peripheral blood mononuclear cells were isolated by density gradient centrifugation. T cells were purified using the human Pan T Cell Isolation Kit (Miltenyi Biotec), stimulated with CD3/CD28 T cell activator Dynabeads (Invitrogen) as described (Feucht et al., Nat Med 25: 82-88 (2019)) and cultured in X-VIVO 15 (Lonza) supplemented with 5% human serum (Gemini Bio-Products), 5 ng/ml interleukin-7 and 5 ng/ml interleukin-15 (PeproTech). T cells were enumerated using an automated cell counter (Nexcelom Bioscience).

48 hours after initiating T cell activation, T cells were transduced with retroviral supernatants by centrifugation on RetroNectin-coated plates (Takara). Transduction efficiencies were determined 4 days later by flow cytometry and CAR T cells were adoptively transferred into mice or used for in vitro experiments.

Isolation, expansion and transduction of mouse T cells. Mice were euthanized and spleens were harvested. Following tissue dissection and red blood lysis, primary mouse T cells were purified using the mouse Pan T cell Isolation Kit (Miltenyi Biotec). Purified T cells were cultured in RPMI-1640 (Invitrogen) supplemented with 10% fetal bovine serum (FBS; HyClone), 10 mM HEPES (Invitrogen), 2 mM L-glutamine (Invitrogen), MEM nonessential amino acids 1×(Invitrogen), 0.55 mM β-mercaptoethanol, 1 mM sodium pyruvate (Invitrogen), 100 IU/mL of recombinant human IL-2 (Proleukin; Novartis) and mouse anti-CD3/28 Dynabeads (Gibco) at a bead:cell ratio of 1:2. T cells were spinoculated with retroviral supernatant collected from Phoenix-ECO cells 24 hours after initial T cell expansion as described (Kuhn et al., Cancer Cell 35: 473-488 (2019)) and used for functional analysis 3-4 days later.

Genetic modification of T cells. The human and murine SFG γ-retroviral m.uPAR-28ζ plasmids were constructed by stepwise Gibson Assembly (New England BioLabs) using the SFG-1928ζ backbone as previously described. Brentjens et al., Nat Med 9: 279-286 (2003); Davila et al., PLoS One 8: e61338 (2013); Maher et al., Nat Biotechnol 20: 70-75 (2002), Brentjens et al., Clin Cancer Res 13: 5426-5435 (2007); Hagani et al., J Gene Med 1: 341-351 (1999). The amino acid sequence for the single-chain variable fragment (scFv) specific for mouse uPAR was obtained from the heavy and light chain variable regions of a selective monoclonal antibody against mouse uPAR (R&D.MAB531-100) through Mass Spectometry performed by Bioinformatics Solutions, Inc. In the human SFG-m.uPAR-h28z CARs, the m.uPAR scFv is thus preceded by a human CD8a leader peptide and followed by CD28 hinge-transmembrane-intracellular regions, and CD3ζ intracellular domains linked to a P2A sequence to induce coexpression of truncated low-affinity nerve growth factor receptor (LNGFR). In the mouse SFG-m.uPAR-m28z CARs, the m.uPAR scFv is preceded by a murine CD8a leader peptide and followed by the Myc-tag sequence (EQKLISEEDL (SEQ ID NO: 58)), murine CD28 transmembrane and intracellular domain and murine CD3ζ intracellular domain. Kuhn et al., Cancer Cell 35: 473-488 (2019). Plasmids encoding the SFGγ retroviral vectors were used to transfect gpg29 fibroblasts (H29) in order to generate VSV-G pseudotyped retroviral supernatants, which were used to construct stable retroviral-producing cell lines as described. Brentjens et al., Nat Med 9: 279-286 (2003); Kuhn et al., Cancer Cell 35: 473-488 (2019).

Cytotoxicity assays. The cytotoxicity of CAR T cells was determined by standard luciferase-based assays or by calcein-AM based cytotoxicity assays. For Luciferase-based assays target cells expressing firefly luciferase (FFLuc-GFP) were co-cultured with CAR T cells in triplicates at the indicated effector:target ratios using black-walled 96 well plates with 5×10⁴ (for NALM6 and Eμ-ALL01) or 1.5×10⁴ (for KP) target cells in a total volume of 100 μl per well in RPMI or DMEM media, respectively. Target cells alone were plated at the same cell density to determine the maximal luciferase expression (relative light units (RLU)). 4 or 18 hours later, 100 μl luciferase substrate (Bright-Glo; Promega) was directly added to each well. Emitted light was detected in a luminescence plate reader. Lysis was determined as (1−(RLUsample)/(RLUmax))×100.

For calcein-AM based assays, target cells (NALM6) were loaded with 20 μM calcein-AM (Thermo Fisher Scientific) for 30 minutes at 37° C., washed twice, and co-incubated with CAR T cells in triplicates at the indicated effector:target ratios in 96 well-round-bottomed plates with 5×10³ target cells in a total volume of 200 μl per well in complete medium. Target cells alone were plated at the same cell density to determine spontaneous release, and maximum release was determined by incubating the targets with 2% Triton-X100 (Sigma). After a 4-hours coculture, supernatants were harvested and free calcein was quantitated using a Spark plate reader (Tecan). Lysis was calculated as: ((experimental release−spontaneous release)/(maximum release−spontaneous release))×100

Statistical analysis. Data are presented as means±s.e.m. or means±s.d. Statistical analysis was performed by Student's t-test using GraphPad Prism 6.0 (GraphPad Software). P-values<0.05 were considered to be statistically significant. Survival was determined using the Kaplan-Meier method. No statistical method was used to predetermine sample size in animal studies. Animals were allocated at random to treatment groups.

NASH diet. Mice were started on NASH diet (TD.160785, Teklad) with fructose-containing drinking water (23.1 g of fructose and 18.9 g of glucose dissolved in 1 liter of water and then filter sterilized) at 8 weeks of age.

### Example 2: uPAR is a Cell Surface and Secreted Biomarker of Senescence

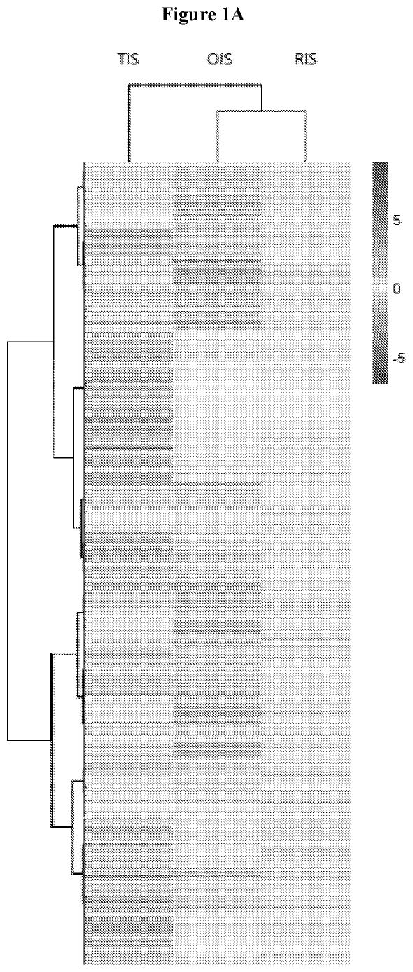

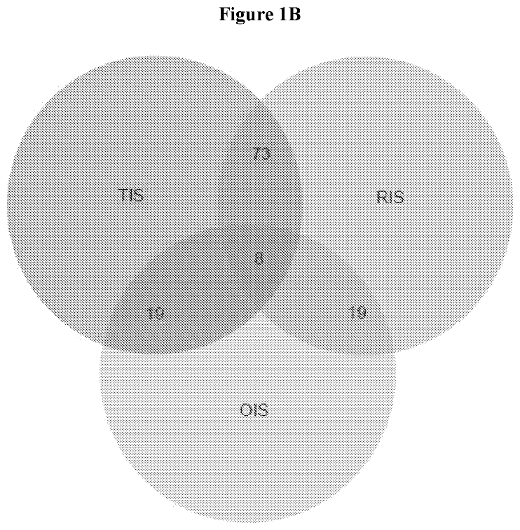

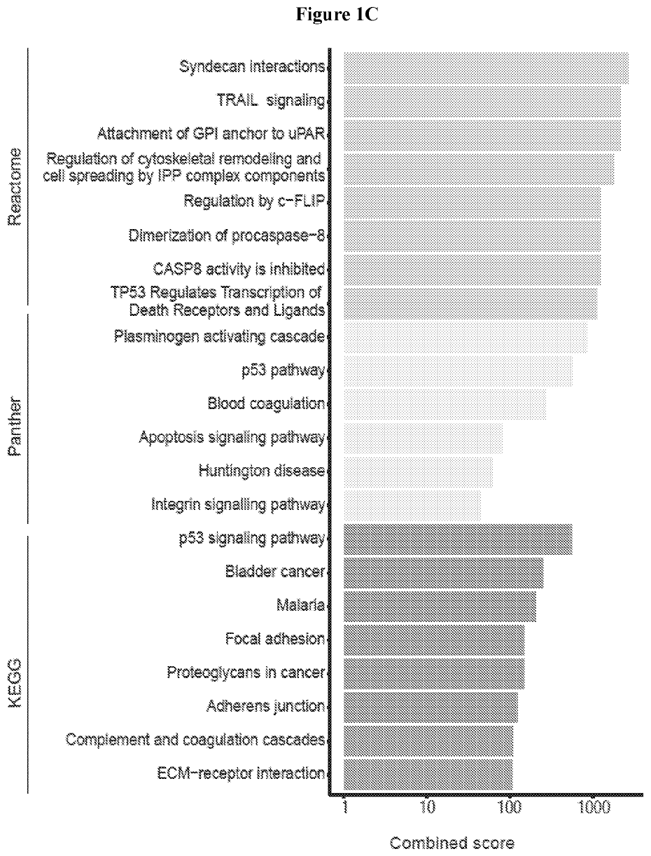

To identify cell surface proteins that are broadly and specifically upregulated in senescent cells, RNAseq datasets derived from the following three independent and robust models of senescence were compared: 1) Therapy-induced senescence (TIS) in murine lung adenocarcinoma Kras^{G12D};p53^{-/-} (KP) cells triggered to senesce by the combination of MEK and CDK4/6 inhibitors as previously described²⁷; 2) Oncogene-induced senescence (OIS) in murine hepatocytes mediated by in vivo delivery of NRAS^{G12D} through hydrodynamic tail vein injection (HTVI) (Kang et al., Nature 479: 547-551 (2011)); and 3) culture-induced senescence of hepatic stellate cells purified from murine livers (FIG. 1A). To focus the study on cell surface molecules, genes encoding membrane proteins were specifically selected, and only molecules defined to be located in the plasma membrane with a confidence score higher than 3 (range 0-5) as determined by UniProtKB were included. As shown in FIG. 1B, with these criteria, 8 genes encoding cell surface molecules which were commonly upregulated upon senescence induction among the three datasets were identified. These genes were linked to extracellular matrix remodeling and the coagulation cascade, as shown in FIG. 1C.

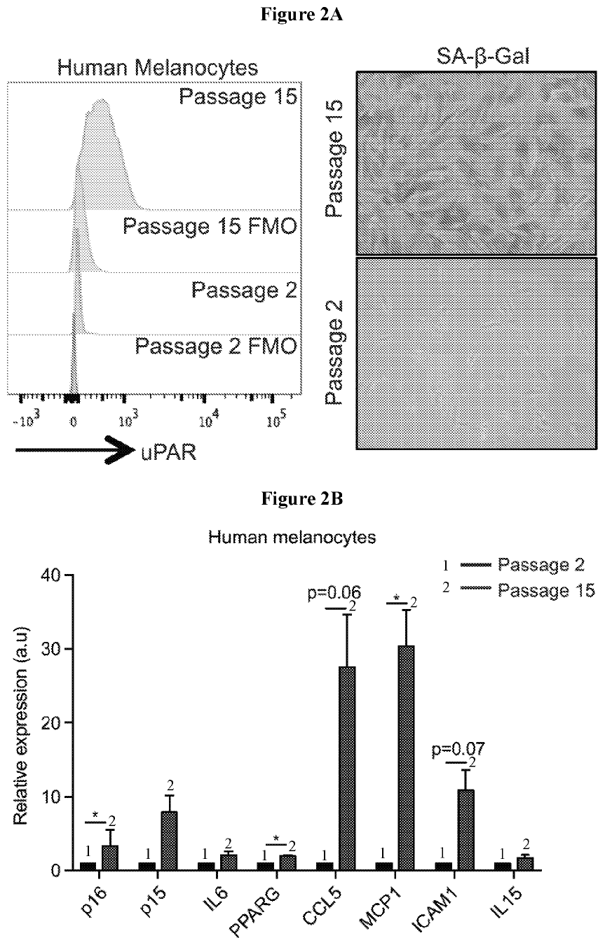

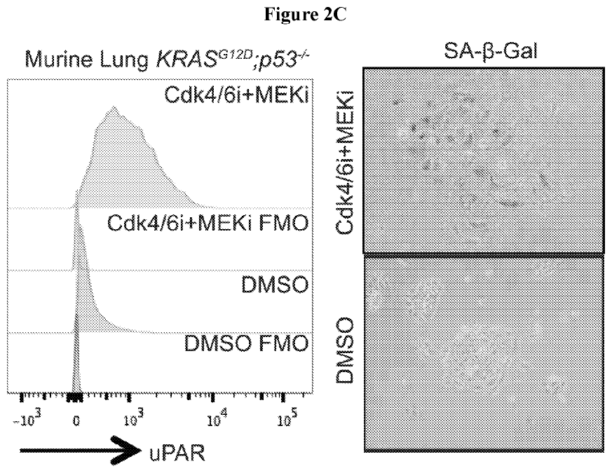

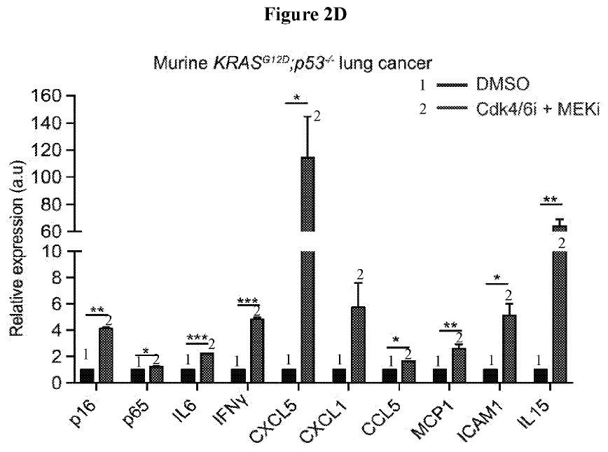

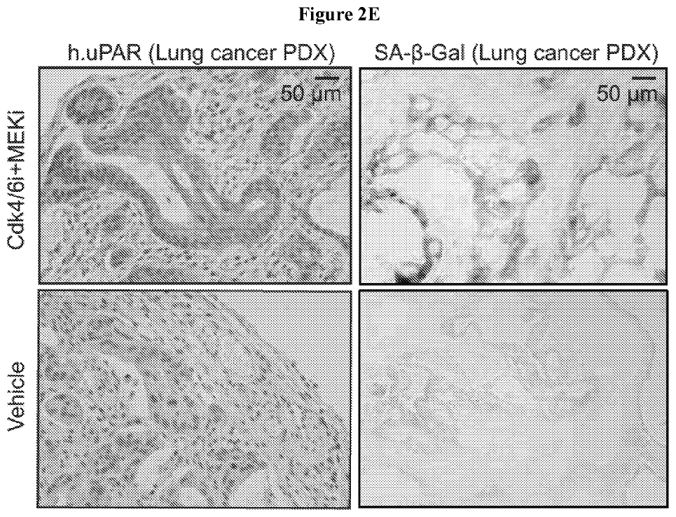

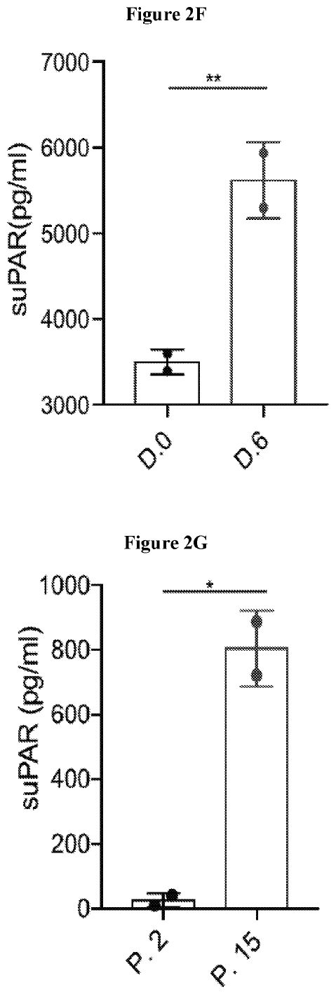

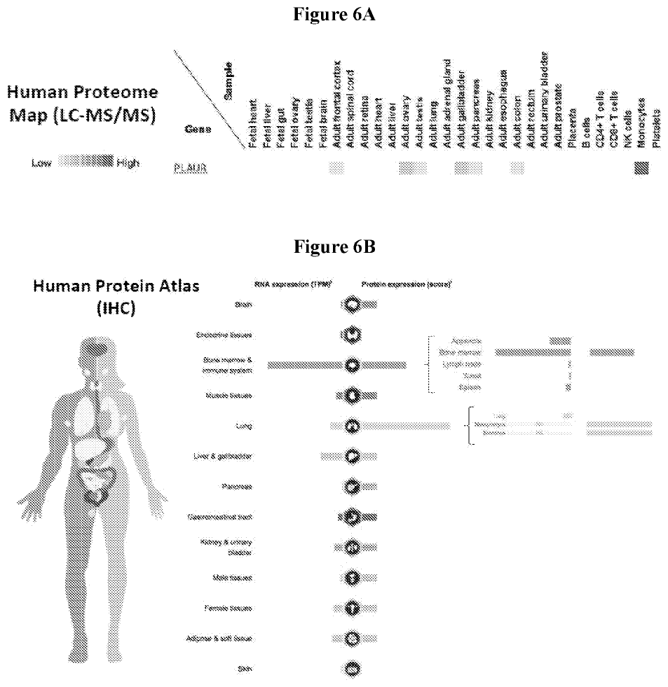

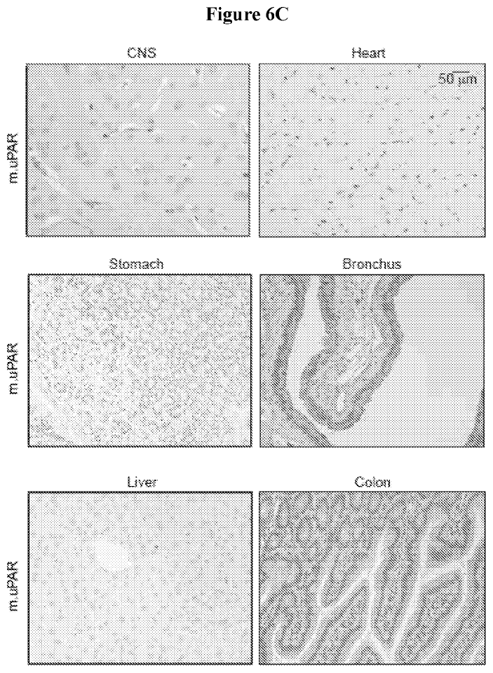

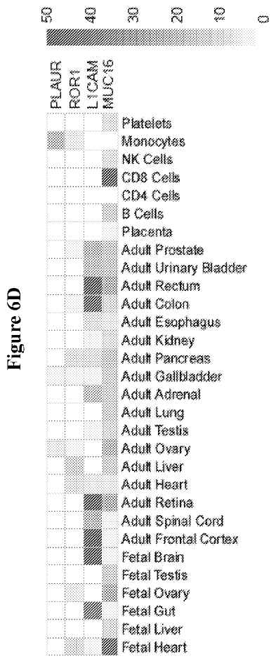

Given that ideal CAR targets should be expressed at high density on the target cells but not in vital tissues, these genes were ranked according to the magnitude of their upregulation (log 2 fold change) in senescent cells, and those highly expressed on vital tissues as determined by the Human Protein Atlas (HPA) and Human Proteome Map (HPM) were then excluded. Perna et al., Cancer Cell 32: 506-668 519 (2017). This selection process identified plaur, which encodes the urokinase plasminogen activator receptor (uPAR) as a candidate potentially suitable for CAR targeting. See FIGS. 2A, 2C, 2E, 3B, 3E, 5A-5B, 6D, and 9B. Accordingly, as shown in FIG. 6C, high uPAR expression could not be detected in vital tissues, including the central nervous system, heart, and liver. Low uPAR expression was observed in the respiratory epithelium of the bronchus, as shown in FIG. 6C. These findings are consistent with previous reports, that describe uPAR expression in the nasopharyngeal epithelium and on a subset of macrophages and neutrophilsi^{17,29}. Smith et al., Nat Rev Mol Cell Biol 11: 23-36 (2010); Simon et al., Blood 88: 3185-3194 (1996).

uPAR is the receptor for urokinase-type plasminogen activator (uPA), which upon binding to uPAR promotes the degradation of the extracellular matrix during fibrinolysis, wound healing or tumorigenesis. Smith et al., Nat Rev Mol Cell Biol 11: 23-36 (2010). Through interaction with other transmembrane receptors, uPAR also functions as a signaling receptor that promotes motility, invasion and survival of tumor cells and modulates neutrophil efferocytosis by macrophages. Smith et al., Nat Rev Mol Cell Biol 11: 23-36 (2010). Nonetheless, mice lacking uPAR are viable and fertile. Bugge et al., J Biol Chem 270: 16886-16894 (1995). In addition to its membrane-bound form, a portion of uPAR is proteolytically cleaved upon ligand binding, at either its glycosyl phosphatidylinositol (GPI) anchor or between the D1 and D2 domain of its three homologous domains, causing the secretion of soluble uPAR (suPAR).

To study whether uPAR was in fact broadly induced on senescent cells, its surface expression as well as suPAR levels in several established models of senescence were analyzed. In vitro, therapy-induced senescence was evaluated in KP lung cancer cells triggered to senesce by combined MEK and CDK4/6 inhibition (FIGS. 2C-2D) and replication-induced senescence in human primary melanocytes (FIGS. 2A and 2B). uPAR surface expression was examined by flow cytometry and suPAR was quantified by enzyme-linked immunosorbent assay (ELISA). As shown in FIGS. 2A and 2C, uPAR expression was markedly increased on senescent cells relative to non-senescent controls, and suPAR was considerably elevated in the senescent-cell supernatant in both models.

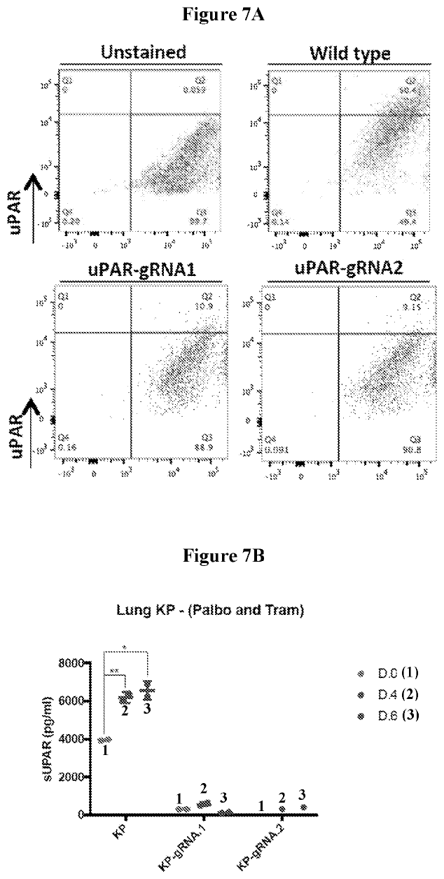

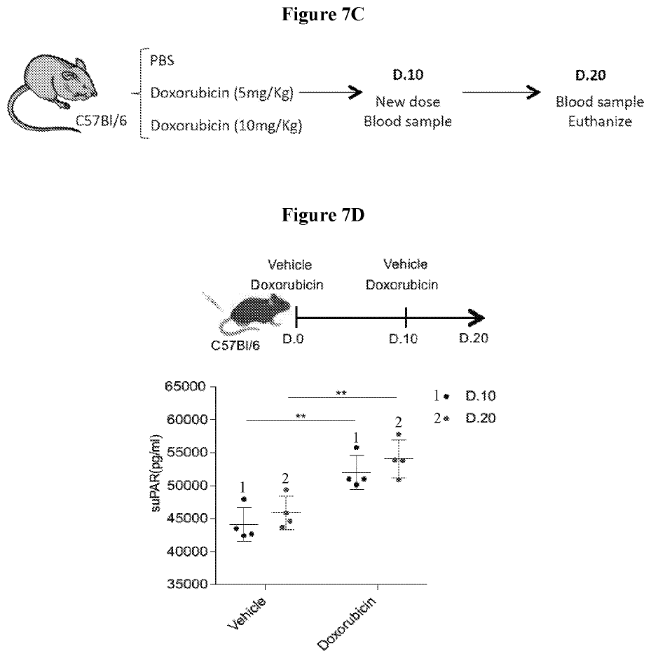

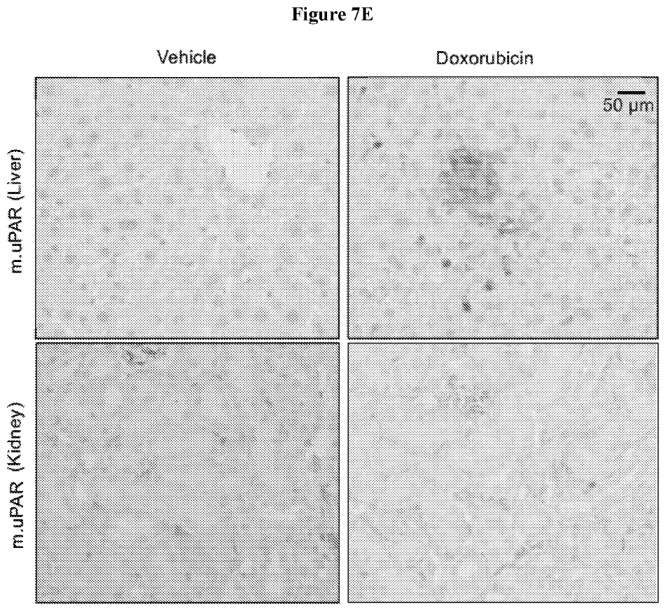

To determine whether uPAR is also upregulated upon senescence induction in vivo, uPAR expression was examined by immunohistochemistry and suPAR plasma levels were measured by ELISA in well-described mouse models of senescence. Thus, the following models were studied: a patient-derived xenograft (PDX) model of non-small cell lung cancer (NSCLC), in which mice were treated with combined MEK and CDK4/6 inhibitors (Ruscetti et al., Science 362: 1416-1422 (2018); See FIG. 2E)), and a model of chemotherapy-induced senescence in normal tissues (Baar et al., Cell 169: 132-147 (2017)), in which mice receive high dosages of the chemotherapeutic agent doxorubicin (FIGS. 7C, 7D-7E). Importantly, a time-dependent and dose-dependent correlation with the plasma levels of suPAR in the treated animals was observed. Both membrane-bound uPAR (FIG. 7A) and suPAR (FIG. 7B) were significantly upregulated over time in KP treated cells, whereas no upregulation was observed in uPAR KO KP cells.

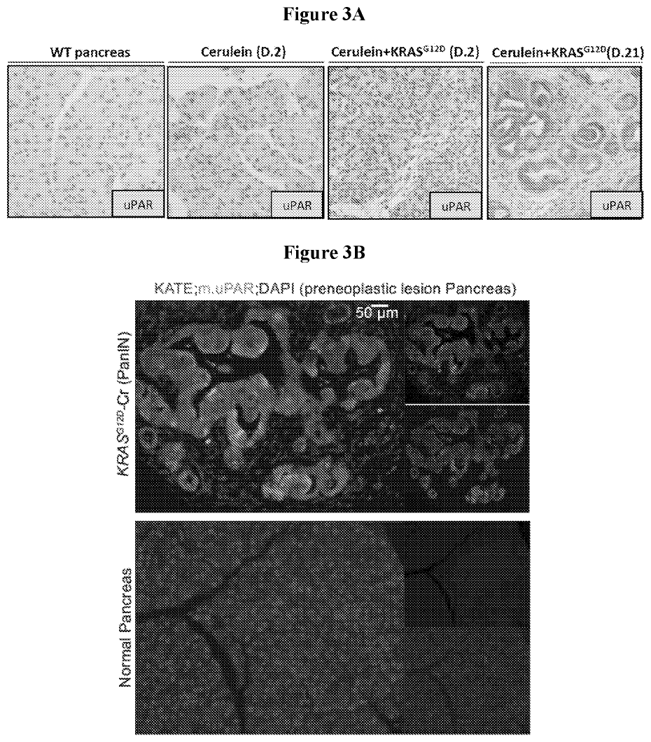

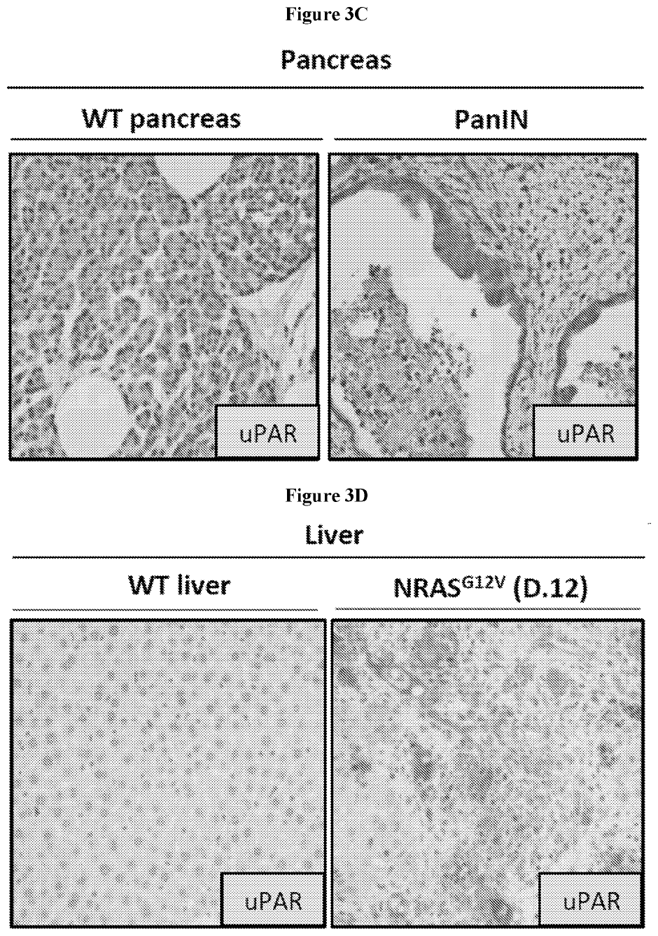

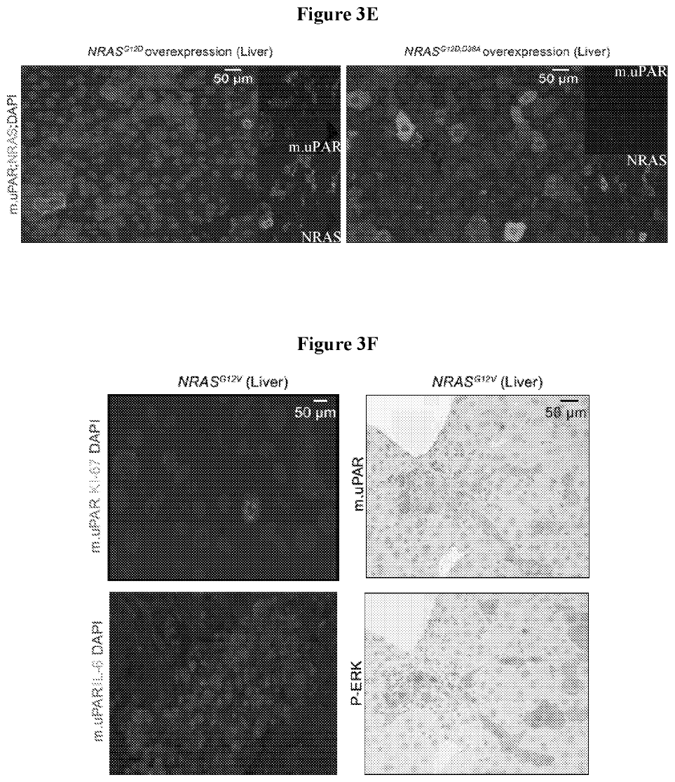

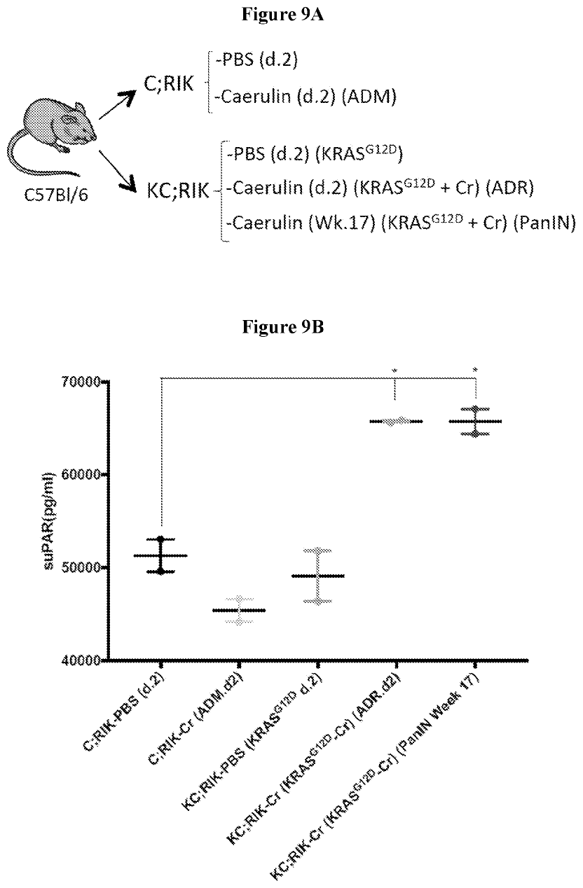

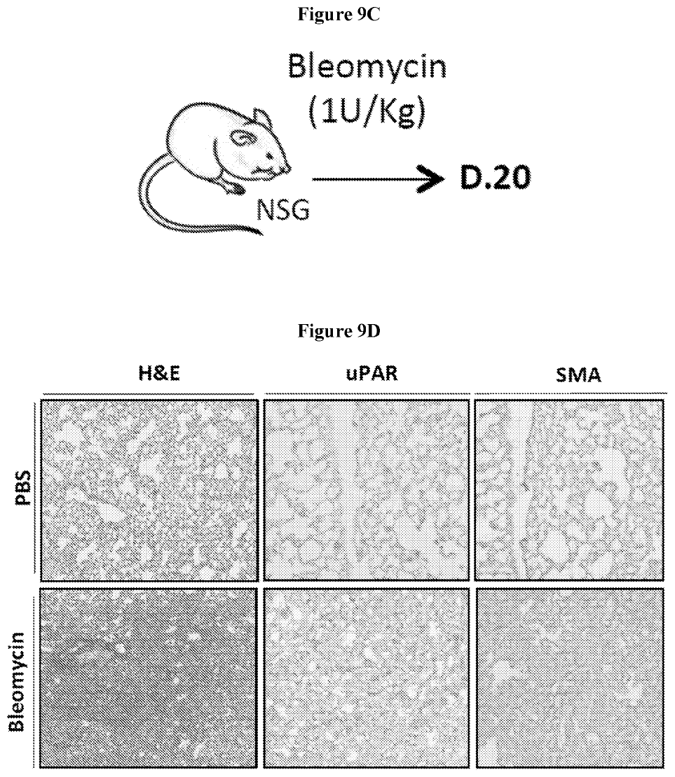

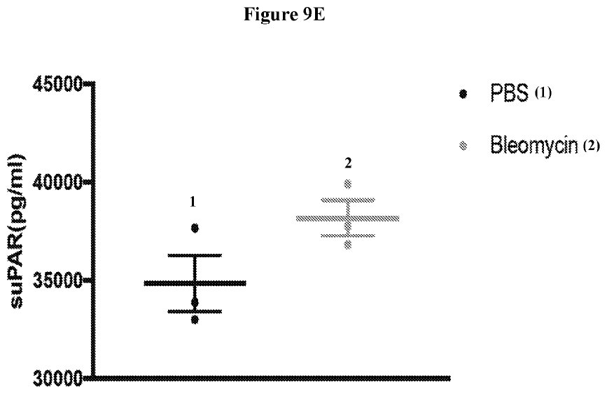

Additionally, two different models of oncogene-induced senescence triggered either by overexpression of Nras^{G12D} (but not the GTPase dead version Nras^{G12D;D38A}) through HTVI were examined (FIGS. 3E and 3F) or by endogenous Kras^{G12D} expression in a murine model of senescent pancreatic intraepithelial neoplasia (PanIN) (Saborowski et al., Genes Dev 28: 85-97 (2014); see FIGS. 3B and 9B). Surface uPAR was also present in vivo in murine senescent PanINs generated by chronic injury induced with cerulein and KRAS^{G12D} (FIG. 3A), and (iii) in hepatocytes induced to senescence by overexpression of NRAS^{G12D} through HTVI (FIG. 3D). Upregulation of uPAR was specific to senescent cells (FIG. 3A and FIG. 3C) as no expression was observed upon acute injury (FIG. 3A) and uPAR co-localized with specificity in the senescent cells.

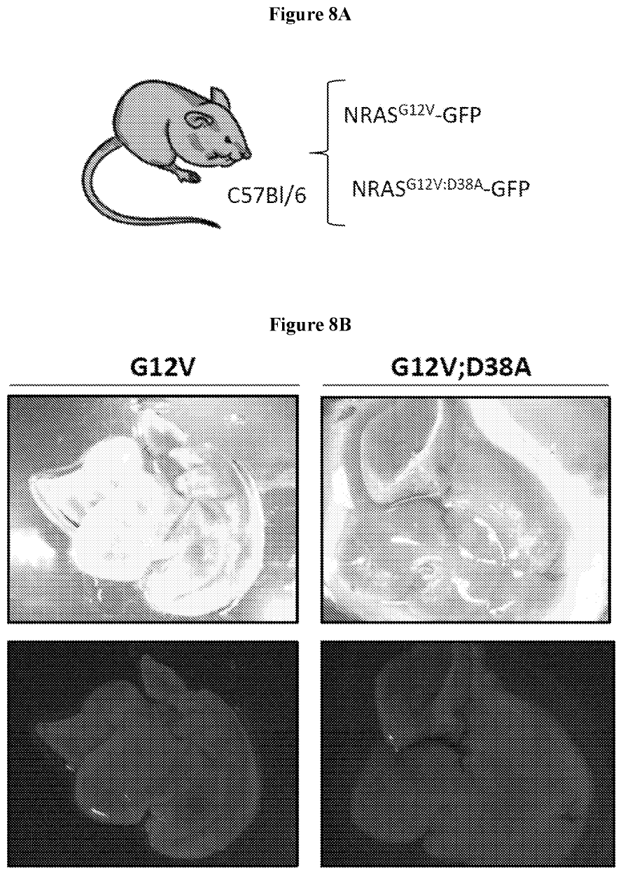

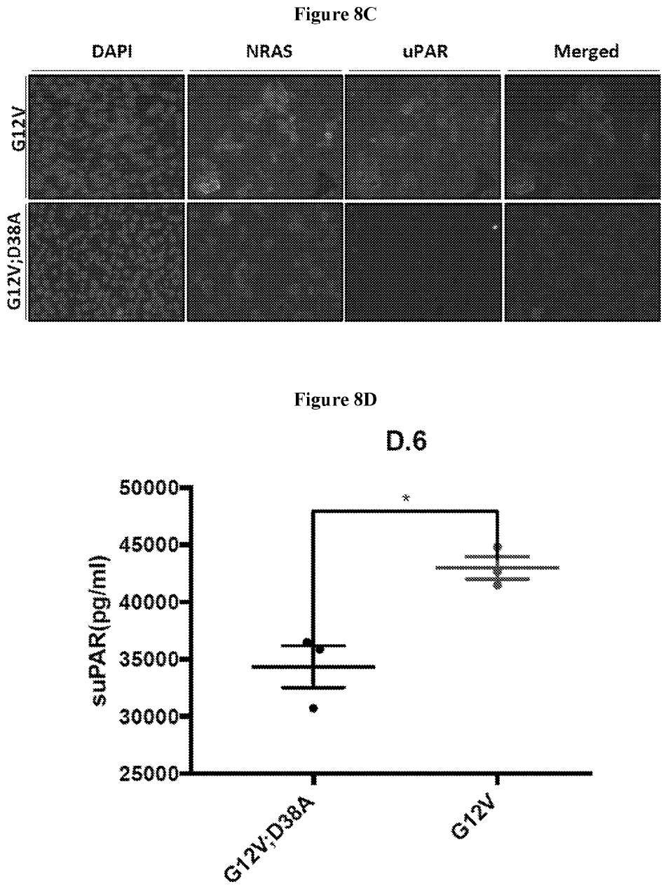

Further, a correlation between suPAR levels and senescence burden in an in vivo model of OIS in which NRAS^{G12D} or NRAS^{G12D; D38A} (a GTPase dead form) was overexpressed in murine hepatocytes by HTVI was also observed (FIGS. 8A and 8B). As described above, membrane uPAR expression was observed (which co-localized with NRAS expression) in the livers of NRAS^{G12D} treated mice (senescent hepatocytes) but not in those of NRAS^{G12D; D38A} treated mice (FIG. 8C). Importantly, significantly higher levels of suPAR were detected in the plasma from the former compared to the latter (FIG. 8D). Using a previously described mouse model to induce ADM, ADR and late PanINs (see Livshits, G. et al. eLife 7:e35216 (2018), and Saborowski, M. et al. Genes & Dev 28:85-97 (2014)) (FIG. 9A), a significant increase of suPAR was detected in the blood from mice with either ADR or PanIN, but not in ADM or in acute injury setting, highlighting the specificity of suPAR as a biomarker of senescence (FIG. 9B). Higher levels of suPAR were also detected in the plasma of mice with bleomycin-induced lung fibrosis (FIGS. 9C-9E), where senescent fibroblasts had been previously shown to contribute to disease (see Munoz-Espin, D. et al. EMBO Mol Med 10:e9355 (2018)).

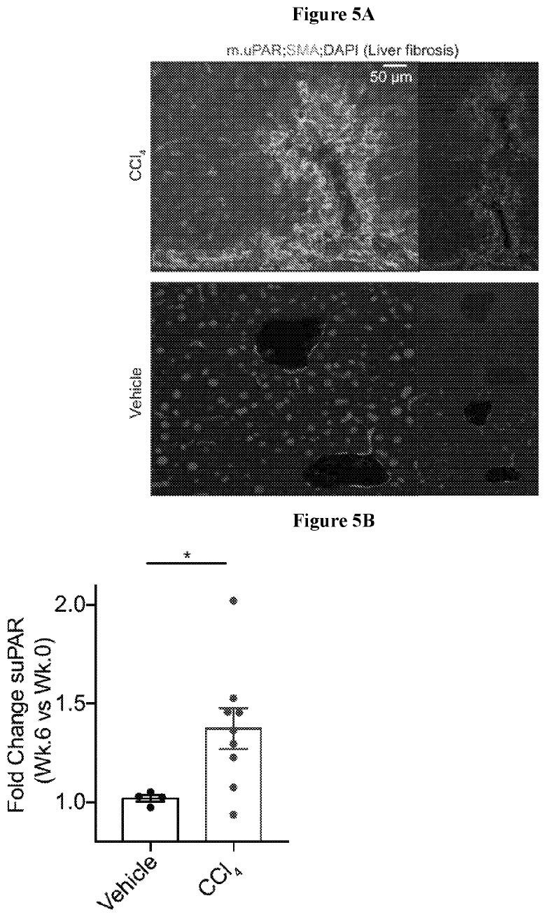

Finally, a mouse model of carbon tetrachloride (CCl₄)-induced liver fibrosis in which senescent hepatic stellate cells contribute to the pathophysiology was examined (FIGS. 5A-5B). Krizhanovsky et al., Cell 134: 657-667 (2008); Lujambio et al., Cell 153: 449-460 (2013). As shown in FIGS. 2E, 3B, 3E-3F, 5A-5B, 7D-7E and 9B, in each of these systems, the senescence-inducing treatment led to an increase in uPAR-positive cells and circulating suPAR compared to controls.

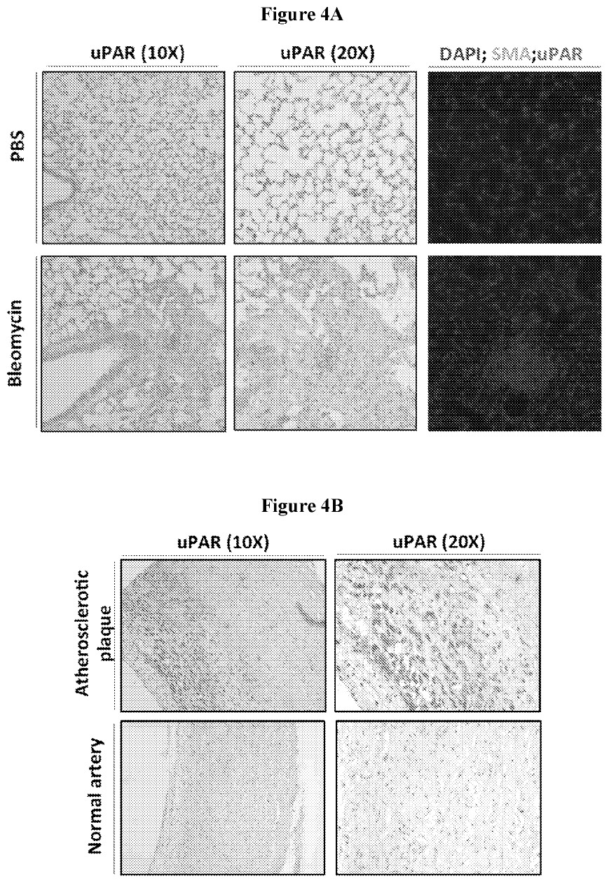

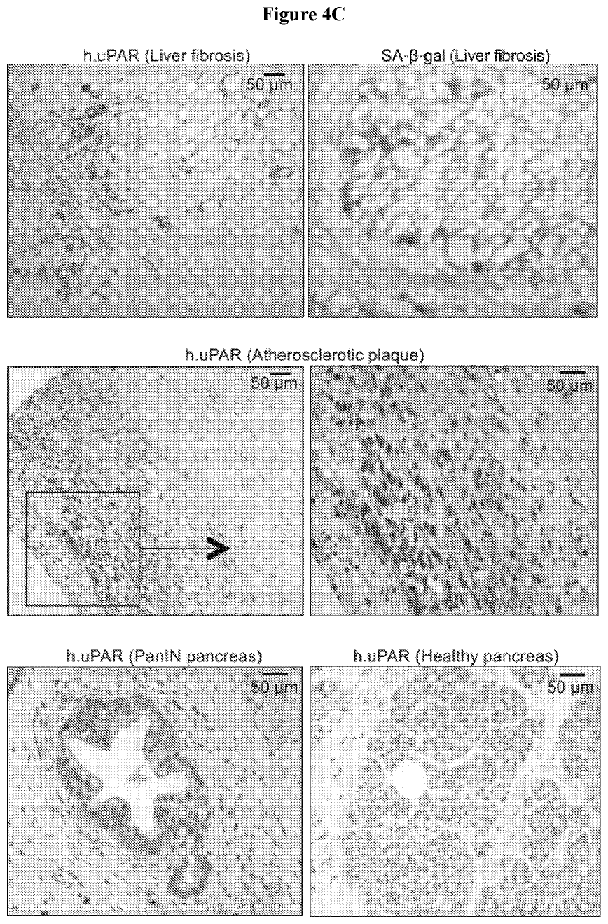

To determine whether uPAR is highly expressed in human tissues linked to senescence-associated pathologies, uPAR expression was analyzed in tissue samples from human patients with liver fibrosis arising from different etiologies (viral, alcoholic and nonalcoholic steatohepatitis). As shown in FIG. 4C (upper panel), high uPAR expression was detected in these samples and uPAR positive cells followed the same histological expression pattern as senescent cells (based on SA-β-gal staining). uPAR was also highly expressed in atherosclerotic plaques from human carotid endarterectomy specimens, in line with previous reports correlating disease severity with the abundance of senescent intimal foam cells, as shown in FIG. 4C (middle panel). Childs et al., Science 354: 472-477 (2016). Furthermore, as shown in FIG. 4C (lower panel), high uPAR expression was detected in human pancreatic intraepithelial neoplasia, but not in normal pancreas tissue. Besides the human senescence-associated pathologies shown herein, increased uPAR and or suPAR levels occur in patients with osteoarthritis, diabetes or idiopathic pulmonary fibrosis. Belcher et al., Ann Rheum Dis 55: 230-236 (1996); Guthoff et al., Sci Rep 7: 40627 (2017); Schuliga et al., Sci Rep 7: 41770 (2017). Together, these results strongly support the notion that uPAR is both a cell surface molecule commonly upregulated in senescence and is also a potential biomarker of senescent cell burden in the organism. uPAR was also upregulated in samples from previously reported senescence-associated diseases such as lung fibrosis (see Munoz-Espin, D. et al. EMBO Mol Med 10:e9355 (2018)) (FIG. 4A) or atherosclerosis (see Childs, B. G. et al. Science 354:472-477 (2016)) (FIG. 4B), while at the same time not being expressed in vital human and murine tissues by previously defined criteria (see Perna, F. et al. Cancer Cell 32:506-519 (2017)) (FIGS. 6A-6B).

As demonstrated herein, uPAR is upregulated in human liver fibrosis (induced by either hepatitis (HCV, HBV), or NASH or alcoholism) as well in human atherosclerosis and in human PanINs (FIGS. 3C, and 4B-4C). Additionally, human lung tumors (NSCLC) when induced to senesce upregulate uPAR expression and senescence (FIG. 2E).

Accordingly, the methods of the present technology are useful for detecting senescent cells in a biological sample obtained from a patient. The methods disclosed herein are also useful for selecting a patient suffering from a senescence-associated pathology for treatment with uPAR-specific CAR T cell therapy.

### Example 3: uPAR-CAR T Cells are Selectively Target uPAR Positive Target Cells

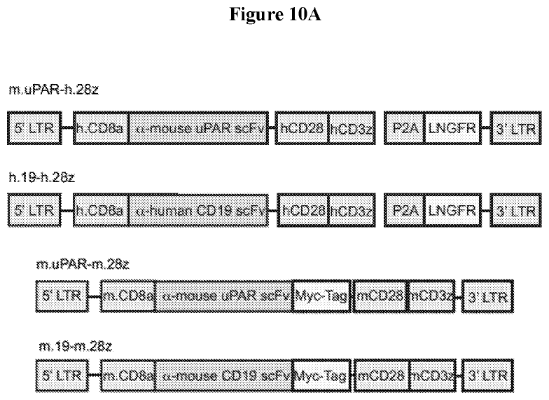

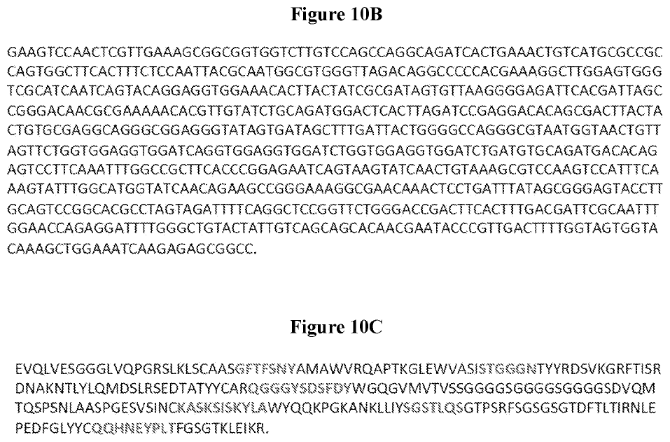

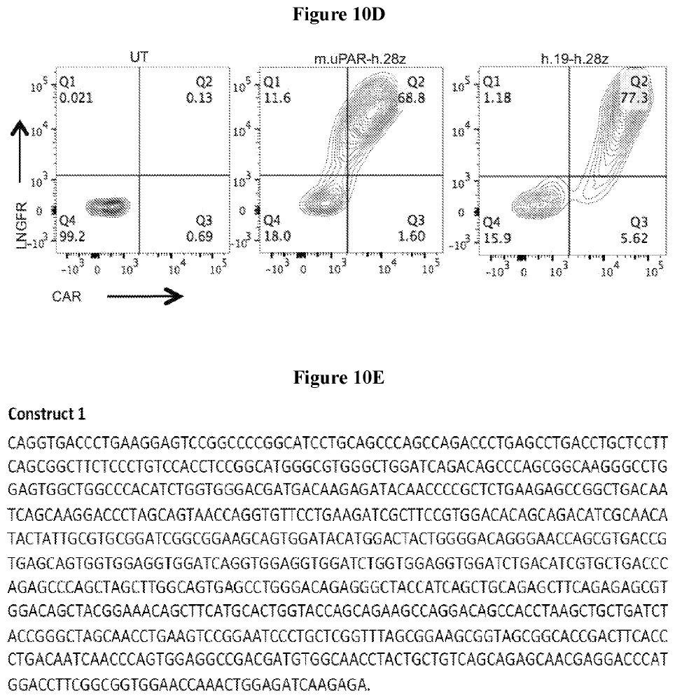

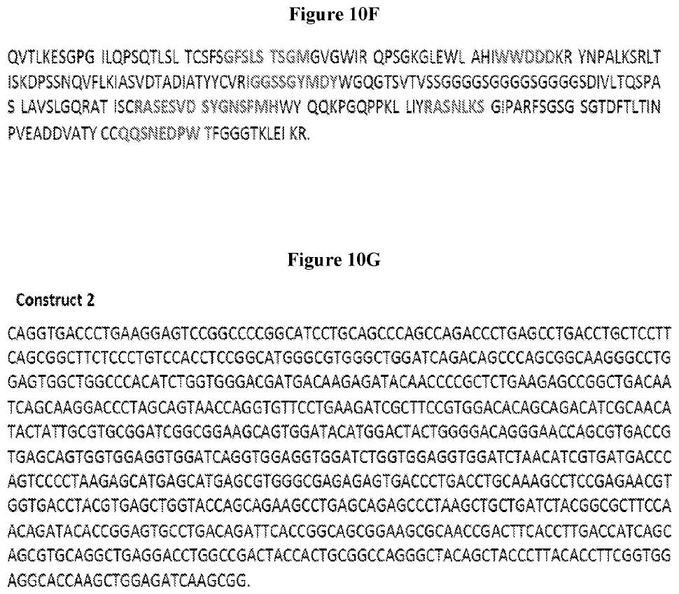

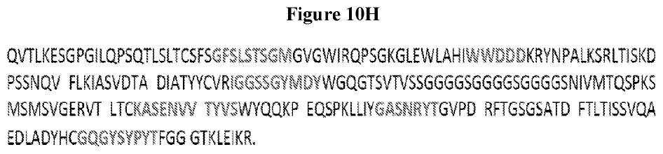

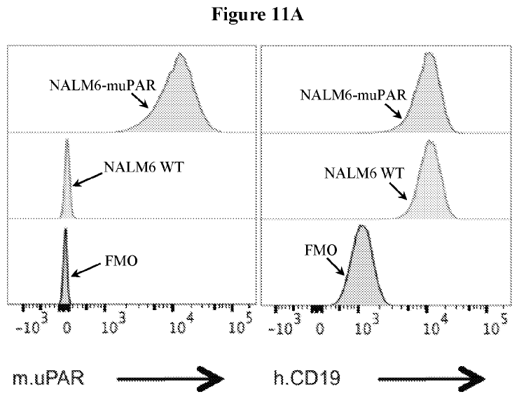

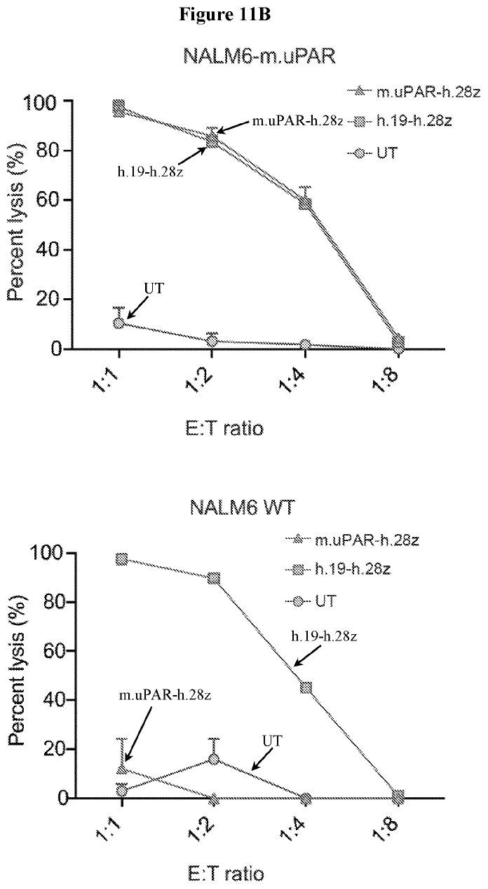

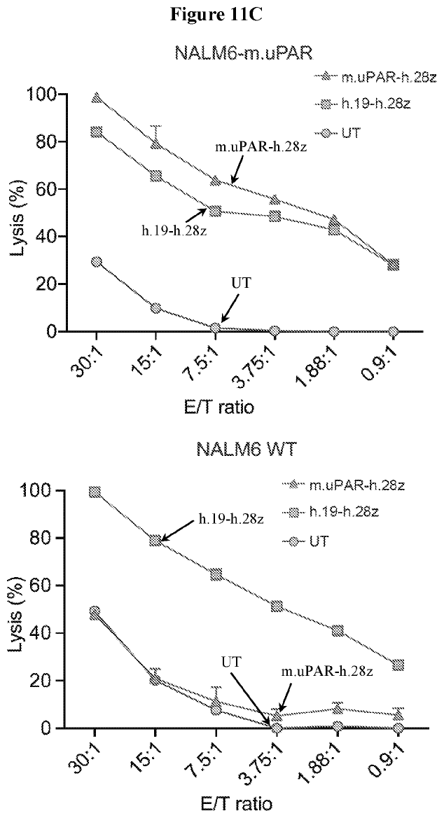

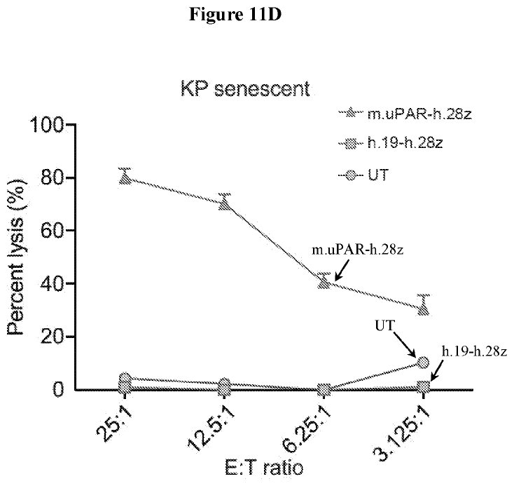

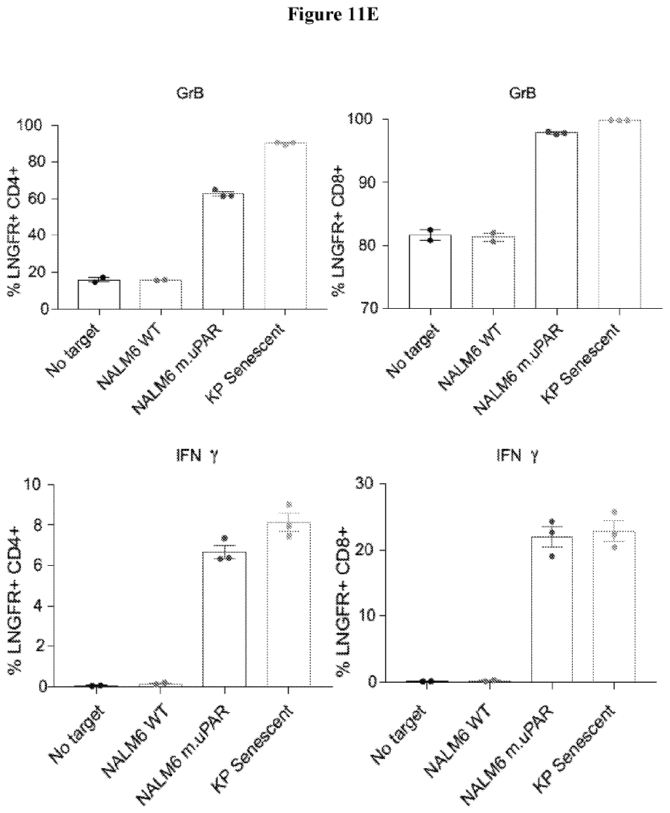

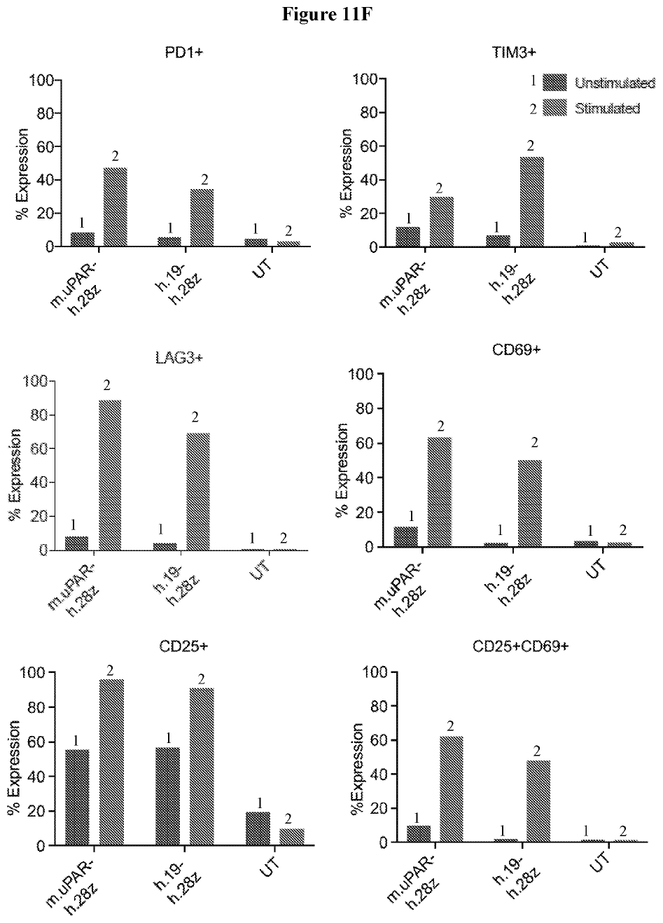

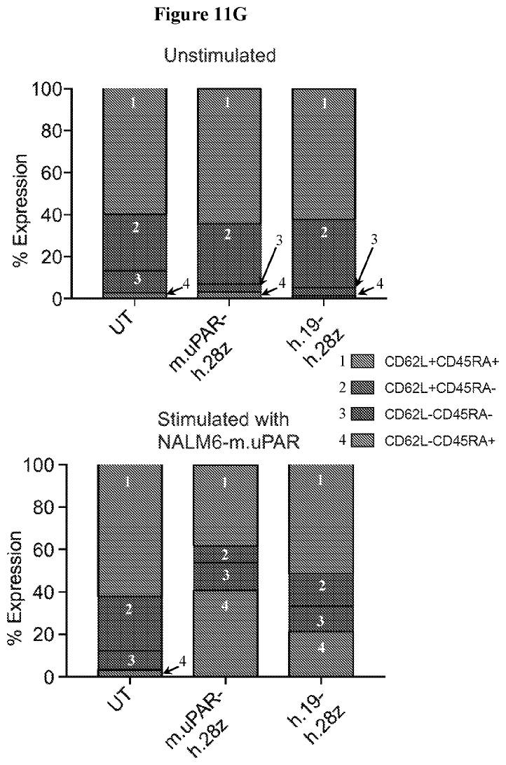

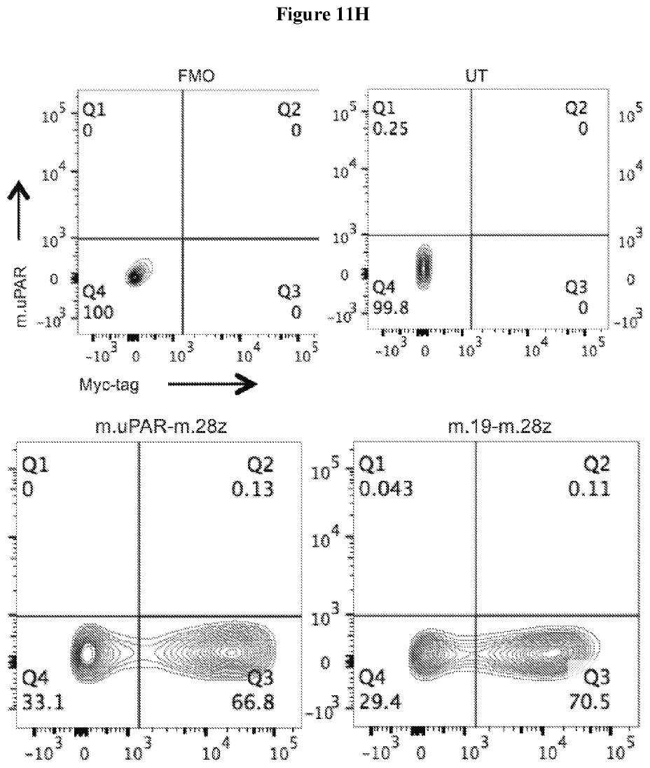

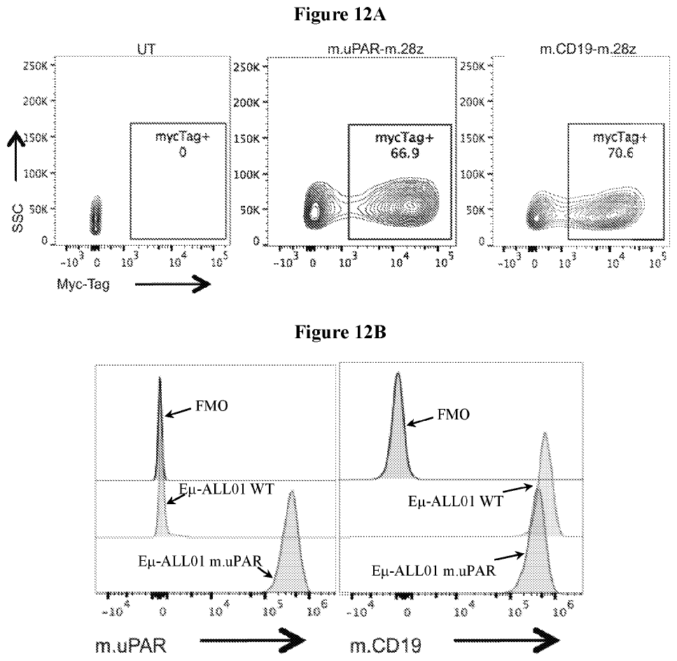

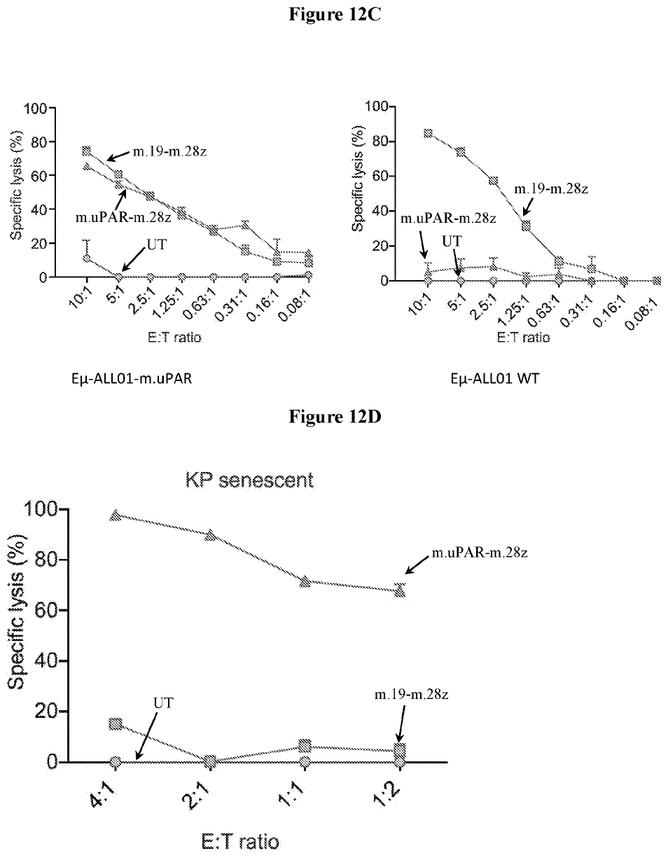

CAR T cells directed against murine and human uPAR were developed as an endogenous target of senescent cells (FIGS. 10B-10C and 10E-10H). The amino acid sequence of the heavy and the light chain of selective monoclonal antibodies was determined by mass spectrometry. Subsequently, the coding nucleotide sequence was derived from the amino acid sequence of each of the heavy and the light chain of selective monoclonal antibodies. Primary human T cells transduced with the SFG-mouse uPAR28ζ CAR construct effectively expressed the CAR in their plasma membrane. T cells were engineered to express a uPAR-specific CAR comprising an anti-murine or anti-human uPAR (m.uPAR) single chain variable fragment (scFv) linked to CD28 costimulatory and CD3ζ signaling domains (m.uPAR-28z) (See FIGS. 10A, 10D, 12A, and 21A-21B). To determine CAR activity in the well-characterized context of CD19 CARs (Brentjens et al., Nat Med 9: 279-286 (2003); Davila et al., PLoS One 8: e61338 (2013)), the human CD19⁺ pre-B acute lymphoblastic leukemia cell line (B-ALL) NALM6 and the mouse CD19⁺ B-ALL cell line Eμ-ALL01 were engineered to constitutively overexpress mouse uPAR and used them as models for CAR T cell targeting. As shown in FIGS. 11A and 12B, m.uPar-h.28z bound to target cells expressing mouse uPAR but not the control cells lacking mouse uPAR.

Accordingly, the methods disclosed herein are useful for detecting senescent cells in a biological sample obtained from a patient. The methods disclosed herein are also useful for selecting a patient suffering from a senescence-associated pathology for treatment with uPAR-specific CAR T cell therapy.

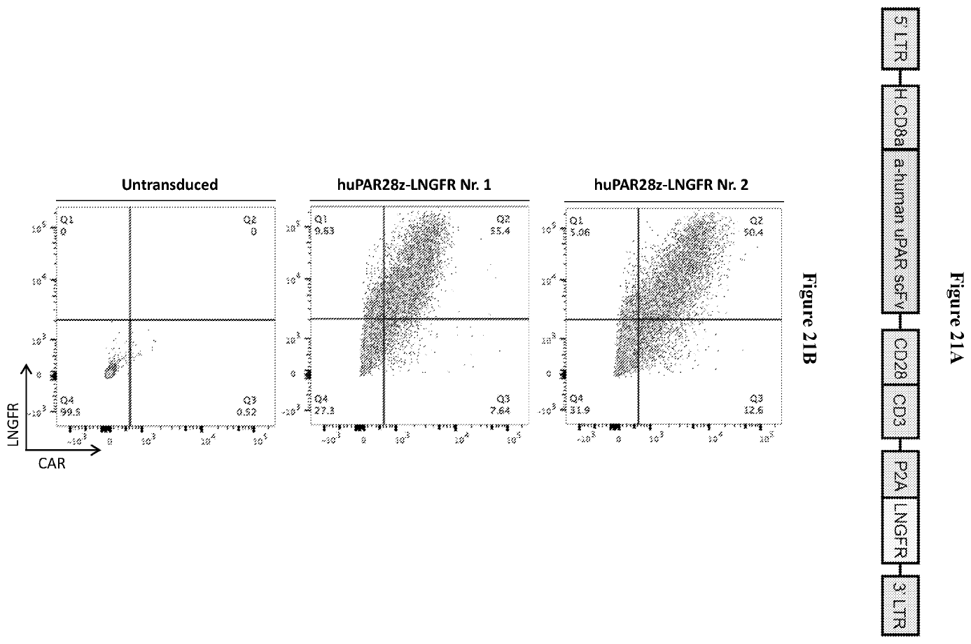

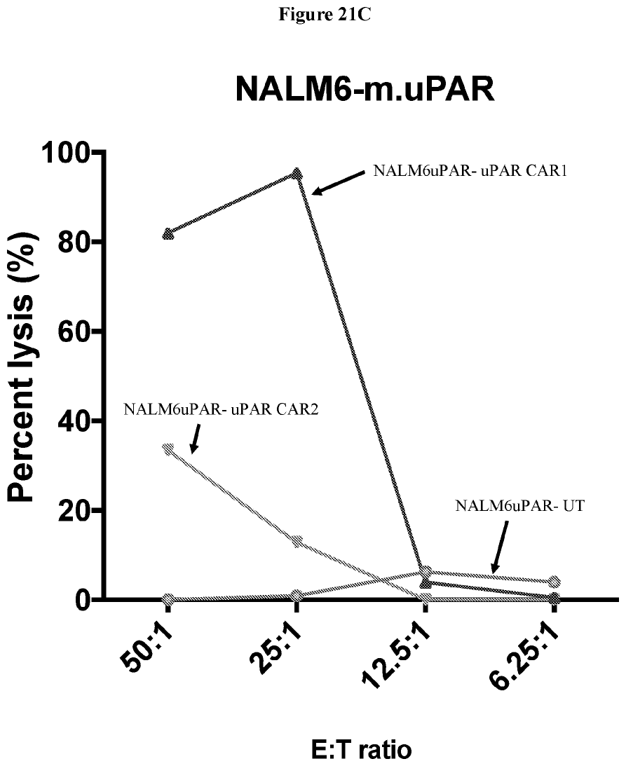

As shown in FIGS. 11B-11C, and 12C, retrovirally transduced human and mouse m.uPAR-28z CARs directed comparable in vitro cytotoxicity as their respective CD19 CAR (1928z or 0.19-h.28z) controls when targeting m.uPAR or endogenous CD19 in the same cell lines, while simultaneously sparing uPAR negative cells. Antigen-specific CAR activity was further confirmed by increased expression of T cell activation markers and enhanced T cell differentiation upon antigen stimulation, as shown in FIGS. 11F-11G. Importantly, as shown in FIGS. 11D, 11E and 12D, undiminished CAR functionality was evident against target cells expressing endogenous m.uPAR as demonstrated by high cytolytic activity and antigen-specific granzyme B (GrB) and IFNγ secretion of m.uPAR-28z CARs upon targeting senescent KP cells. Hence, both human and murine m.uPAR-28z CAR T cells selectively and efficiently targeted senescent cells in vitro. FIG. 21C also shows that h.uPAR-28z CARs were also effective in inducing in vitro cytotoxicity in uPAR positive cells compared to the untransduced negative controls.

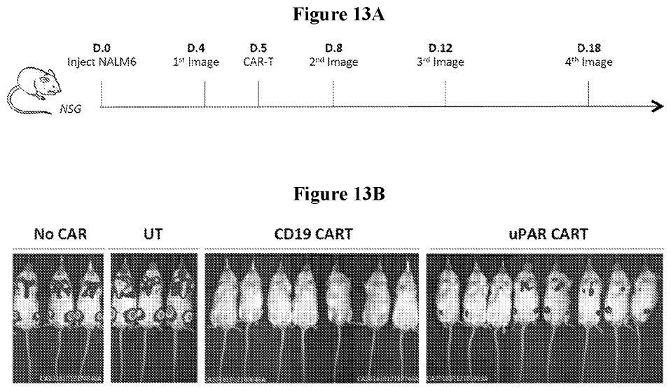

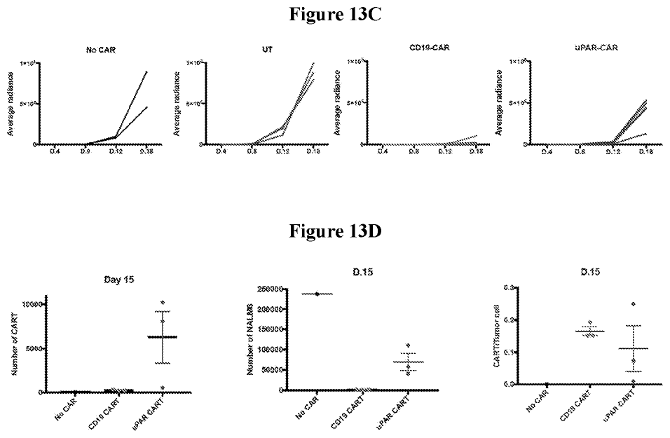

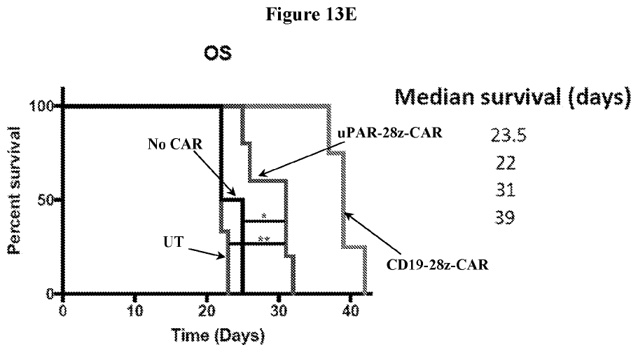

To determine whether the anti-mouse uPAR CAR T cells were also selective and effective in vivo, and to analyze potential toxicities of the anti-uPAR CAR T cells, uPAR-Nalm6 cells were injected into NSG mice and 5 days later infused either untransduced T cells, anti-human CD19 CAR T cells or anti-mouse uPAR CAR T cells (FIG. 13A). Tumor growth was significantly reduced in mice treated with anti-mouse uPAR CAR T cells compared to mice that received untransduced T cells or untreated; and this reduction was comparable to that observed in mice treated with anti-human CD19 CAR T cells (FIGS. 13B-13D). Thus, mice treated with the anti-uPAR CAR T cells demonstrated significantly increased survival compared to untreated mice or mice treated with untransduced T cells (FIG. 13E). Without wishing to be bound by theory, it is believed that it is likely that the tumor progression observed from day 12 onwards in the uPAR CAR T treated mice is due to down-regulation of uPAR expression in the tumor cells, a mechanism of resistance that has been previously shown in response to anti-CD19 CAR T cells and which remains to be investigated in the context of uPAR expression.

Accordingly, the CARs of the present technology are useful in the methods for treating or ameliorating the effects of a senescence-associated pathology in a subject in need thereof. Nonlimiting examples of senescence-associated pathologies include lung fibrosis, intraepithelial neoplasia, atherosclerosis, Alzheimer's disease, diabetes, liver fibrosis, chronic kidney disease, aging, or osteoarthritis.

### Example 4: uPAR-CAR T Cells are Selective Senolytics In Vivo

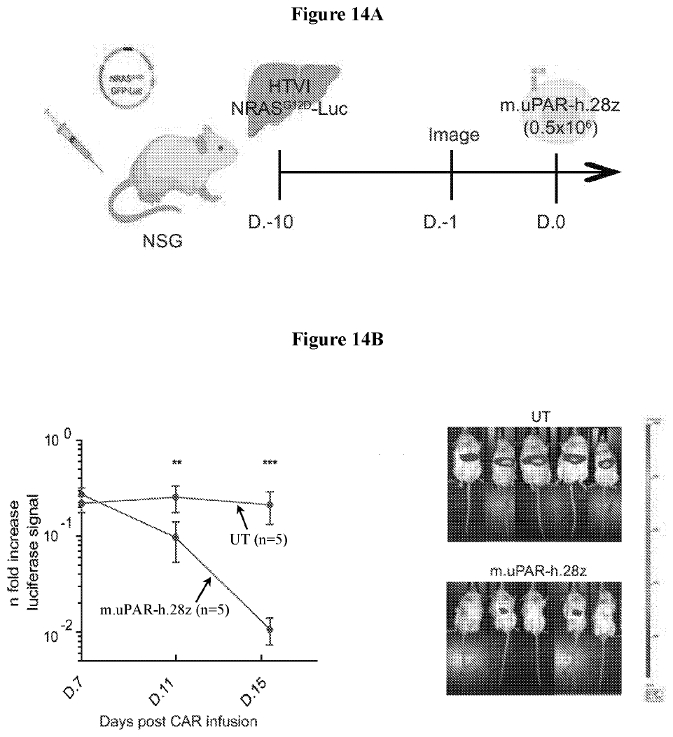

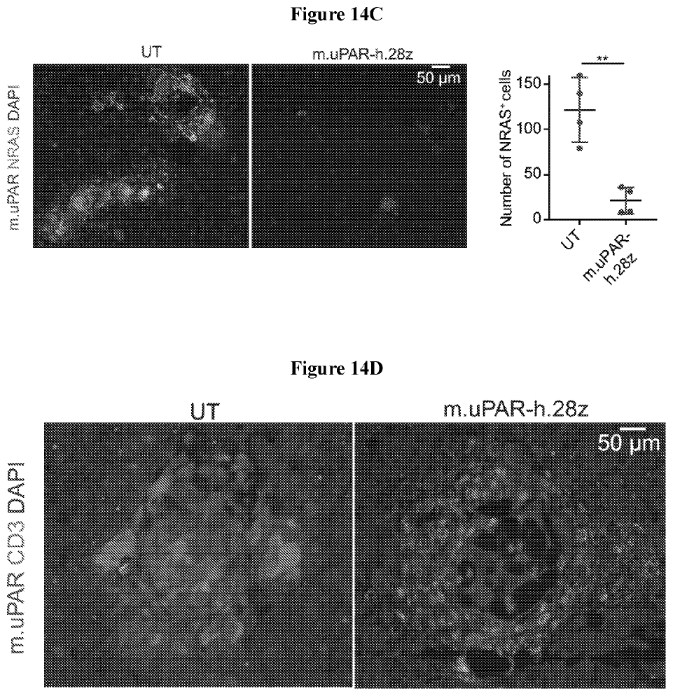

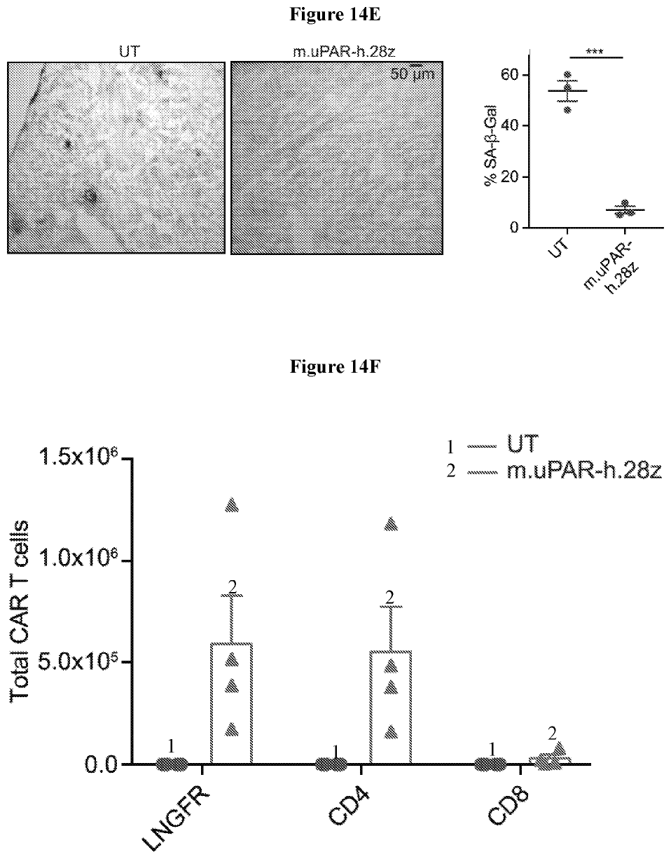

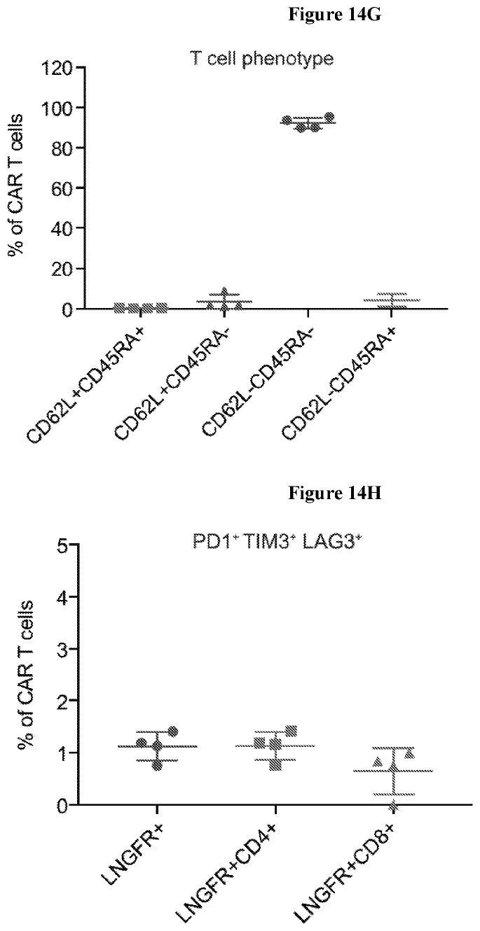

To study whether m.uPAR-28z CAR T cells could function as a bona fide senolytic in vivo, an experimental model of oncogene-induced senescence, in which somatic delivery of a transposon vector encoding Nras^{G12D}-Luciferase by HTVI successfully induces senescence in murine hepatocytes within 7-10 days after injection was employed. Kang et al., Nature 479: 547-551 (2011). While induction of a senescence-associated secretory phenotype (SASP) in immunocompetent mice facilitates the clearance of these senescent cells, they remain present in the liver of immunodeficient NOD-Scid-gamma (NSG) mice. Kang et al., Nature 479: 547-551 (2011). Successful HTVI-mediated transduction of murine hepatocytes in NSG mice was confirmed by bioluminescence imaging and followed by intravenous administration of 0.5×10⁶ m.uPAR-h.28z CAR⁺ T cells or untransduced T cells as controls (See FIG. 14A).

As shown in FIG. 14B, treatment with m.uPAR-h.28z CARs led to a profound decrease of bioluminescence signal within 10 days suggesting effective clearance of the senescent hepatocytes. This was independently confirmed by histological analyses shown in FIGS. 14C-14D, which demonstrated significantly decreased numbers of NRAS and uPAR co-expressing cells (P<0.01) as well as SA-β-gal positive cells (P<0.001) in the livers of NSG mice treated with m.uPAR-h.28z CAR T cells as compared to mice receiving untransduced T cells (FIGS. 14E-14F). Importantly, as shown in FIG. 14D, the infused T cells accumulated around the senescent hepatocytes within 7 days of their infusion. These liver-infiltrating CAR T cells comprised both CD4 and CD8 CAR T cells that displayed an effector memory phenotype (CD62L⁻CD45RA⁻) (FIG. 14G) with little evidence of T cell exhaustion (expression of PD1⁺ TIM3⁺LAG3⁺ on CAR⁺ T cells<2%, FIG. 14H) 15 days after their administration. Schietinger et al., Immunity 45: 389-401 (2016). Taken together, these results provide strong evidence that uPAR-28z CAR T cells efficiently access senescent hepatocytes and function as an effective senolytic agent in vivo.

Without wishing to be bound by theory, it is believed that as one of the main functions of the SASP is immunomodulation, it may affect the activity of the anti-uPAR CAR T cells. To determine this, the anti-mouse uPAR CAR T cells were co-cultured with supernatant from either proliferating or senescent murine ear fibroblasts for 24 hours (FIG. 15A). Interestingly, the CAR T cells cultured with the senescent supernatant demonstrated higher expression levels of activation markers (CD69, CD25, and LAG3) (FIG. 15B), which indicates that the SASP enhances CAR T cell activation. The results demonstrate that the engineered immune cells of the present technology are effective against senescence-associated pathologies.

Senescence contributes to a wide range of chronic tissue pathologies, including liver fibrosis as one of the most severe diseases and a direct precursor to cirrhosis and hepatocellular carcinoma (HCC). He and Sharpless, Cell 169: 1000-1011 (2017); Sharpless and Sherr, Nat Rev Cancer 15: 397-408 (2015); Lasry et al., Trends Immunol 36: 217-228 (2015). While senescence of hepatic stellate cells (HSCs) can facilitate fibrosis resolution if the damage-inducing stimulus is disrupted early during disease presentation, the accumulation of senescent cells over time exacerbates this pathology due to the chronic inflammatory effects of the SASP. Krizhanovsky et al., Cell 134: 657-667 (2008) Lujambio et al., Cell 153: 449-460 (2013). Consequently, genetic ablation of senescent cells promotes liver fibrosis resolution and leads to enhanced liver function. Krizhanovsky et al., Cell 134: 657-667 (2008); Lujambio et al., Cell 153: 449-460 (2013).

Accordingly, the CARs of the present technology are useful in the methods for treating or ameliorating the effects of a senescence-associated pathology in a subject in need thereof. Further, the methods disclosed herein are useful for detecting senescent cells in a biological sample obtained from a patient.

### Example 5: Therapeutic Efficacy of uPAR-CAR T Cells in Treating Liver Fibrosis and Lung Cancer In Vivo

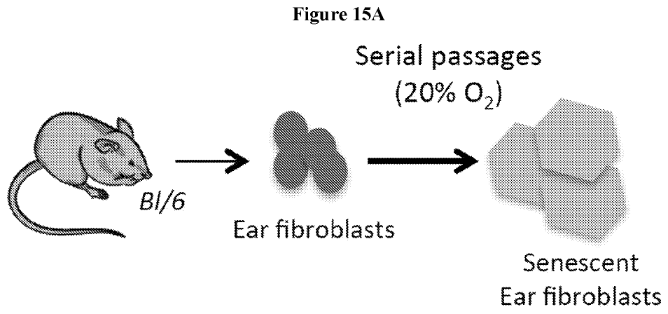

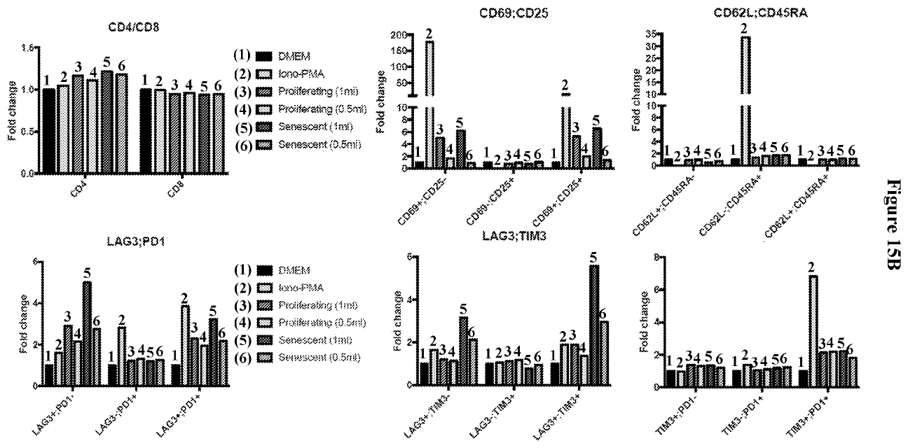

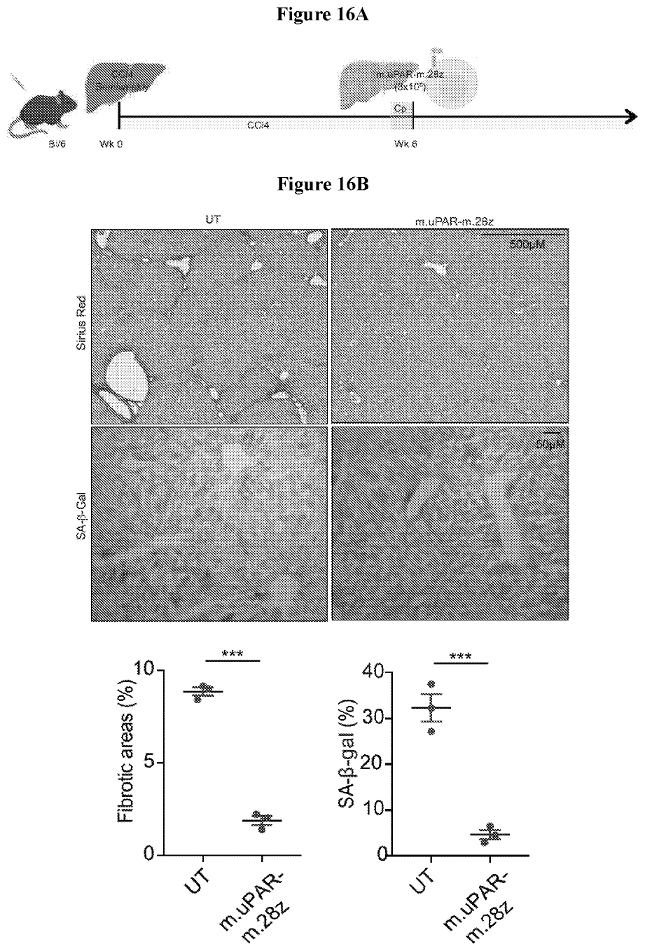

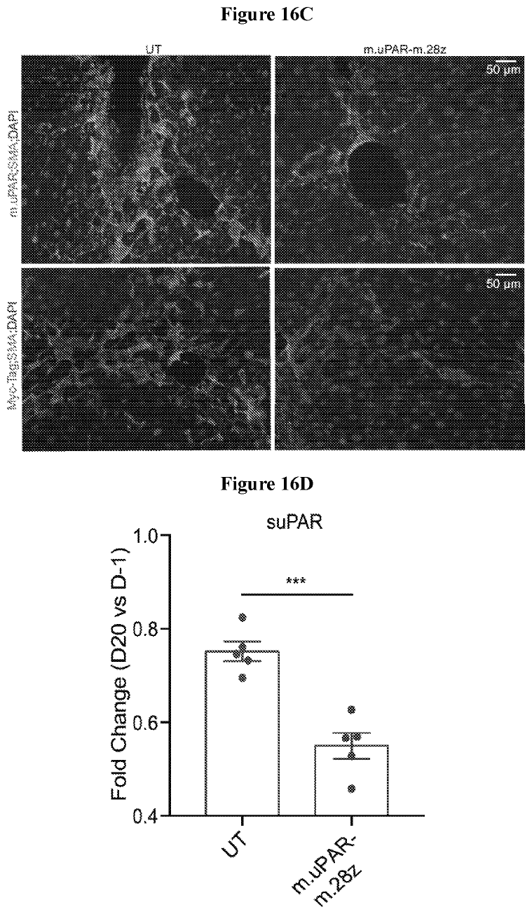

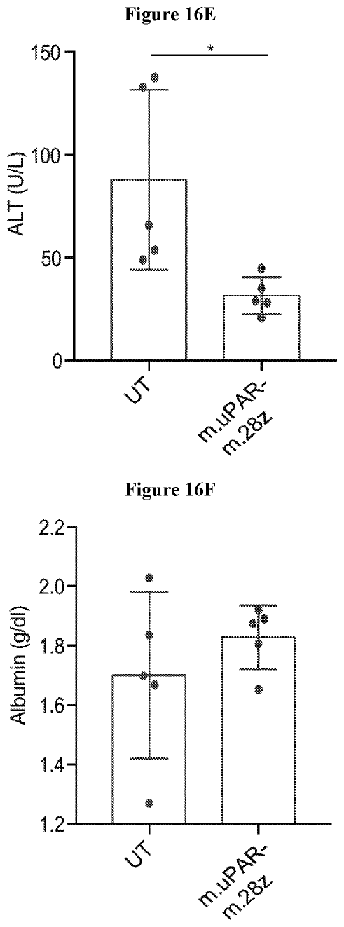

The therapeutic potential of m.uPAR-m.28z CAR T cells in a syngeneic and immunocompetent model of liver fibrosis was tested. To this end, a well-defined model of senescence induced by chronic CCl₄ exposure that produces notable fibrosis and decreased liver function within 6 weeks of treatment was used. Krizhanovsky et al., Cell 134: 657-667 (2008); Lujambio et al., Cell 153: 449-460 (2013). 3×10⁶ murine m.uPAR-m.28z CARs or untransduced T cells were then adoptively transferred into mice with established liver fibrosis after preconditioning with cyclophosphamide to increase T cell engraftment in the recipient mice (Krizhanovsky et al., Cell 134: 657-667 (2008); Lujambio et al., Cell 153: 449-460 (2013)). Liver function was monitored by serum alanine transaminase (ALT) and albumin levels, and the fibrosis was histologically assessed (FIG. 16A). CAR T cells did not show surface expression of mouse uPAR, indicating minimized risk of effector T cell fratricide and high potential for efficient and sustained CAR activity.

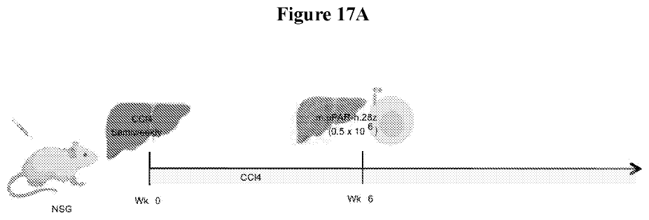

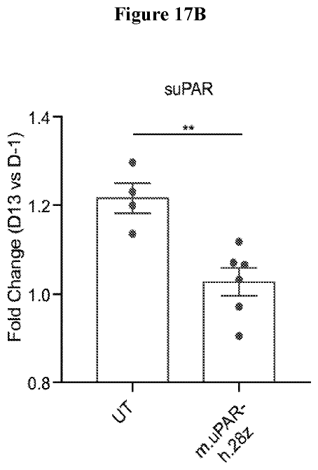

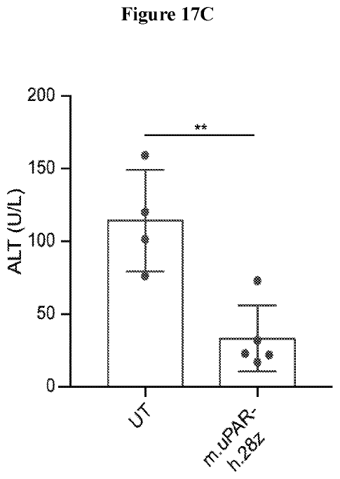

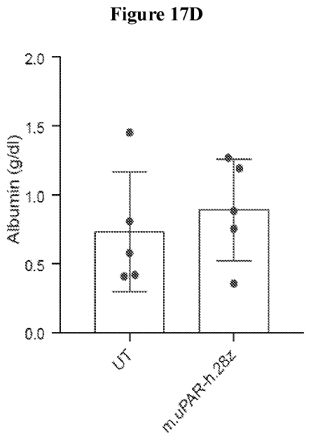

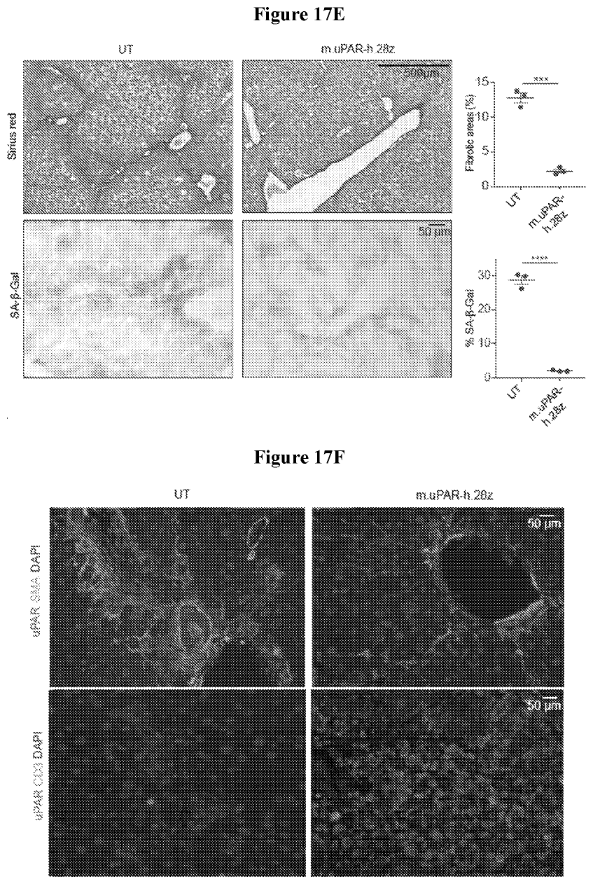

As shown in FIG. 16B, analysis of liver samples 20 days after T cell administration revealed a significant decrease of senescent cells in the livers of mice treated with m.uPAR-m.28z CAR T cells as compared to mice receiving untransduced T cells (P<0.001). These findings were corroborated by a considerable reduction of fibrosis as measured by Sirius red and smooth muscle actin positive areas (FIGS. 16B-16C). Of note, murine CAR T cells accumulated in the fibrotic liver areas of m.uPAR-m.28z-treated mice, as shown in FIG. 16C, which coincided with an improvement in liver function as shown by a significant decrease in serum ALT levels (FIGS. 16E-16F). Further corroborating these findings, as shown in FIGS. 17A-17F, infusion of human m.uPAR-h.28z CAR T cells in a comparable model of liver fibrosis in NSG mice also led to resolution of the fibrotic areas and recovery of liver function.

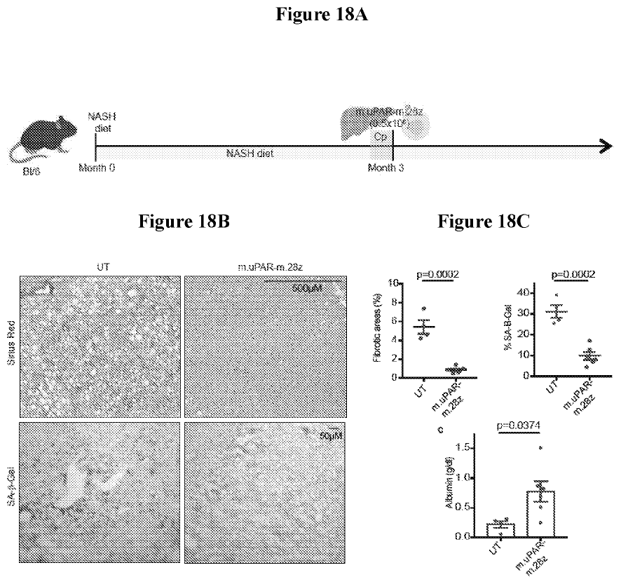

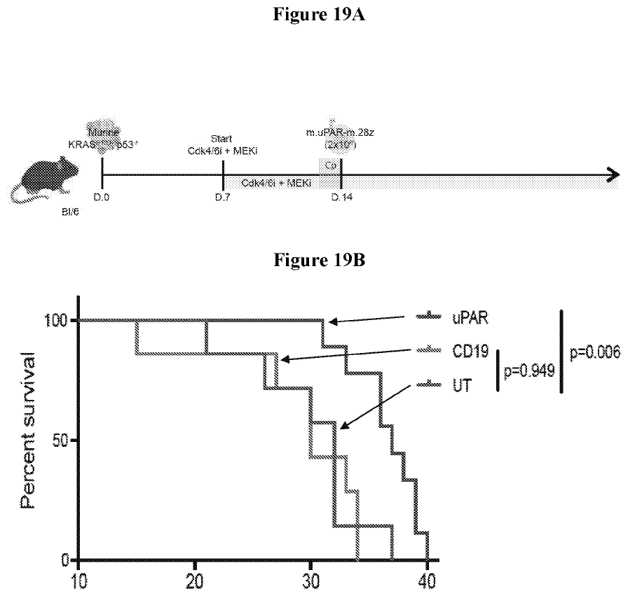

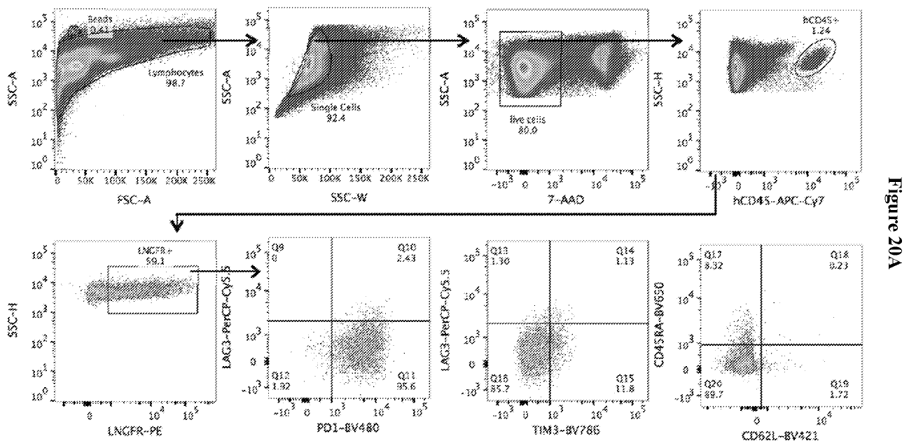

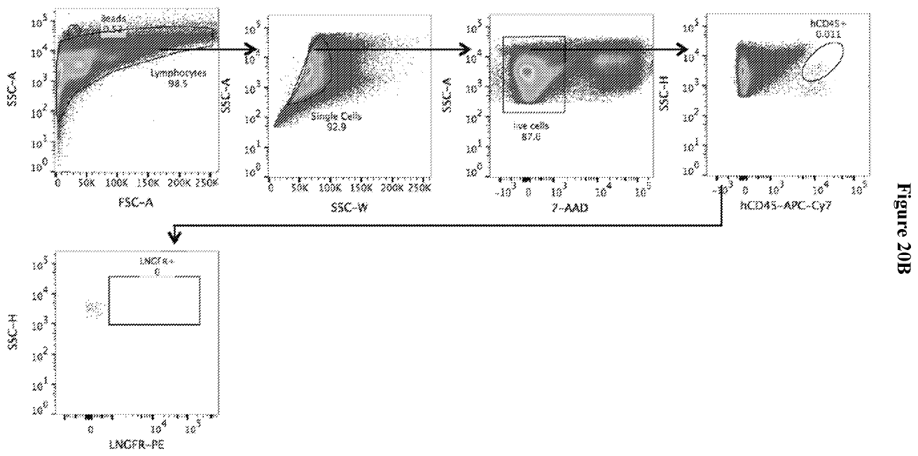

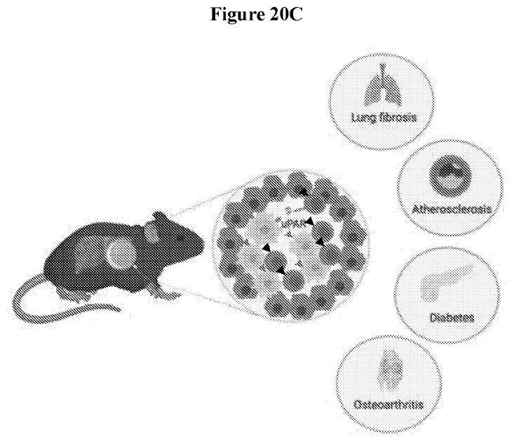

Consistent with the observed increase in suPAR levels in other senescence associated models (see above), as shown in FIGS. 5B and 16D, the appearance of liver fibrosis in CCl₄-treated mice was accompanied by an elevation of plasma suPAR levels relative to untreated controls. In contrast, treatment with m.uPAR-28z CAR T cells, but not untransduced T cells, resulted in a significant decrease in suPAR levels (FIGS. 16D and 17B), which correlated with the clearance of senescent cells and the resolution of liver fibrosis. Thus, suPAR levels in blood can serve as a biomarker of senolytic CAR T cell activity. As shown in FIGS. 18A-18C, senolytic CAR T cells also showed therapeutic efficacy in a model of liver fibrosis induced by Non-Alcoholic SteatoHepatitis (NASH). Moreover, the senolytic CAR T cells of the present technology allowed for a one-two punch senogenic-senolytic therapeutic approach in an in vivo model for lung cancer. See FIGS. 19A-19B.

Accordingly, the CARs of the present technology are useful in the methods for treating or ameliorating the effects of a senescence-associated pathology in a subject in need thereof.

### Example 6: In Vivo Senolytic Effects of uPAR-CAR T Cells Expressing uPA Fragments

T cells that express a chimeric antigen receptor that comprises an extracellular uPA fragment that is configured to bind to a uPAR polypeptide will be tested in several models of senescence. For example, NSG mice will receive a xenograft derived from a human tumor (e.g., lung or pancreatic cancer) and the tumor is subsequently induced to senesce (by radiotherapy, chemotherapy (e.g., doxorubicin) or targeted therapy (combined exposure to CDK4/6 and MEK inhibitors). See Examples 1-5 for detailed experimental methods. The mice will subsequently be treated with CAR T cells comprising the extracellular uPA fragment (e.g., SEQ ID NO: 59 or SEQ ID NO: 60). Likewise, the effects of these CAR T cells will be tested in humanized mouse models for liver fibrosis/cirrhosis. See Azuma et al., Nat Biotechnol. 25(8):903-10 (2007); Wilson et al., Stem Cell Res. 13(3 Pt A):404-12 (2014).

It is anticipated that mice receiving CAR T cells comprising the extracellular uPA fragment (e.g., SEQ ID NO: 59 or SEQ ID NO: 60) will show amelioration of one or more of the tested senescence-associated pathologies.

Accordingly, the CARs of the present technology are useful in the methods for treating or ameliorating the effects of a senescence-associated pathology in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. An engineered immune cell including a receptor that comprises
a) a uPAR antigen binding fragment comprising:
a V_{H}CDR1 sequence, a V_{H}CDR2 sequence, and a V_{H}CDR3 sequence of GFSLSTSGM (SEQ ID NO: 35), WWDDD (SEQ ID NO: 36), and IGGSSGYMDY (SEQ ID NO: 37), respectively; and
a V_{L}CDR1 sequence, a V_{L}CDR2 sequence, and a V_{L}CDR3 sequence of:
RASESVDSYGNSFMH (SEQ ID NO: 41), RASNLKS (SEQ ID NO: 42), and QQSNEDPWT (SEQ ID NO: 43) respectively; or
KASENVVTYVS (SEQ ID NO: 44), GASNRYT (SEQ ID NO: 45), and GQGYSYPYT (SEQ ID NO: 46), respectively, and/or
a nucleic acid encoding the receptor; or
(b) a uPAR antigen binding fragment comprising an amino acid sequence of SEQ ID NO: 52, and/or a nucleic acid encoding the receptor,
optionally wherein the engineered immune cell is derived from an autologous donor or an allogenic donor.

2. The engineered immune cell of claim 1, wherein the uPAR antigen binding fragment comprises a V_{H} amino acid sequence of SEQ ID NO: 48 and a V_{L} amino acid sequence of SEQ ID NO: 50 or SEQ ID NO: 51.

3. The engineered immune cell of claim 1, wherein the uPAR antigen binding fragment comprises an amino acid sequence selected from the group consisting of: SEQ ID NO: 53, and SEQ ID NO: 54; and/or a nucleic acid encoding the receptor.

4. The engineered immune cell of claim 1, wherein the receptor is a non-native cell receptor, a T cell receptor, a chimeric antigen receptor.

5.-6. (canceled)

7. The engineered immune cell of claim 1, wherein the nucleic acid encoding the receptor is operably linked to a promoter, optionally wherein the promoter is a constitutive promoter or a conditional promoter, preferably wherein the conditional promoter is induced by binding of the receptor to a uPAR antigen.

8.-14. (canceled)

15. The engineered immune cell of claim 4, wherein the chimeric antigen receptor comprises (i) an extracellular antigen binding domain; (ii) a transmembrane domain; and (iii) an intracellular domain.

16.-23. (canceled)

24. The engineered immune cell of claim 1, wherein the engineered immune cell is a lymphocyte, optionally wherein the lymphocyte is a T cell, a CD4+ T cell, a CD8+ T cell, a B cell, a tumor infiltrating lymphocyte, or a natural killer (NK) cell.

25.-59. (canceled)

60. A method for treating cancer in a subject in need thereof comprising administering to the subject an effective amount of the engineered immune cell of claim 1, wherein the subject is receiving or has received a senescence-inducing therapy, optionally wherein the cancer is selected from among breast cancer, endometrial cancer, ovarian cancer, colon cancer, lung cancer, stomach cancer, prostate cancer, renal cancer, pancreatic cancer, acute lymphoblastic leukemia (ALL), chronic lymphocytic leukemia (CLL), acute myeloid leukemia (AML), and metastases thereof.

61. (canceled)

62. A method for treating of inhibiting tumor growth or metastasis in a subject with cancer comprising contacting a tumor cell with an effective amount of the engineered immune cell of claim 1, optionally wherein the tumor is selected from among breast cancer, endometrial cancer, ovarian cancer, colon cancer, lung cancer, stomach cancer, prostate cancer, renal cancer, pancreatic cancer, acute lymphoblastic leukemia (ALL), chronic lymphocytic leukemia (CLL), acute myeloid leukemia (AML), and metastases thereof.

63.-69. (canceled)

70. A method for treating or ameliorating the effects of a senescence-associated pathology in a subject in need thereof comprising administering to the subject an effective amount of the engineered immune cell of claim 1, wherein the subject exhibits an increased accumulation of senescent cells compared to that observed in a healthy control subject, optionally wherein the senescence-associated pathology is lung fibrosis, atherosclerosis, Alzheimer's disease, diabetes, liver fibrosis, chronic kidney disease, aging, or osteoarthritis.

71. (canceled)

72. The method of claim 70, wherein the senescent cells exhibit a Senescence-Associated Secretory Phenotype (SASP), optionally wherein the Senescence-Associated Secretory Phenotype is induced by an oncogene or a drug.

73.-75. (canceled)

76. A method for detecting senescent cells in a biological sample obtained from a patient comprising:
detecting the presence of senescent cells in the biological sample by detecting uPAR and/or soluble uPAR (suPAR) polypeptide levels in the biological sample that are at least 5% higher compared to that observed in a reference sample, optionally wherein the reference sample is obtained from a healthy control subject or contains a predetermined level of the uPAR and/or suPAR polypeptide.

77. (canceled)

78. The method of claim 76, wherein the biological sample is mucus, saliva, bronchial alveolar lavage (BAL), bronchial wash (BW), whole blood, cerebrospinal fluid (CSF), urine, plasma, serum, lymph, semen, synovial fluid, tears, amniotic fluid, bile, aqueous humor, or a bodily fluid, and/or
wherein the uPAR and/or suPAR polypeptide levels are detected via Western Blotting, flow cytometry, Enzyme-linked immunosorbent assay (ELISA), immunoprecipitation, immunoelectrophoresis, immunostaining, isoelectric focusing, High-performance liquid chromatography (HPLC), or mass-spectrometry.

79.-92. (canceled)

93. A method for treating or ameliorating the effects of a senescence-associated pathology in a subject in need thereof comprising administering to the subject an effective amount of an engineered immune cell, wherein the engineered immune cell includes a receptor that comprises the amino acid of SEQ ID NO: 59 or SEQ ID NO: 60, and/or a nucleic acid encoding the receptor, wherein the subject exhibits an increased accumulation of senescent cells compared to that observed in a healthy control subject, optionally wherein the receptor is a non-native cell receptor, a T cell receptor or a chimeric antigen receptor.

94. The method of claim 93, wherein the senescence-associated pathology is lung fibrosis, atherosclerosis, Alzheimer's disease, diabetes, osteoarthritis, liver fibrosis, chronic kidney disease, breast cancer, endometrial cancer, colon cancer, lung cancer, stomach cancer, prostate cancer, renal cancer, pancreatic cancer, acute lymphoblastic leukemia (ALL), chronic lymphocytic leukemia (CLL), acute myeloid leukemia (AML), or metastases thereof.

95.-105. (canceled)

106. The method of claim 93, wherein the chimeric antigen receptor comprises (i) an extracellular uPA fragment that is configured to bind to a uPAR polypeptide; (ii) a transmembrane domain; and (iii) an intracellular domain, optionally wherein the extracellular uPA fragment comprises a human uPA fragment.

107.-113. (canceled)

114. The method of claim 93, wherein the engineered immune cell
is derived from an autologous donor or an allogenic donor; or
is a lymphocyte, optionally wherein the lymphocyte is a T cell, a CD4+ T cell, a CD8+ T cell, a B cell, a tumor infiltrating lymphocyte, or a natural killer (NK) cell.

115.-119. (canceled)

120. A method for treating cancer in a subject in need thereof comprising administering to the subject an effective amount of a senescence-inducing agent and an effective amount of an engineered immune cell, wherein the engineered immune cell
(i) is the engineered immune cell of claim 1: or
(ii) includes a receptor that comprises the amino acid of SEQ ID NO: 59 or SEQ ID NO: 60, and/or a nucleic acid encoding the receptor.

121. The method of claim 120, wherein the senescence-inducing agent is doxorubicin, ionizing radiation therapy, combination therapy with a MEK inhibitor and a CDK4/6 inhibitor, or combination therapy with a CDCl₇ inhibitor and a mTOR inhibitor, optionally wherein
the MEK inhibitor is selected from the group consisting of PD-325901, TAK-733, CI-1040 (PD184352), PD0325901, MEK162, AZD8330, GDC-0623, refametinib, pimasertib, RO4987655, RO5126766, WX-554, HL-085, CInQ-03, G-573, PD184161, PD318088, PD98059, RO5068760, U0126, and SL327; or
the CDK4/6 inhibitor is selected from the group consisting of palbociclib, ribociclib, and abemaciclib; or
the CDCl₇ inhibitor is selected from the group consisting of TAK-931, PHA-767491, XL413, 1H-pyrrolo[2,3-b]pyridines, 2,3-dihydrothieno[3,2-d]pyrimidin-4(1H)-ones, furanone derivatives, and trisubstituted thiazoles, pyrrolopyridinones.

122.-125. (canceled)
